(19) United States
(12) Patent Application Publication
(10) Pub. No.: US 2025/0351831 A1
(43) Pub. Date: Nov. 20, 2025

(54) ENDOPHYTE COMPOSITIONS AND METHODS FOR IMPROVED PLANT HEALTH

(71) Applicant: INDIGO AG, INC., Charlestown, MA (US)

(72) Inventors: Shib Sankar BASU, Apex, NC (US); David A. HUBERT, Durham, NC (US); Sara B. COLLINS, Swampscott, MA (US); Brandon M. SATINSKY, Brookline, MA (US)

(21) Appl. No.: 18/717,145

(22) PCT Filed: Dec. 7, 2022
(86) PCT No.: PCT/US2022/081127

§ 371 Date: Jun. 6, 2024

Related U.S. Application Data
(60) Provisional application No. 63/265,091, filed on Dec. 7, 2021.

(51) Int. Cl. A01N 63/20; A01P 21/00; C12N 1/20; C12R 1/01

(52) U.S. Cl. CPC A01N 63/20; A01P 21/00; C12N 1/20; C12R 2001/01

(57) ABSTRACT

This invention relates to compositions and methods for improving plant health, including treatment formulations plant comprising one or more endophytes.

### CROSS REFERENCE TO RELATED APPLICATIONS

This application is a U.S. National Phase Application under 35 U.S.C. § 371 of International Application No. PCT/US2022/081127 filed Dec. 7, 2022, which claims the benefit of U.S. Provisional Application No. 63/265,091, filed Dec. 7, 2021, which are hereby incorporated by reference in their entirety.

### SEQUENCE LISTING

The instant application contains a Sequence Listing which has been submitted via the USPTO Patent Center and is hereby incorporated by reference in its entirety. Said XML copy, created on Dec. 23, 2024, is named 10242PCTWO1_Sequence_Listing_Updated_2024-12-23.xml, and is 3,175,846 bytes in size.

### BACKGROUND

According to the United Nations Food and Agriculture Organization, the world's population will exceed 9.6 billion people by the year 2050, which will require significant improvements in agriculture to meet growing food demands. There is a need for improved methods and agricultural plants that will enable a near doubling of food production with fewer resources and more environmentally sustainable inputs, and for plants with improved responses to various stresses.

### SUMMARY OF INVENTION

Disclosed herein are methods of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one tailocin gene cluster. In some embodiments, the tailocin gene cluster comprises one or more polynucleotide sequences at least 97% identical to one or more of SEQ ID NOs. 287-933. In some embodiments, the tailocin gene cluster comprises one or more polynucleotide sequences at least 97% identical to one or more of SEQ ID NOs. 287-289. In some embodiments, the tailocin gene cluster comprises one or more polynucleotide sequences at least 97% identical to one or more of SEQ ID NOs. 290-343. In some embodiments, the tailocin gene cluster comprises one or more polynucleotide sequences at least 97% identical to one or more of SEQ ID NOs. 375-452. In some embodiments, the one or more endophytes comprise a plasmid comprising one or more polynucleotide sequences at least 97% identical to one or more of SEQ ID NOs. 287-933. Optionally, the one or more endophytes are capable of producing one or more proteins whose amino acid sequences are selected from SEQ ID NOs. 1318-1922. Optionally, the one or more endophytes are capable of producing one or more proteins whose amino acid sequences are selected from SEQ ID NOs. 1318-1321. Optionally, the one or more endophytes are capable of producing one or more proteins whose amino acid sequences are selected from SEQ ID NOs. 1322-1342. In some embodiments, the one or more endophytes comprise a plasmid comprising an open reading frame encoding a protein whose amino acid sequence is selected from SEQ ID 1318-1922. In some embodiments, the one or more endophytes comprise one or more polynucleotide sequences at least 97% identical to one or more of SEQ ID NOs. 287-933, and the one or more endophytes are capable of producing one or more proteins whose amino acid sequences are selected from SEQ ID NOs. 1318-1922. In some embodiments the trait of agronomic importance is biotic stress tolerance, for example biotic stress tolerance is increased early emergence, increased emergence, increased plant height, increased root weight, and or increased shoot weight, in a growth environment comprising one or more pests or pathogens. In some embodiments, the pathogen is of the genus Dreschlera, Bipolaris, Pythium, Rhizoctonia, and or Fusarium. In some embodiments, the plant element is a seed of a cereal plant. In some embodiments, the plant element is a seed of a corn plant. In some embodiments, the plant element is a seed of a winter wheat plant. In some embodiments, the plant element is a seed of a soybean plant.

Disclosed herein are methods of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one gene of a Type VI secretion system and the gene polynucleotide sequence is at least 97% identical to one or more of SEQ ID NOs. 934-1014. In some embodiments, the Type VI secretion system comprises one or more polynucleotide sequences at least 97% identical to one or more of SEQ ID NOs. 934-941. In some embodiments, the Type VI secretion system comprises one or more polynucleotide sequences at least 97% identical to one or more of SEQ ID NOs. 942-962. In some embodiments, the one or more endophytes comprise a plasmid comprising one or more polynucleotide sequences at least 97% identical to one or more of SEQ ID NOs. 934-1014. Optionally, the one or more endophytes are capable of producing one or more proteins whose amino acid sequences are selected from SEQ ID NOs. 1923-1981. Optionally, the one or more endophytes are capable of producing one or more proteins whose amino acid sequences are selected from SEQ ID NOs. 1923-1930. Optionally, the one or more endophytes are capable of producing one or more proteins whose amino acid sequences are selected from SEQ ID NOs. 1931-1947. In some embodiments, the one or more endophytes comprise at least one gene of a Type VI secretion system and the gene polynucleotide sequence is at least 97% identical to one or more of SEQ ID NOs. 934-1014, and the one or more endophytes are capable of producing one or more proteins whose amino acid sequences are selected from SEQ ID NOs. 1923-1981. In some embodiments, the one or more endophytes comprise a plasmid comprising an open reading frame encoding a protein whose amino acid sequence is selected from SEQ IDs. 1923-1981. In some embodiments the trait of agronomic importance is biotic stress tolerance, for example biotic stress tolerance is increased early emergence, increased emergence, increased plant height, increased root weight, and or increased shoot weight, in a growth environment comprising one or more pests or pathogens. In some embodiments, the pathogen is of the genus Dreschlera, Bipolaris, Pythium, Rhizoctonia, and or Fusarium. In some embodiments, the plant element is a seed of a cereal plant. In some embodiments, the plant element is a seed of a corn plant. In some embodiments, the plant element is a seed of a winter wheat plant. In some embodiments, the plant element is a seed of a soybean plant.

Disclosed herein are methods of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one Type VI secretion system putative effector gene and the gene polynucleotide sequence is at least 97% identical to one or more of SEQ ID NOs. 1015-1089. In some embodiments, the Type VI secretion system putative effector comprises one or more polynucleotide sequences at least 97% identical to one or more of SEQ ID NOs. 1015-1022. In some embodiments, the one or more endophytes comprise a plasmid comprising one or more polynucleotide sequences at least 97% identical to one or more of SEQ ID NOs. 1015-1089. Optionally, the one or more endophytes are capable of producing one or more proteins whose amino acid sequences are selected from SEQ ID NOs. 1982-2051. Optionally, the one or more endophytes are capable of producing one or more proteins whose amino acid sequences are selected from SEQ ID NOs. 1982-1989. In some embodiments, the one or more endophytes comprise at least one Type VI secretion system putative effector gene and the gene polynucleotide sequence is at least 97% identical to one or more of SEQ ID NOs. 1015-1089, and the one or more endophytes are capable of producing one or more proteins whose amino acid sequences are selected from SEQ ID NOs. 1982-2051. In some embodiments, the one or more endophytes comprise a plasmid comprising an open reading frame encoding a protein whose amino acid sequence is selected from SEQ IDs. 1982-2051. In some embodiments the trait of agronomic importance is biotic stress tolerance, for example biotic stress tolerance is increased early emergence, increased emergence, increased plant height, increased root weight, and or increased shoot weight, in a growth environment comprising one or more pests or pathogens. In some embodiments, the pathogen is of the genus Dreschlera, Bipolaris, Pythium, Rhizoctonia, and or Fusarium. In some embodiments, the plant element is a seed of a cereal plant. In some embodiments, the plant element is a seed of a corn plant. In some embodiments, the plant element is a seed of a winter wheat plant. In some embodiments, the plant element is a seed of a soybean plant.

Disclosed herein are methods of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one flagellin gene at least 97% identical to one or more of SEQ ID NOs. 1-239. In some embodiments, the endophyte comprises one or more polynucleotide sequences at least 97% identical to one or more of SEQ IDs. 1-23. In some embodiments, the endophyte comprises one or more polynucleotide sequences at least 97% identical to one or more of SEQ IDs. 14-28. In some embodiments, the one or more endophytes comprise a plasmid comprising a polynucleotide sequence at least 97% identical to one or more of SEQ ID NOs. 1-239. Optionally, the one or more endophytes are capable of producing one or more proteins whose amino acid sequences are selected from SEQ ID NOs. 1090-1272. Optionally, the one or more endophytes are capable of producing one or more proteins whose amino acid sequences are selected from SEQ ID NOs. 1090-1106. Optionally, the one or more endophytes are capable of producing one or more proteins whose amino acid sequences are selected from SEQ ID NOs. 1107-1114. In some embodiments, the one or more endophytes comprise at least one flagellin gene at least 97% identical to one or more of SEQ ID NOs. 1-239, and the one or more endophytes are capable of producing one or more proteins whose amino acid sequences are selected from SEQ ID NOs. 1090-1272. In some embodiments, the one or more endophytes comprise a plasmid comprising an open reading frame encoding a protein whose amino acid sequence is selected from SEQ IDs. 1090-1272. In some embodiments the trait of agronomic importance is biotic stress tolerance, for example biotic stress tolerance is increased early emergence, increased emergence, increased plant height, increased root weight, and or increased shoot weight, in a growth environment comprising one or more pests or pathogens. In some embodiments, the pathogen is Dreschlera, Bipolaris, Pythium, Rhizoctonia, and or Fusarium. In some embodiments, the plant element is a seed of a cereal plant. In some embodiments, the plant element is a seed of a corn plant. In some embodiments, the plant element is a seed of a winter wheat plant. In some embodiments, the plant element is a seed of a soybean plant.

Disclosed herein are methods of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one O-antigen biosynthesis gene at least 97% identical to one or more of SEQ ID NOs. 240-253. In some embodiments, the one or more endophytes comprise at least one O-antigen biosynthesis gene at least 97% identical to one or more of SEQ ID NOs. 240-243. Optionally, the one or more endophytes are capable of producing one or more proteins whose amino acid sequences are selected from SEQ ID NOs. 1273-1285. In some embodiments, the one or more endophytes comprise at least one O-antigen biosynthesis gene at least 97% identical to one or more of SEQ ID NOs. 240-253, and the one or more endophytes are capable of producing one or more proteins whose amino acid sequences are selected from SEQ ID NOs. 1273-1285. In some embodiments the trait of agronomic importance is biotic stress tolerance, for example biotic stress tolerance is increased early emergence, increased emergence, increased plant height, increased root weight, and or increased shoot weight, in a growth environment comprising one or more pests or pathogens. In some embodiments, the pathogen is Dreschlera, Bipolaris, Pythium, Rhizoctonia, and or Fusarium. In some embodiments, the plant element is a seed of a cereal plant. In some embodiments, the plant element is a seed of a corn plant. In some embodiments, the plant element is a seed of a winter wheat plant. In some embodiments, the plant element is a seed of a soybean plant.

Disclosed herein are methods of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one pseudaminic acid biosynthesis gene at least 97% identical to one or more of SEQ ID NOs. 254-286. In some embodiments, the one or more endophytes comprise at least one pseudaminic acid biosynthesis gene at least 97% identical to one or more of SEQ ID NOs. 254-262. Optionally, the one or more endophytes are capable of producing one or more proteins whose amino acid sequences are selected from SEQ ID NOs. 1286-1317. In some embodiments, the one or more endophytes comprise at least one pseudaminic acid biosynthesis gene at least 97% identical to one or more of SEQ ID NOs. 254-286, and the one or more endophytes are capable of producing one or more proteins whose amino acid sequences are selected from SEQ ID NOs. 1286-1317. In some embodiments the trait of agronomic importance is biotic stress tolerance, for example biotic stress tolerance is increased early emergence, increased emergence, increased plant height, increased root weight, and or increased shoot weight, in a growth environment comprising one or more pests or pathogens. In some embodiments, the pathogen is Dreschlera, Bipolaris, Pythium, Rhizoctonia, and or Fusarium. In some embodiments, the plant element is a seed of a cereal plant. In some embodiments, the plant element is a seed of a corn plant. In some embodiments, the plant element is a seed of a winter wheat plant. In some embodiments, the plant element is a seed of a soybean plant.

In some embodiments, the method additionally comprises the step of placing the plant element in or on a growth medium. In some embodiments, the one or more endophytes are heterologously disposed to a plant element prior to placing the treated plant element in or on a growth medium. In some embodiments, the one or more endophytes are heterologously disposed to a plant element after placing the plant elements in or on a growth medium. In some embodiments, the one or more endophytes are heterologously disposed to a plant element concurrently with placing the plant elements in or on a growth medium.

In some embodiments, the one or more endophytes are heterologously disposed to a plant element at least two times. In some embodiments, the one or more endophytes are heterologously disposed to a plant element via a seed treatment or soil pre-treatment and one or more foliar applications. In some embodiments, the one or more endophytes are heterologously disposed to a plant element via a seed treatment or soil pre-treatment and one or more floral applications. In some embodiments, the one or more endophytes are heterologously disposed to a plant element via one or more seed treatments or soil pre-treatments, one or more foliar applications, and one or more floral applications. In some embodiments, the one or more endophytes are heterologously disposed to a plant element via seed treatment, root wash, seedling soak, foliar application, floral application, soil inoculum, in-furrow application, sidedress application, soil pre-treatment, wound inoculation, drip tape irrigation, vector-mediation inoculation, injection, osmopriming, hydroponics, aquaponics, aeroponics, or combinations thereof.

In some embodiments, the one or more endophytes are heterologously disposed to a plant element of a different plant variety from the variety of the plant element from which the one or more endophytes were obtained. In some embodiments, the one or more endophytes are heterologously disposed to a plant element of the same plant variety as the variety of the plant element from which the one or more endophytes were obtained. In some embodiments, the one or more endophytes are heterologously disposed to a plant element of a different plant species from the species of the plant element from which the one or more endophytes were obtained. In some embodiments, the one or more endophytes are heterologously disposed to a plant element of the same plant species as the species of the plant element from which the one or more endophytes were obtained.

In some embodiments, the plant elements are allowed to germinate. In some embodiments, the plant elements are grown to yield.

In another aspect, disclosed herein are synthetic compositions, comprising one or more endophytes heterologously disposed to a treatment formulation, wherein the one or more endophytes comprise at comprise at least one tailocin gene cluster.

In another aspect, disclosed herein are synthetic compositions, comprising one or more endophytes heterologously disposed to a treatment formulation, wherein the one or more endophytes comprise at least one polynucleotide sequence that is at least 97% identical to one or more of SEQ ID NOs. 1-1089. Optionally, the one or more endophytes are capable of producing one or more proteins whose amino acid sequences are selected from SEQ ID NOs. 1090-2051. In some embodiments, the composition additionally comprises a plant element. In some embodiments, the one or more endophytes are capable of improving a trait of agronomic importance in a plant derived from the plant element (for example, when grown from a treated seed) relative to a plant derived from a reference plant element. In some embodiments, the plant element is a dicot. In some embodiments, the plant element is a monocot.

In some embodiments synthetic compositions, comprise one or more endophytes heterologously disposed to a treatment formulation, wherein the one or more endophytes comprise at least one gene of a tailocin gene cluster and the tailocin gene polynucleotide sequence is at least 97% identical to one or more of SEQ ID NOs. 287-933. In some embodiments, the one or more endophytes comprise at least one gene of a tailocin gene cluster and the tailocin gene polynucleotide sequence is at least 97% identical to one or more of SEQ ID NOs. 287-289. In some embodiments, the one or more endophytes comprise at least one gene of a tailocin gene cluster and the tailocin gene polynucleotide sequence is at least 97% identical to one or more of SEQ ID NOs. 290-343. In some embodiments, the one or more endophytes comprise at least one gene of a tailocin gene cluster and the tailocin gene polynucleotide sequence is at least 97% identical to one or more of SEQ ID NOs. 344-374. In some embodiments, the one or more endophytes comprise at least one gene of a tailocin gene cluster and the tailocin gene polynucleotide sequence is at least 97% identical to one or more of SEQ ID NOs. 375-452. In some embodiments the one or more endophytes are genetically modified to comprise a polynucleotide sequence selected from the group consisting of SEQ ID NOs. 287-933. In some embodiments the one or more endophytes are genetically modified to comprise a polynucleotide sequence selected from the group consisting of SEQ ID NOs. 287-289. In some embodiments the one or more endophytes are genetically modified to comprise a polynucleotide sequence selected from the group consisting of SEQ ID NOs. 290-343. In some embodiments the one or more endophytes are genetically modified to comprise a polynucleotide sequence selected from the group consisting of SEQ ID Nos. 344-374. In some embodiments the one or more endophytes are genetically modified to comprise a polynucleotide sequence selected from the group consisting of SEQ ID NOs. 375-452. In some embodiments the one or more endophytes is genetically modified to produce one or more proteins whose amino acid sequences are selected from SEQ ID NOs. 1318-1922. In some embodiments the one or more endophytes is genetically modified to produce one or more proteins whose amino acid sequences are selected from SEQ ID NOs. 1318-1321. In some embodiments the one or more endophytes is genetically modified to produce one or more proteins whose amino acid sequences are selected from SEQ ID NOs. 1322-1342. In some embodiments, the synthetic composition further comprises a plant element. In some embodiments, the plant element is a seed of a dicot plant. In some embodiments, the plant element is a seed of a monocot plant.

In some embodiments synthetic compositions, comprise one or more endophytes heterologously disposed to a treatment formulation, wherein the one or more endophytes comprise at least one gene of a Type VI secretion system and the gene polynucleotide sequence is at least 97% identical to one or more of SEQ ID NOs. 934-1014. In some embodiments, the one or more endophytes are capable of producing one or more proteins whose amino acid sequences are selected from SEQ ID NOs. 1923-1981. In some embodiments the one or more endophytes are genetically modified to comprise a polynucleotide sequence selected from the group consisting of SEQ ID NOs. 934-1014. In some embodiments the one or more endophytes is genetically modified to produce one or more proteins whose amino acid sequences are selected from SEQ ID NOs. 1923-1981. In some embodiments, the synthetic composition further comprises a plant element. In some embodiments, the plant element is a seed of a dicot plant. In some embodiments, the plant element is a seed of a monocot plant.

In some embodiments synthetic compositions, comprise one or more endophytes heterologously disposed to a treatment formulation, wherein the one or more endophytes comprise at least one Type VI secretion system putative effector gene and the gene polynucleotide sequence is at least 97% identical to one or more of SEQ ID NOs. 1015-1089. In some embodiments, the one or more endophytes are capable of producing one or more proteins whose amino acid sequences are selected from SEQ ID NOs. 1982-2051. In some embodiments the one or more endophytes are genetically modified to comprise a polynucleotide sequence selected from the group consisting of SEQ ID NOs. 1015-1089. In some embodiments the one or more endophytes is genetically modified to produce one or more proteins whose amino acid sequences are selected from SEQ ID NOs. 1982-2051. In some embodiments, the synthetic composition further comprises a plant element. In some embodiments, the plant element is a seed of a dicot plant. In some embodiments, the plant element is a seed of a monocot plant.

In some embodiments synthetic compositions, comprise one or more endophytes heterologously disposed to a treatment formulation, wherein the one or more endophytes comprise at least one flagellin gene at least 97% identical to one or more of SEQ ID NOs. 1-239. In some embodiments, the one or more endophytes are capable of producing one or more proteins whose amino acid sequences are selected from SEQ ID NOs. 1090-1272. In some embodiments the one or more endophytes are genetically modified to comprise a polynucleotide sequence selected from the group consisting of SEQ ID NOs. 1-239. In some embodiments the one or more endophytes is genetically modified to produce one or more proteins whose amino acid sequences are selected from SEQ ID NOs. 1090-1272 and combinations thereof. In some embodiments, the synthetic composition further comprises a plant element. In some embodiments, the plant element is a seed of a dicot plant. In some embodiments, the plant element is a seed of a monocot plant.

In some embodiments synthetic compositions, comprise one or more endophytes heterologously disposed to a treatment formulation, wherein the one or more endophytes comprise at least one O-antigen biosynthesis gene at least 97% identical to one or more of SEQ ID NOs. 240-253. In some embodiments, the one or more endophytes are capable of producing one or more proteins whose amino acid sequences are selected from SEQ ID NOs. 1273-1285. In some embodiments the one or more endophytes are genetically modified to comprise a polynucleotide sequence selected from the group consisting of SEQ ID NOs. 240-253. In some embodiments the one or more endophytes is genetically modified to produce one or more proteins whose amino acid sequences are selected from SEQ ID NOs. 1273-1285. In some embodiments, the synthetic composition further comprises a plant element. In some embodiments, the plant element is a seed of a dicot plant. In some embodiments, the plant element is a seed of a monocot plant.

In some embodiments synthetic compositions, comprise one or more endophytes heterologously disposed to a treatment formulation, wherein the one or more endophytes comprise at least one pseudaminic acid biosynthesis gene at least 97% identical to one or more of SEQ ID NOs. 254-286. In some embodiments, the one or more endophytes are capable of producing one or more proteins whose amino acid sequences are selected from SEQ ID NOs. 1286-1317. In some embodiments the one or more endophytes are genetically modified to comprise a polynucleotide sequence selected from the group consisting of SEQ ID NOs. 254-286. In some embodiments the one or more endophytes is genetically modified to produce one or more proteins whose amino acid sequences are selected from SEQ ID NOs. 1286-1317. In some embodiments, the synthetic composition further comprises a plant element. In some embodiments, the plant element is a seed of a dicot plant. In some embodiments, the plant element is a seed of a monocot plant.

In some embodiments, a synthetic composition comprises one or more endophytes heterologously disposed to a treatment formulation, wherein the endophytes comprise one or more of:
a flagellin gene having at least 97% identity to one or more of SEQ IDs. 1-8 or 14-16, an O-antigen biosynthesis gene having at least 97% identity to one or more of SEQ IDs. 240-243,
a pseudoaminic acid biosynthesis gene having at least 97% identity to one or more of SEQ IDs. 256-262,
a gene of a tailocin gene cluster having at least 97% identity to one or more of SEQ IDs. 288-342,
a gene of a Type IV secretion system having at least 97% identity to one or more of SEQ IDs. 938-941, and
a gene of a Type IV secretion system putative effector having at least 97% identity to one or more of SEQ IDs. 1018-1022,
wherein the endophyte in the synthetic combination is capable of improving a trait of agronomic importance in a plant or plant element heterologously disposed to the synthetic composition.

In some embodiments, a synthetic composition comprises one or more endophytes heterologously disposed to a treatment formulation, wherein the endophytes comprise one or more of:
a flagellin gene having at least 97% identity to one or more of SEQ IDs. 9, 55, 56, 130-133,
a pseudoaminic acid biosynthesis gene having at least 97% identity to one or more of SEQ IDs. 254-255,
a gene of a tailocin gene cluster having at least 97% identity to one or more of SEQ IDs. 288-289 or 566-605,
a gene of a Type IV secretion system having at least 97% identity to one or more of SEQ IDs. 974-977, and
a gene of a Type IV secretion system putative effector having at least 97% identity to one or more of SEQ IDs. 1054-1057,
wherein the endophyte in the synthetic combination is capable of improving a trait of agronomic importance in a plant or plant element heterologously disposed to the synthetic composition.

In some embodiments, a synthetic composition comprises one or more endophytes heterologously disposed to a treatment formulation, wherein the endophytes comprise one or more of:
a flagellin gene having at least 97% identity to one or more of SEQ IDs. 9, 11-13, 17-18, 20-23,
a pseudoaminic acid biosynthesis gene having at least 97% identity to one or more of SEQ IDs. 254-255,
a gene of a tailocin gene cluster having at least 97% identity to one or more of SEQ IDs. 287, 343, 382-390,
a gene of a Type IV secretion system having at least 97% identity to one or more of SEQ IDs. 934-937, and
a gene of a Type IV secretion system putative effector having at least 97% identity to one or more of SEQ IDs. 1015-1017 or 1024,
wherein the endophyte in the synthetic combination is capable of improving a trait of agronomic importance in a plant or plant element heterologously disposed to the synthetic composition.

In some embodiments, a synthetic composition comprises one or more endophytes heterologously disposed to a treatment formulation, wherein the endophytes comprise one or more of:
a flagellin gene having at least 97% identity to one or more of SEQ IDs. 10 or 19, a gene of a tailocin gene cluster having at least 97% identity to one or more of SEQ IDs. 375-381, and
a gene of a Type IV secretion system having at least 97% identity to SEQ ID. 944, wherein the endophyte in the synthetic combination is capable of improving a trait of agronomic importance in a plant or plant element heterologously disposed to the synthetic composition.

In some embodiments, a synthetic composition comprises one or more endophytes heterologously disposed to a treatment formulation, wherein the endophytes comprise one or more polynucleotide sequences at least 97% identical to one or more of SEQ ID NOs. 1-8, and the one or more endophytes are capable of producing one or more proteins whose amino acid sequences are selected from SEQ ID NOs. 1090-1096, wherein the endophyte in the synthetic combination is capable of improving biotic stress tolerance in an environment containing pathogenic Fusarium in a plant or plant element heterologously disposed to the synthetic composition.

In some embodiments, a synthetic composition comprises one or more endophytes heterologously disposed to a treatment formulation, wherein the endophytes comprise one or more polynucleotide sequences at least 97% identical to one or more of SEQ ID NOs. 9-13, 254, 255, 287, 934-937, 1015-1017, 1-8, 24-28, 75-85, 365-374, 442-452, 959-962, 1023, 1038-1041, and the one or more endophytes are capable of producing one or more proteins whose amino acid sequences are selected from SEQ ID NOs. 1097-1103, 1286, 1287, 1318, 1319, 1923-1926, 1982-1984, 1090-1096, 1113, 1114, 1149-1157, 1402-1409, 1471-1483, 1946-1947, 1990, 2005, 2006, wherein the endophyte in the synthetic combination is capable of improving biotic stress tolerance in an environment containing pathogenic Rhizocotina in a plant or plant element heterologously disposed to the synthetic composition.

In some embodiments, a synthetic composition comprises one or more endophytes heterologously disposed to a treatment formulation, wherein the endophytes comprise one or more polynucleotide sequences at least 97% identical to one or more of SEQ ID NOs. 1-18, 240-243, 254-262, 287-364, 934-943, 1015-1022, 19-74, 365-374, 391-441, 945-958, 1023, 1025-1037, and the one or more endophytes are capable of producing one or more proteins whose amino acid sequences are selected from SEQ ID NOs. 1090-1106, 1112, 1273-1276, 1286-1294, 1318-1401, 1923-1934, 1982-1989, 1107-1111, 1113-1148, 1402-1409, 1426-1470, 1936-1945, 1990, 1992-2004, wherein the endophyte in the synthetic combination is capable of improving biotic stress tolerance in an environment containing pathogenic Pythium in a plant or plant element heterologously disposed to the synthetic composition.

In some embodiments, a treatment formulation comprises liquid state fermentation broth. In some embodiments, a treatment formulation comprises one or more solid carrier. In some embodiments, a treatment formulation comprises one or more adherent. In some embodiments, a treatment formulation comprises talc and mineral oil. In some embodiments, a treatment formulation comprises kaolin clay, a dispersant, and a surfactant. In some embodiments, a treatment formulation comprises a sugar.

In some embodiments, the synthetic composition additionally comprises one or more of a surfactant, a buffer, a tackifier, a microbial stabilizer, a fungicide, an anticomplex agent, an herbicide, a nematicide, an insecticide, a plant growth regulator, a rodenticide, a desiccant, a nutrient, an excipient, a wetting agent, a salt, and a polymer. In some embodiments, the polymer is a biodegradable polymer selected from the group consisting of alginate, agarose, agar, gelatin, polyacrylamide, chitosan, polyvinyl alcohol, and combinations thereof. In some embodiments, the biodegradable polymer is alginate and the alginate is sodium alginate or calcium alginate.

In some embodiments, the synthetic composition comprises one or more endophytes of the present invention and one or more chemical or biological agents capable of killing a pest of a plant, impeding the feeding and or growth and or reproduction of a pest of a plant, repelling a pest of a plant, and or reducing the severity or extent of infection of a plant host by a pest of a plant, including without limitation chemical or biological agents that are acetylcholinesterase (AChE) inhibitors, GABA-gated chloride channel blockers, sodium channel modulators, nicotinic acetylcholine receptor (nAChR) competitive modulators, nicotinic acetylcholine receptor (nAChR) allosteric modulators-Site I, Glutamate-gated chloride channel (GluCl) allosteric modulators, Chordotonal organ TRPV channel modulators, Nicotinic acetylcholine receptor (nAChR) channel blockers, Octopamine receptor agonists, Voltage-dependent sodium channel blockers, multi-site inhibitors, Ryanodine receptor modulators, chordotonal organ modulators (wherein the chordotonal organ modulator does not bind to the Nan-lav TRPV channel complex), GABA-gated chloride channel allosteric modulators, GABA-gated chloride channel allosteric modulators-Site II, nicotinic acetylcholine receptor (nAChR) Allosteric Modulators-Site II, Juvenile hormone mimics, Mite growth inhibitors affecting CHS1, Inhibitors of chitin biosynthesis affecting CHS1, Inhibitors of chitin biosynthesis-type 1, Moulting disruptors-Dipteran, Ecdysone receptor agonists, Inhibitors of acetyl COA carboxylase, Inhibitors of mitochondrial ATP synthase, Uncouplers of oxidative phosphorylation via disruption of the proton gradient, Mitochondrial complex III electron transport inhibitors, Mitochondrial complex I electron transport inhibitors, Mitochondrial complex IV electron transport inhibitors, Mitochondrial complex II electron transport inhibitors, Microbial disruptors of insect midgut membranes, Host-specific occluded pathogenic viruses, other active compounds (such as Azadirachtin, Benzoximate, Bromopropylate, Chinomethionat, Dicofol, Lime sulfur, Mancozeb, Pyridalyl, Sulfur, Chlorantraniliprole, Clothianidin, Tioxazafen, Fluopyram, Triticonazole), other active bacterial agents (such as certain Burkholderia strains including without limitation Burkholderia rinojenses, Wolbachia pipientis), other active fungal agents (such as Beauveria bassiana strains, Metarhizium anisopliae strain F52, Paecilomyces fumosoroseus Apopka strain 97), biological essence including synthetics or extracts or refined or unrefined oils (such as Dysphania ambrosioides near ambrosioides extract, fatty acid monoesters with glycerol or propanediol, neem oil), non-specific mechanical disruptors (such as Diatomaceous earth), or combinations thereof. Examples of AChE inhibitors include without limitation Carbamates (such as Alanycarb, Aldicarb, Bendiocarb, Benfuracarb, Butocarboxim, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiodicarb, Thiofanox, Triazamate, Trimethacarb, XMC, Xylylcarb) and Organophosphates (such as Acephate, Azamethiphos, Azinphos-ethyl, Azinphosmethyl, Cadusafos, Chlorethoxyfos, Chlorfenvinphos, Chlormephos, Chlorpyrifos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fosthiazate, Heptenophos, Imicyafos, Isofenphos, Isopropyl O-(methoxyaminothio-phosphoryl) salicylate, Isoxathion, Malathion, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-methyl, Profenofos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon, Vamidothion). Examples of GABA-gated chloride channel blockers include without limitation Cyclodiene Organochlorines (such as Chlordane, Endosulfan) and Phenylpyrazoles (Fiproles) (such as Ethiprole, Fipronil). Examples of sodium channel modulators include without limitation pyrethroids and pyrethrins (such as Acrinathrin, Allethrin, d-cis-trans Allethrin, d-trans Allethrin, Bifenthrin, Bioallethrin, Bioallethrin Scyclopentenyl isomer, Bioresmethrin, Cycloprothrin, Cyfluthrin, beta-Cyfluthrin, Cyhalothrin, lambda-Cyhalothrin, gamma-Cyhalothrin, Cypermethrin, alpha-Cypermethrin, beta-Cypermethrin, thetacypermethrin, zeta-Cypermethrin, Cyphenothrin, (1R)-trans-isomers], Deltamethrin, Empenthrin (EZ)-(1R)-isomers], Esfenvalerate, Etofenprox, Fenpropathrin, Fenvalerate, Flucythrinate, Flumethrin, tau-Fluvalinate, Halfenprox, Imiprothrin, Kadethrin, Permethrin, Phenothrin [(1R)-trans-isomer], Prallethrin, Pyrethrins (pyrethrum), Resmethrin, Silafluofen, Tefluthrin, Tetramethrin, Tetramethrin [(1R)-isomers], Tralomethrin, Transfluthrin) and Methoxychlor. Examples of nAChR competitive modulators include without limitation Neonicotinoids (such as Acetamiprid, Clothianidin, Dinotefuran, Imidacloprid, Nitenpyram, Thiacloprid, Thiamethoxam), nicotine, sulfoximines (such as Sulfoxaflor), Butenolides (such as Flupyradifurone), and Mesoionics (such as Triflumezopyrim). Examples of nAChR allosteric modulators-Site I include without limitation Spinosyns (such as Spinetoram, Spinosad). Examples of GluCl allosteric modulators include without limitation Avermectins and Milbemycins (such as Abamectin, Emamectin benzoate, Lepimectin, Milbemectin). Examples of multi-site inhibitors include without limitation Alkyl halides (such as Methyl bromide and other alkyl halides), Chloropicrin, Fluorides (such as Cryolite (Sodium aluminum fluoride), Sulfuryl fluoride), Borates (such as Borax, Boric acid, Disodium octaborate, Sodium borate, Sodium metaborate), Tartar emetic, Methyl isothiocyanate generators (such as Dazomet, Metam). Examples of chordotonal organ TRPV channel modulators include without limitation Pyridine azomethine derivatives (such as Pymetrozine, Pyrifluquinazon), and Pyropenes (such as Afidopyropen). Examples of juvenile hormone mimics include without limitation juvenile hormone analogues (such as Hydroprene, Kinoprene, Methoprene), fenoxycarb, and pyriproxyfen. Examples of mite growth inhibitors affecting CHS1 include without limitation Clofentezine, Diflovidazin, Hexythiazox, and Etoxazole. Examples of microbial disruptors of insect midgut membranes include without limitation Bacillus thuringiensis (such as Bacillus thuringiensis subsp. israelensis, Bacillus thuringiensis subsp. aizawai, Bacillus thuringiensis subsp. kurstaki, Bacillus thuringiensis subsp. tenebrionis, Bacillus thuringiensis strain EX297512) and the insecticidal proteins they produce (such as Cry1Ab, Cry1Ac, Cry1Fa, Cry1A.105, Cry2Ab, Vip3A, mCry3A, Cry3Ab, Cry3Bb, Cry34Ab1/Cry35Ab1) and Bacillus sphaericus. Examples of inhibitors of mitochondrial ATP synthase include without limitation Diafenthiuron, Organotin miticides (such as Azocyclotin, Cyhexatin, Fenbutatin oxide), Propargite, and Tetradifon. Examples of uncouplers of oxidative phosphorylation via disruption of the proton gradient include without limitation Pyrroles (such as Chlorfenapyr), Dinitrophenols, and Sulfluramid. Examples of nAChR channel blockers include without limitation Nereistoxin analogues (such as Bensultap, Cartap hydrochloride, Thiocyclam, Thiosultap-sodium). Examples of inhibitors of chitin biosynthesis affecting CHS1 include without limitation Benzoylureas (such as Bistrifluron, Chlorfluazuron, Diflubenzuron, Flucycloxuron, Flufenoxuron, Hexaflumuron, Lufenuron, Novaluron, Noviflumuron, Teflubenzuron, Triflumuron). Examples of inhibitors of chitin biosynthesis-type 1 include without limitation Buprofezin. Examples of moulting disruptors (Dipteran) include without limitation Cyromazine. Examples of ecdysone receptor agonists include without limitation Diacylhydrazines (such as Chromafenozide, Halofenozide, Methoxyfenozide, Tebufenozide). Examples of octopamine receptor agonists include without limitation Amitraz. Examples of mitochondrial complex III electron transport inhibitors include without limitation Hydramethylnon, Acequinocyl, Fluacrypyrim, and Bifenazate. Examples of mitochondrial complex I electron transport inhibitors include without limitation METI acaricides and insecticides such as Fenazaquin, Fenpyroximate, Pyridaben, Pyrimidifen, Tebufenpyrad, Tolfenpyrad) and Rotenone. Examples of voltage-dependent sodium channel blockers include without limitation Oxadiazines (such as Indoxacarb) and Semicarbazones (such as Metaflumizone). Examples of inhibitors of acetyl CoA carboxylase include without limitation Tetronic and Tetramic acid derivatives (such as Spirodiclofen, Spiromesifen, Spiropidion, Spirotetramat). Examples of mitochondrial complex IV electron transport inhibitors include without limitation Phosphides (Aluminium phosphide, Calcium phosphide, Phosphine, Zinc phosphide), Cyanides (such as Calcium cyanide, Potassium cyanide, Sodium cyanide). Examples of mitochondrial complex II electron transport inhibitors include without limitation Beta-ketonitrile derivatives (such as Cyenopyrafen, Cyflumetofen) and Carboxanilides (such as Pyflubumide). Examples of ryanodine receptor modulators include without limitation such as Diamides (such as Chlorantraniliprole, Cyantraniliprole, Cyclaniliprole Flubendiamide, Tetraniliprole). Examples of chordotonal organ modulators include without limitation Flonicamid. Examples of GABA-gated chloride channel allosteric modulators include without limitation Meta-diamides (Broflanilide) and Isoxazolines (such as Fluxametamide). Examples of nicotinic acetylcholine receptor (nAChR) Allosteric Modulators-Site II include without limitation GS-omega/kappa HXTX-Hv1a peptide.

In some embodiments, the synthetic composition comprises one or more endophytes of the present invention and one or chemical or biological agent capable of killing a pathogen of a plant, impeding the feeding and or growth and or reproduction of a pathogen of a plant, repelling a pathogen of a plant, and or reducing the severity or extent of infection of a plant host by a pathogen of a plant, including without limitation chemical or biological agents that are PhenylAmides fungicides (acylalanines, oxazolidinones, butyrolactones), hydroxy-(2-amino-) pyrimidines, heteroaromatics (such as isoxazoles, isothiazolones), carboxylic acids, Methyl-Benzimidazole-Carbamates (MBC) fungicides (such as thiophanates, benzimidazoles), N-phenyl carbamates, benzamides (such as toluamides, pyridinylmethyl-benzamides), thiazole carboxamide (such as ethylamino-thiazole-carboxamide), phenylureas, cyanoacrylates (such as aminocyanoacrylates), aryl-phenyl-ketones (such as benzophenone, benzoylpyridine), pyrimidinamines, pyrazole-MET1 (such as pyrazole-5-carboxamides), quinazoline, succinate-dehydrogenase inhibitors (SDHI) (such as phenyl-benzamides, phenyl-oxo-ethyl thiophene amide, pyridinyl-ethyl-benzamides, phenyl-cyclobutyl-pyridineamide, furan-carboxamides, oxathiin-carboxamides, thiazole-carboxamides, pyrazole-4-carboxamides, N-cyclopropyl-N-benzyl-pyrazole-carboxamides, N-methoxy-(phenyl-ethyl)-pyrazole-carboxamides, pyridine-carboxamides, pyrazine-carboxamides, pydiflumetofen, fluxapyroxad), quinone outside inhibitors (such as methoxy-acrylates, methoxy-acetamide, methoxy-carbamates, oximino-acetates, oximino-acetamides, oxazolidine-diones, dihydro-dioxazines, imidazolinones, benzyl-carbamates, tetrazolinones), quinone inside inhibitors (such as cyano-imidazole, sulfamoyl-triazole, picolinamides), uncouplers of oxidative phosphorylation (such as dinitrophenyl-crotonates, 2,6-dinitro-anilines), organo tin compounds (tri-phenyl tin compounds), thiophene-carboxamides, Quinone outside Inhibitor-stigmatellin binding type (such as triazolo-pyrimidylamine), anilino-pyrimidines, enopyranuronic acid antibiotic, hexopyranosyl antibiotic, glucopyranosyl antibiotic, tetracycline antibiotic, aza-naphthalenes (such as aryloxyquinoline, quinazolinone), phenylpyrroles, dicarboximides, phosphoro-thiolates, dithiolanes, aromatic hydrocarbons, chlorophenyls, nitroanilines, heteroaromatics (such as 1,2,4-thiadiazoles), carbamates, demethylation inhibitors (such as piperazines, pyridines, pyrimidines, imidazoles, triazoles, triazolinthiones), amines (such as morpholines, piperidines, spiroketal-amines), ketoreductase inhibitors (such as hydroxyanilides, amino-pyrazolinone), thiocarbamates, allylamines, polyoxins (such as peptidyl pyrimidine nucleoside), Carboxylic Acid Amides (such as cinnamic acid amides, valinamide carbamates, mandelic acid amides), melanin biosynthesis inhibitors-reductase (such as isobenzo-furanone, pyrrolo-quinolinone, triazolobenzo-thiazole), melanin biosynthesis inhibitors-dehydratase (such as cyclopropane-carboxamide, carboxamide, propionamide), melanin biosynthesis inhibitors-polyketide synthase (such as trifluoroethyl-carbamate), benzo-thiadiazole, benzisothiazole, thiadiazole-carboxamide, polysaccharides (such as laminarin), plant ethanol extracts (such as anthraquinones, resveratrol, extract from Reynoutria sachalinensis), phosphonates (such as ethyl phosphonates, fosetyl-Al, phosphorous acid and salts), isothiazole (such as isothiazolylmethyl ether), cyanoacetamide-oxime, phthalamic acids, benzotriazines, benzene-sulphonamides, pyridazinones, phenyl-acetamide, guanidines, thiazolidine (such as cyano-methylene-thiazolidines), pyrimidinone-hydrazones, 4-quinolyl-acetates, tetrazolyloximes, glucopyranosyl antibiotics, copper salts, sulphur, dithio-carbamates and relatives (such as amobam, ferbam, mancozeb, maneb, metiram, propineb, thiram, zinc thiazole, zineb, ziram), phthalimides, chloronitriles (phthalonitriles), sulfamides (such as dichlofluanid, tolylfluanid), bis-guanidines (such as guazatine, iminoctadine), triazines (such as anilazine), quinones (anthraquinones) (such as dithianon), quinoxalines (such as chinomethionat, quinomethionate), maleimide (such as fluoroimide), thiocarbamate (such as methasulfocarb), polypeptide (lectin) plant extracts (such as extract from the cotyledons of lupine plantlets), phenol and sesquiterpene and triterpenoid and coumarin plant extracts (such as extract from Swinglea glutinosa), terpene hydrocarbon and terpene alcohol and terpene phenol extracts plant extracts (such as extract from Melaleuca alternifolia, plant oils such as eugenol, geraniol, thymol mixtures thereof), Polyene (such as amphoteric macrolide antifungal antibiotic from Streptomyces natalensis or Streptomyces chattanoogensis), oxysterol binding protein homologue inhibition (piperidinyl-thiazole-isoxazolines), other active compounds (such as Fludioxonil, Mefenoxam, Sedaxane, Azoxystrobin, Thiabendazole, Ethaboxam, metalaxyl (such as without limitation metalaxyl-M), Trifloxystrobin, Myclobutanil, Acibenzolar-S-methyl, Metconazole, tolclofos-methyl, Fluopyram, Ipconazole, Oxathiapiprolin, Difenoconazole, Prothyoconazol, Tebuconazole, Pyraclostrobin, Fluxapyroxad, Triticonazole, Fluaxapyroxad), and combinations thereof.

In some embodiments, the synthetic composition comprises one or more endophytes of the present invention and one or more biological agents (for example bacterial or fungal agents) including, but not limited to, those agents capable of killing a pest or pathogen of a plant, impeding the feeding and or growth and or reproduction of a pest or pathogen of a plant, repelling a pest or pathogen of a plant, and or reducing the severity or extent of infection of a plant host by a pathogen or pest of a plant. The one or more bacterial or fungal agents may be living or dead (including without limitation by heat inactivation) bacteria or fungi, extracts and or metabolites of bacteria or fungi (including without limitation extracts and or metabolites in spent growth media), or combinations thereof. Non-limiting examples of biological agents include Trichoderma species including without limitation Trichoderma atroviride strain I-1237, Trichoderma atroviride strain LU132, Trichoderma atroviride strain SC1, Trichoderma atroviride strain SKT-1, Trichoderma atroviride strain 77B, Trichoderma asperellum strain T34, Trichoderma asperellum strain kd, Trichoderma harzianum strain T-22, Trichoderma virens strain G-41; Clonostachys species including without limitation Gliocladium catenulatum strain J1446, Clonostachys rosea strain CR-7; Coniothyrium species including without limitation Coniothyrium minitans strain CON/M/91-08; Talaromyces species including without limitation Talaromyces flavus strain SAY-Y-94-01; Saccharomyces species including without limitation Saccharomyces cerevisae strain LAS02; Bacillus species including without limitation Bacillus amyloliquefaciens strain QST713, Bacillus amyloliquefaciens strain FZB24, Bacillus amyloliquefaciens strain MBI600, Bacillus amyloliquefaciens strain D747, Bacillus amyloliquefaciens strain F727, Bacillus amyloliquefaciens strain AT-332, Bacillus amyloliquefaciens strain MBI 600 Bacillus mycoides isolate J, Bacillus subtilis strain AFS032321, Bacillus subtilis strain Y1336, Bacillus subtilis strain MBI 600, Bacillus subtilis strain HAI-0404, Bacillus firmus I-1582); Pseudomonas species including without limitation Pseudomonas chlororaphis strain AFS009; Streptomyces species including without limitation Streptomyces griseovirides strain K61, Streptomyces lydicus strain WYEC108; Penicillium species such as Penicillium bilaiae, Penicillium bilaiae; and Pasteuria species including without limitation Pasteuria nishizawae Pn1).

In some embodiments, one or more endophytes of the present invention and one or chemical or biological agents described herein are present in a synthetic composition at a weight ratio of between 1000:1 and 1:1000, 100:1 and 1:100, or 10:1 and 1:10.

In some embodiments, the synthetic composition may be stored at between 0 degrees Celsius and 4 degrees Celsius for 1 week with less than 1 log loss of CFU of the one or more endophytes. In some embodiments, the synthetic composition may be stored at between 4.1 degrees Celsius and 20 degrees Celsius for 1 week with less than 1 log loss of CFU of the one or more endophytes. In some embodiments, the synthetic composition may be stored at between 20.1 degrees Celsius and 35 degrees Celsius for 1 week with less than 1 log loss of CFU of the one or more endophytes.

In some embodiments, a synthetic composition comprises one or more endophytes heterologously disposed into a treatment formulation. In some embodiments, the treatment formulation is a liquid. In some embodiments, an endophyte heterologously disposed into a liquid treatment formulation is present in the treatment formulation at a titer of at least 1E7, 1E8, 1E9, 1E10 CFU/mL. In some embodiments, an endophyte heterologously disposed into a liquid treatment formulation is present in the treatment formulation at a titer of between 1E7 and 1E9 CFU/mL. In some embodiments, an endophyte heterologously disposed into a liquid treatment formulation is present in the treatment formulation at a titer of between 1E7 and 1E9 CFU/mL. In some embodiments, an endophyte heterologously disposed into a liquid treatment formulation is present in the treatment formulation at a titer of about 3E9 CFU/mL. In some embodiments, the treatment formulation is a powder, for example a wettable powder or a flowable powder. In some embodiments, an endophyte heterologously disposed into powder treatment formulation is present in the treatment formulation at a titer of at least 1E7, 1E8, 1E9, 1E10, 1E11, 1E12 CFU/g. In some embodiments, an endophyte heterologously disposed into a powder treatment formulation is present in the treatment formulation at a titer of between 1E7 and 1E11 CFU/mL. In some embodiments, an endophyte heterologously disposed into a powder treatment formulation is present in the treatment formulation at a titer of between 1E8 and 1E10 CFU/mL. In some embodiments, an endophyte heterologously disposed into a powder treatment formulation is present in the treatment formulation at a titer of about 3E9 CFU/mL. In some embodiments, an endophyte heterologously disposed into a powder treatment formulation is present in the treatment formulation at a titer of about 2E10 CFU/mL. In some embodiments, an endophyte heterologously disposed into a wettable powder treatment formulation is present in the treatment formulation at a titer of between 1E9 and 1E11 CFU/mL. In some embodiments, an endophyte heterologously disposed into a flowable powder treatment formulation is present in the treatment formulation at a titer of between 1E9 and 1E10 CFU/mL.

In yet another aspect, described herein are methods of enriching a population of beneficial endophytes, comprising determining the presence or abundance of one or more endophytes in a plant element, growth medium or growth environment, wherein the one or more endophytes comprise at least one polynucleotide sequence that is at least 97% identical to one or more of SEQ ID NOs. 1-83, and combinations thereof. In some embodiments, the presence or abundance of one or more endophytes is determined relative to a reference plant element, growth medium or growth environment. In some embodiments, the one or more endophytes are not present in the reference plant element, growth medium or growth environment. In some embodiments, the one or more endophytes are less abundant in the reference plant element, growth medium or growth environment. In some embodiments, the presence or abundance of one or more endophytes is determined in a plant element and modulation of one or more traits of agronomic importance is inferred from the presence or amount of the one or more endophytes in the plant element. In some embodiments, the presence or abundance of one or more endophytes is determined in a growth medium and the capacity of the growth medium to modulate one or more trait of agronomic importance in a plant element planted therein is inferred from the presence or amount of the one or more endophytes in the growth medium. In some embodiments, the presence or abundance of one or more endophytes is determined in a growth environment and the capacity of the growth environment to modulate one or more trait of agronomic importance in a plant element grown therein is inferred from the presence or amount of the one or more endophytes in the growth environment. In some embodiments, the presence or abundance of one or more endophytes is determined by polymerase chain reaction, fluorescence in situ hybridization, or isothermal amplification. In some embodiments, the method further comprises the step of isolating the selected endophytes.

In some embodiments, a plurality of nucleic acid probes are used to determine the presence or abundance of one or more endophytes in a plant element, growth medium or growth environment, wherein the plurality comprises complementary or reverse complementary sequences to a region of at least 10 contiguous nucleotides within one or more polynucleotide sequences selected from the group consisting of SEQ ID NOs. 1-83, and combinations thereof. In some embodiments, the complementary or reverse complementary region comprises at least 20 contiguous nucleotides. In some embodiments, the complementary or reverse complementary region comprises at least 30 contiguous nucleotides. In some embodiments, the complementary or reverse complementary region comprises at least 40 contiguous nucleotides. In some embodiments, the plurality of nucleic acid probes are single-stranded DNA. In some embodiments, the plurality of nucleic acid probes are attached to one or more solid supports. In some embodiments, the plurality of nucleic acid probes are attached to a plurality of beads. In some embodiments, the plurality of nucleic acid probes are attached to a contiguous solid support.

In some embodiments, the plant element is a monocot. In some embodiments, the monocot is a cereal. In some embodiments, the cereal is selected from the group consisting of wheat, rice, barley, buckwheat, rye, millet, oats, corn, sorghum, triticale and spelt. In some embodiments, the cereal is wheat.

In some embodiments, the plant element is a dicot. In some embodiments, the dicot is selected from the group consisting of cotton, tomato, lettuce, peppers, cucumber, endive, melon, potato, cannabis, and squash. In some embodiments, the dicot is a legume. In some embodiments, the legume is soy, peas or beans.

In some embodiments, the plant element is a whole plant, seedling, meristematic tissue, ground tissue, vascular tissue, dermal tissue, seed, leaf, root, shoot, stem, flower, fruit, stolon, bulb, tuber, corm, keikis, shoot, or bud. In some embodiments, the plant element is a seed.

In some embodiments, the trait of agronomic importance is selected from the group consisting of drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, biotic stress tolerance, improved disease resistance, yield improvement, health enhancement, vigor improvement, decreased necrosis, decreased chlorosis, decreased area of necrotic tissue, decreased area of chlorotic tissue, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof. In some embodiments, the trait of agronomic importance is biotic stress tolerance. In some embodiments, the trait of agronomic importance is improved nutrient use efficiency. In some embodiments, the trait of agronomic importance is drought tolerance.

In some embodiments, the one or more endophytes is a member of the Class Gammaproteobacteria. In some embodiments, the one or more endophytes is a member of the Order Enterobacterales. In some embodiments, the one or more endophytes is a member of the Family Enterobacteriaceae. In some embodiments, the one or more endophytes is a member of the Genus Kosakonia. In some embodiments, the one or more endophytes is a Kosakonia cowanii.

In some embodiments, the one or more endophytes comprises at least 2 endophytes. In some embodiments, the one or more endophytes comprises at least 3 endophytes. In some embodiments, the one or more endophytes comprises at least 4 endophytes. In some embodiments, the one or more endophytes comprises at least 5 endophytes. In some embodiments, the one or more endophytes comprises at least 10 endophytes.

In some embodiments, the one or more endophytes are encapsulated in polymeric beads. In some embodiments, the polymeric beads are less than 500 μm in diameter at their widest point. In some embodiments, the polymeric beads are less than 200 μm in diameter at their widest point. In some embodiments, the polymeric beads are less than 100 μm in diameter at their widest point. In some embodiments, the polymeric beads are less than 50 μm in diameter at their widest point. In some embodiments, the polymeric beads' average diameter at their widest point is between 500 μm and 250 μm. In some embodiments, the polymeric beads' average diameter at their widest point is between 249 μm and 100 μm. In some embodiments, the polymeric beads' average diameter at their widest point is between 100 μm and 50 μm.

In some embodiments, the one or more endophytes are encapsulated in inorganic or mineral particles. In some embodiments, the inorganic or mineral particles are silica, clay, talc, sand, silt, and magnetite. In some embodiments, the one or more endophytes are encapsulated in organic matter particles. In some embodiments, the organic matter particles are urea, humus, active carbons, proteins, biochar, carbohydrate, and lipids.

### BRIEF DESCRIPTION OF THE DRAWINGS

FIG. 1A, FIG. 1B, FIG. 2, FIG. 3A, FIG. 3B, and FIG. 4 show exemplary images of sterile plates; each was inoculated with a plug of fungal pathogen placed in the center of the plate, and surrounded by four autoclaved wheat seeds treated with MIC-70076 or chemical fungicide or untreated controls. FIG. 1A, FIG. 1B, FIG. 2, FIG. 3A, FIG. 3B, and FIG. 4 contain the following numeric annotations: 101 indicates autoclaved wheat seeds that are not surrounded by pathogen hyphae, 102 indicates autoclaved wheat seeds that are in contact with pathogen hyphae, 103 indicates the outer edge of the pathogen colony, 104 indicates the position of inoculation with a plug of pathogen, 105 indicates a region of visible biofilm formation.

FIG. 1A, FIG. 1B, and FIG. 2 show exemplary images of plates inoculated with Dreschlera tritici-repentis.

FIG. 1A shows an exemplary image of a plate inoculated with Dreschlera tritici-repentis and four autoclaved wheat seeds treated with MIC-70076 (0.65 ml/kg).

FIG. 1A shows that a visible biofilm has formed around the treated seed 105, a halo of inhibition has formed around each seed, and the area of Dreschlera tritici-repentis growth on the plate is limited. FIG. 1B shows an exemplary image of a plate inoculated with Dreschlera tritici-repentis and four autoclaved wheat seeds treated with chemical fungicides Thiram and Carbendazim. FIG. 1B also shows the area of Dreschlera tritici-repentis growth on the plate is limited.

FIG. 2 shows an exemplary image of a plate inoculated with Dreschlera tritici-repentis and four wheat seeds not treated with any endophyte or chemical fungicide. All autoclaved wheat seeds shown in FIG. 2 are in contact with pathogen hyphae.

FIG. 3A shows an exemplary image of a plate inoculated with Bipolaris sorokiniana and autoclaved four wheat seeds treated with MIC-70076 (0.65 ml/kg). FIG. 3A shows that a region of inhibition has formed around each seed, and the area of Bipolaris sorokiniana growth on the plate is limited.

FIG. 3B shows an exemplary image of a plate inoculated with Bipolaris sorokiniana and four wheat seeds treated with chemical fungicides Thiram and Carbendazim. FIG. 3B also shows the area of Bipolaris sorokiniana is not significantly reduced relative to the untreated control in FIG. 4. The Thiram and Carbendazim treatment did not reduce the area of Bipolaris sorokiniana growth on the plate. All autoclaved wheat seeds shown in FIG. 3B are in contact with pathogen hyphae.

FIG. 4 shows an exemplary image of a plate inoculated with Bipolaris sorokiniana and four autoclaved wheat seeds not treated with any endophyte or chemical fungicide. All autoclaved wheat seeds shown in FIG. 4 are in contact with pathogen hyphae.

FIG. 5 shows the average colony diameter (in mm) of plates inoculated with Bipolaris sorokiniana by days of incubation and seed treatment. Three seed treatments are shown, from left to right: results from plates containing seeds treated with 0.65 ml/kg MIC-70076, seeds treated with chemical fungicides Thiram and Carbendazim, and untreated seeds. For each of the three treatments, bars represent the diameter in millimeters of the Bipolaris sorokiniana colony, from left to right, after 2, 4, 6, and 8 days of incubation.

FIG. 6 shows the average colony diameter (in mm) of plates inoculated with Dreschlera tritici-repentis by days of incubation and seed treatment. Three seed treatments are shown, from left to right: results from plates containing seeds treated with 0.65 ml/kg MIC-70076, seeds treated with chemical fungicides Thiram and Carbendazim, and untreated seeds. For each of the three treatments, bars represent the diameter in millimeters of the Dreschlera tritici-repentis colony, from left to right, after 2, 4, 6, and 8 days of incubation.

### DETAILED DESCRIPTION

Terms used in the claims and specification are defined as set forth below unless otherwise specified.

It must be noted that, as used in the specification and the appended claims, the singular forms “a,” “an” and “the” include plural referents unless the context clearly dictates otherwise.

This invention relates to methods and compositions for improving plant health. The present invention includes methods for improving plant health, as well as synthetic compositions comprising endophytes capable of improving plant health, and nucleic acid probes and nucleic acid detection kits that may be used to identify endophytes of the present invention.

“Plant health” is demonstrated by the improvement of a trait of agronomic importance in a plant or plant element as compared to a reference plant or plant element. A trait of agronomic importance includes, but is not limited to, drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, biotic stress tolerance, increased disease resistance, yield improvement, health enhancement, vigor improvement, decreased necrosis, decreased chlorosis, decreased area of necrotic tissue, decreased area of chlorotic tissue, decreased pathogen load of tissues, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof. The phrase “biotic stress” refers to a growth environment comprising one or more pests or pathogens. Pests can be nematodes and/or insects. In some embodiments, a pest is of an order Lepidoptera, Hemiptera, Tylenchida/Rhabditida, Dorylaimida, Trichinellida, or Triplonchida. In some embodiments, a pest is of a genera Chrysodeixis, Trichoplusia, Nezara, Lygus, Aphis, Belonolaimus, Xiphenema, Trichodorus, Pratylenchus, Aphelenchoides, Meloidogyne, or Rotylenchulus. Pathogens can be fungal, viral, protist, or bacterial pathogens, for example pathogens of vertebrates or plants. In some embodiments, a pathogen is of a genera Pythium, Rhizoctonia, Phytophthora, Fusarium, Alternaria, Stagonospora, Aspergillus, Magnaporthe, Biopolaris, Dreschlera, Botrytis, Puccinia, Blumeria, Erysiphe, Leveillula, Mycosphaerella, or Colletotrichum.

“Biomass” means the total mass or weight (fresh or dry), at a given time (for example, age or stage of development), of a plant tissue, plant tissues, an entire plant, or population of plants. The term may also refer to all the plants or species in the community (“community biomass”).

An “increased yield” can refer to any increase in seed or fruit biomass; or seed, seed pod or ear, or fruit number per plant; or seed or fruit weight; or seed or fruit size per plant or unit of production area, e.g. acre or hectare. For example, increased yield of seed or fruit biomass may be measured in units of bushels per acre, pounds per acre, tons per acre, or kilos per hectare. An increased yield can also refer to an increased production of a component of, or product derived from, a plant or plant element or of a unit of measure thereof. For example, increased carbohydrate yield of a grain or increased oil yield of a seed. Typically, where yield indicates an increase in a particular component or product derived from a plant, the particular characteristic is designated when referring to increased yield, e.g., increased oil or grain yield or increased protein yield or seed size.

“Nutrition enhancement” refers to modulation of the presence, abundance or form of one or more substances in a plant element, wherein the modulation of the one or more substances provides a benefit to other organisms that consume or utilize said plant element.

Synthetic compositions and methods of use described herein may improve plant health by providing an improved benefit or tolerance to a plant that is of at least 0.1%, at least 0.5%, at least 1%, at least 2%, at least 3%, between 3% and 5%, at least 5%, between 5% and 10%, at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with a reference plant. A “reference plant”, “reference plant element”, “reference agricultural plant” or “reference seed” means a similarly situated plant or seed of the same species, strain, or cultivar to which a treatment, formulation, composition or endophyte preparation as described herein is not administered/contacted. A reference plant, therefore, is identical to the treated plant except for the presence of the active ingredient to be tested and can serve as a control for detecting the effects of the treatment conferred to the plant. A plurality of reference plants may be referred to as a “reference population”.

In some embodiments, one or more endophytes and or one or more compounds produced by one or more endophytes are heterologously disposed on a plant element in an effective amount to improve plant health. In some embodiments, an improvement of plant health is measured by an increase in a trait of agronomic importance, for example root length or yield. In some embodiments, an improvement of subject health is measured by a decrease in a trait of importance, for example necrosis or chlorosis. In some embodiments, improved plant health is demonstrated by an improvement of a trait of agronomic importance or tolerance in a treated plant by at least 0.1%, at least 0.5%, at least 1%, at least 2%, at least 3%, between 3% and 5%, at least 5%, between 5% and 10%, at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant or plant element. In some embodiments, improved plant health is demonstrated by a “win rate” a proportion of experimental trials showing an improvement of a trait of agronomic importance or tolerance in a treated plant relative as compared to a reference plant or plant element. In some embodiments the win rate is at least 10%, at least 20%, at least 30%, at least 40%, at least 50%, at least 60%, at least 70%, at least 75%, at least 80% or more.

An “effective amount” of one or more endophytes is the amount capable of improving trait of agronomic importance or tolerance by at least 0.1%, at least 0.5%, at least 1%, at least 2%, at least 3%, between 3% and 5%, at least 5%, between 5% and 10%, at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant element not further comprising said endophyte. In some embodiments, an effective amount of treatment comprising an endophyte is at least 10 CFU per unit of plant element, at least 10\hat{ }2 CFU per unit of plant element, between 10\hat{ }2 and 10\hat{ }3 CFU per unit of plant element, at least about 10\hat{ }3 CFU per unit of plant element, between 10\hat{ }3 and 10\hat{ }4 CFU per unit of plant element, at least about 10\hat{ }4 CFU per unit of plant element, between 10\hat{ }4 and 10\hat{ }5 CFU per unit of plant element, at least about 10\hat{ }5 CFU, between 10\hat{ }5 and 10\hat{ }6 CFU per unit of plant element, at least about 10\hat{ }6 CFU per unit of plant element, between 10\hat{ }6 and 10\hat{ }7 CFU per unit of plant element, at least about 10\hat{ }7 CFU per unit of plant element, between 10\hat{ }7 and 10\hat{ }8 CFU per unit of plant element, or even greater than 10\hat{ }8 CFU per unit of plant element. A unit of a plant element may be an individual plant element, e.g. an individual seed, or a unit of area surface area of a plant element, e.g. a square inch of leaf tissue, or unit of surface area of a plant element, e.g. a cubic centimeter of root.

The methods and compositions of the present invention are broadly applicable to cultivated plants, particularly plants that are cultivated by humans for food, feed, fiber, fuel, and/or industrial purposes. In some embodiments, plants (including seeds and other plant elements) are monocots or dicots. In some embodiments, plants used in the methods and compositions of the present invention include, but are not limited to: agricultural row, agricultural grass plants or other field crops: wheat, rice, barley, buckwheat, beans (for example: soybean, snap, dry), corn (for example: grain, seed, sweet corn, silage, popcorn, high oil), canola, peas (for example: dry, succulent), peanuts, safflower, sunflower, alfalfa hay, forage and cover crops (for example: alfalfa, clover, vetch, and trefoil), berries and small fruits (for example: blackberries, blueberries, currants, elderberries, gooseberries, huckleberries, loganberries, raspberries, strawberries, bananas and grapes), bulb crops (for example: garlic, leeks, onions, shallots, and ornamental bulbs), citrus fruits (for example: citrus hybrids, grapefruit, kumquat, lines, oranges, and pummelos), cucurbit vegetables (for example: cucumbers, melons, gourds, pumpkins, and squash), flowers (for example: ornamental, horticultural flowers including roses, daisies, tulips, freesias, carnations, heather, lilies, irises, orchids, snapdragons, and ornamental sunflowers), bedding plants, ornamentals, fruiting vegetables (for example: eggplant, sweet and hot peppers, tomatillos, and tomatoes), herbs, spices, mints, hydroponic crops (for example: cucumbers, tomatoes, lettuce, herbs, and spices), leafy vegetables and cole crops (for example: arugula, celery, chervil, endive, fennel, lettuce including head and leaf, parsley, radicchio, rhubarb, spinach, Swiss chard, broccoli, Brussels sprouts, cabbage, cauliflower, collards, kale, kohlrabi, and mustard greens), asparagus, legume vegetable and field crops (for example: snap and dry beans, lentils, succulent and dry peas, and peanuts), pome fruit (for example: pears and quince), root crops (for example: beets, sugar beets, red beets, carrots, celeriac, chicory, horseradish, parsnip, radish, rutabaga, salsify, and turnips), deciduous trees (for example: maple and oak), evergreen trees (for example: pine, cedar, hemlock and spruce), small grains (for example: rye, wheat including spring and winter wheat, millet, oats, barley including spring and winter barley, and spelt), stone fruits (for example: apricots, cherries, nectarines, peaches, plums, and prunes), tree nuts (for example: almonds, beech nuts, Brazil nuts, butternuts, cashews, chestnuts, filberts, hickory nuts, macadamia nuts, pecans, pistachios, and walnuts), and tuber crops (for example: potatoes, sweet potatoes, yams, artichoke, cassava, and ginger). In a particular embodiment, the agricultural plant is selected from the group consisting of rice (Oryza sativa and related varieties), soy (Glycine max and related varieties), wheat (Triticum aestivum and related varieties), oats (Avena sativa and related varieties), barley (Hordeum vulgare and related varieties), corn (Zea mays and related varieties), peanuts (Arachis hypogaea and related varieties), canola (Brassica napus, Brassica rapa and related varieties), coffee (Coffea spp.), cocoa (Theobroma cacao), melons, and tomatoes (Solanum lycopsersicum and related varieties).

Plant health may be improved by treatment of a plant or plant element. A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, keikis, shoot, or bud.

Plant health may be improved by treatment with a composition of the present invention, in particular compositions of the present invention comprising one or more endophytes. An “endophyte” is an organism capable of living on a plant element (e.g., rhizoplane or phyllosphere) or within a plant element, or on a surface in close physical proximity with a plant element, e.g., the phyllosphere and rhizosphere including soil surrounding roots. A “beneficial” endophyte does not cause disease or harm the host plant otherwise. Endophytes can occupy the intracellular or extracellular spaces of plant tissue, including the leaves, stems, flowers, fruits, seeds, or roots. An endophyte can be, for example, a bacterial or fungal organism, and can confer a beneficial property to the host plant such as an increase in yield, biomass, resistance, or fitness. An endophyte can be a fungus or a bacterium. As used herein, the term “microbe” is sometimes used to describe an endophyte. As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is an endophyte, for example a bacterial or fungal endophyte, which is capable of living within a plant.

The term “isolated” is intended to specifically reference an organism, cell, tissue, polynucleotide, or polypeptide that is removed from its original source and purified from additional components with which it was originally associated. For example, an endophyte may be considered isolated from a seed if it is removed from that seed source and purified so that it is isolated from one or more additional components with which it was originally associated. Similarly, an endophyte may be removed and purified from a plant or plant element so that it is isolated and no longer associated with its source plant or plant element.

As used herein, an isolated strain of a microbe is a strain that has been removed from its natural milieu. “Pure cultures” or “isolated cultures” are cultures in which the organisms present are only of one strain of a particular genus and species. This is in contrast to “mixed cultures,” which are cultures in which more than one genus and/or species of microorganism are present. As such, the term “isolated” does not necessarily reflect the extent to which the microbe has been purified. A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants. Further, as used herein, a “biologically pure” strain is intended to mean the strain was separated from materials with which it is normally associated in nature. A strain associated with other strains, or with compounds or materials that it is not normally found with in nature, is still defined as “biologically pure.” A monoculture of a particular strain is, of course, “biologically pure.” As used herein, the term “enriched culture” of an isolated microbial strain refers to a microbial culture that contains more than 50%, 60%, 70%, 80%, 90%, or 95% of the isolated strain.

A “population” of endophytes, or an “endophyte population”, refers to one or more endophytes that share a common genetic derivation, e.g., one or more propagules of a single endophyte, i.e., endophytes grown from a single picked colony. In some embodiments, a population refers to endophytes of identical taxonomy. In some cases, a population of endophytes refers to one or more endophytes of the same genus. In some cases, a population of endophytes refers to one or more endophytes of the same species or strain.

A “plurality of endophytes” means two or more types of endophyte entities, e.g., of bacteria or fungi, or combinations thereof. In some embodiments, the two or more types of endophyte entities are two or more individual endophytic organisms, regardless of genetic derivation or taxonomic relationship. In some embodiments, the two or more types of endophyte entities are two or more populations of endophytes. In other embodiments, the two or more types of endophyte entities are two or more species of endophytes. In yet other embodiments, the two or more types of endophyte entities are two or more genera of endophytes. In yet other embodiments, the two or more types of endophyte entities are two or more families of endophytes. In yet other embodiments, the two or more types of endophyte entities are two or more orders of endophytes. In yet other embodiments, the two or more types of endophyte entities are two or more classes of endophytes. In yet other embodiments, the two or more types of endophyte entities are two or more phyla of endophytes. In some embodiments, a plurality refers to three or more endophytes, either distinct individual organisms or distinct members of different genetic derivation or taxa. In some embodiments, a plurality refers to four or more either distinct individual endophytic organisms or distinct members of different genetic derivation or taxa. In some embodiments, a plurality refers to five or more, ten or more, or an even greater number of either distinct individual endophytic organisms or distinct members of different genetic derivation or taxa. In some embodiments, the term “consortium” or “consortia” may be used as a collective noun synonymous with “plurality”, when describing more than one population, species, genus, family, order, class, or phylum of endophytes.

In some embodiments, a treatment may comprise a modified microbe or plant or plant element. A microbe or plant or plant element is “modified” when it comprises an artificially introduced genetic or epigenetic modification. In some embodiments, the modification is introduced by genome engineering or genome editing technology. In some embodiments, genome engineering or editing utilizes non-homologous end joining (NHEJ), homology directed repair (HDR), or combinations thereof. In some embodiments, genome engineering or genome editing is carried out with a Class I or Class II clustered regulatory interspaced short palindromic repeats (CRISPR) system. In some embodiments, the CRISPR system is CRISPR/Cas9. In some embodiments, the CRISPR system is CRISPR/Cpf1. In some embodiments, the modification is introduced by a targeted nuclease. In some embodiments, targeted nucleases include, but are not limited to, transcription activator-like effector nuclease (TALEN), zinc finger nuclease (ZNF), Cas9, Cas9 variants, Cas9 homologs, Cpf1, Cpf1 variants, Cpf1 homologs, and combinations thereof. In some embodiments, the modification is an epigenetic modification. In some embodiments, the modification is introduced by treatment with a DNA methyltransferase inhibitor such as 5-azacytidine, or a histone deacetylase inhibitor such as 2-amino-7-methoxy-3H-phenoxazin-3-one. In some embodiments, the modification is introduced via tissue culture. In some embodiments, a modified microbe or plant or plant element comprises a transgene.

As used herein, the term “bacterium” or “bacteria” refers in general to any prokaryotic organism and may reference an organism from either Kingdom Eubacteria (Bacteria), Kingdom Archaebacteria (Archaea), or both. In some cases, bacterial genera have been reassigned due to various reasons (such as, but not limited to, the evolving field of whole genome sequencing), and it is understood that such nomenclature reassignments are within the scope of any claimed genus.

As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. With the development of genomic sequencing, it became evident that taxonomic classification based on molecular phylogenetics did not align with morphological-based nomenclature (Shenoy B D, Jeewon R, Hyde K D. Impact of DNA sequence-data on the taxonomy of anamorphic fungi. Fungal Diversity 26 (10) 1-54. 2007). Systematics experts have not aligned on common nomenclature for all fungi, nor are all existing databases and information resources inclusive of updated taxonomies. As such, many fungi provided herein may be described by their anamorph form, but it is understood that based on identical genomic sequencing, any pleomorphic state of that fungus may be considered to be the same organism. In some cases, fungal genera have been reassigned due to various reasons, and it is understood that such nomenclature reassignments are within the scope of any claimed genus.

The degree of relatedness between microbes may be inferred from the sequence similarity of one or more homologous polynucleotide sequences of the microbes. In some embodiments, the one or more homologous polynucleotide sequences are marker genes. As used herein, the term “marker gene” refers to a conserved genomic region comprising sequence variation among related organisms. Examples of marker genes that may be used for the present invention, include but are not limited to: 16S ribosomal RNA gene (“16S”), internal transcribed spacer (“ITS”); fusA gene; largest subunit of RNA polymerase II (“RPB1”); second largest subunit of RNA polymerase II (“RPB2”); beta-tubulin or tubulin (“BTUB2” or “TUB2”); phosphoglycerate kinase (“PGK”); actin (“ACT”); long subunit rRNA gene (“LSU”); small subunit rRNA gene (“SSU”), 60S ribosomal protein L 10 (“60S_L10_L1”), atpD, Calmodulin (“CMD”), GDP gene (“GPD1_2”), etc.

The terms “sequence similarity”, “identity”, “percent identity”, “percent sequence identity” or “identical” in the context of polynucleotide sequences refer to the nucleotides in the two sequences that are the same when aligned for maximum correspondence. There are different algorithms known in the art that can be used to measure nucleotide sequence identity. Nucleotide sequence identity can be measured by a local or global alignment, preferably implementing an optimal local or optimal global alignment algorithm. For example, a global alignment may be generated using an implementation of the Needleman-Wunsch algorithm (Needleman, S. B. & Wunsch, C. D. (1970) Journal of Molecular Biology. 48 (3): 443-53). For example, a local alignment may be generated using an implementation of the Smith-Waterman algorithm (Smith T. F & Waterman, M. S. (1981) Journal of Molecular Biology. 147 (1): 195-197). Optimal global alignments using the Needleman-Wunsch algorithm and optimal local alignments using the Smith-Waterman algorithm are implemented in USEARCH, for example USEARCH version v8.1.1756_i86osx32.

A gap is a region of an alignment wherein a sequence does not align to a position in the other sequence of the alignment. A terminal gap is a region beginning at the end of a sequence in an alignment wherein the nucleotide in the terminal position of that sequence does not correspond to a nucleotide position in the other sequence of the alignment and extending for all contiguous positions in that sequence wherein the nucleotides of that sequence do not correspond to a nucleotide position in the other sequence of the alignment. An internal gap is a gap in an alignment which is flanked on the 3′ and 5′ end by positions wherein the aligned sequences are identical. In global alignments, terminal gaps are discarded before identity is calculated. For both local and global alignments, internal gaps are counted as differences.

In some embodiments, the nucleic acid sequence to be aligned is a complete gene. In some embodiments, the nucleic acid sequence to be aligned is a gene fragment. In some embodiments, the nucleic acid sequence to be aligned is an intergenic sequence. In a preferred embodiment, inference of homology from a sequence alignment is made where the region of alignment is at least 85% of the length of the query sequence.

The term “substantial homology” or “substantial similarity,” when referring to a polynucleotide sequence or fragment thereof, indicates that, when optimally aligned with appropriate nucleotide insertions or deletions with another polynucleotide sequence (or its complementary strand), there is nucleotide sequence identity in at least about 76%, 80%, 85%, or at least about 90%, or at least about 95%, 96%, at least 97%, 98%, 99% or 100% of the positions of the alignment, wherein the region of alignment is at least about 50%, 60%, 70%, 75%, 85%, or at least about 90%, or at least about 95%, 96%, 97%, 98%, 99% or 100% of the length of the query sequence. In a preferred embodiment, the region of alignment contains at least 100 positions inclusive of any internal gaps. In some embodiments, the region of alignment comprises at least 100 nucleotides of the query sequence. In some embodiments, the region of alignment comprises at least 200 nucleotides of the query sequence. In some embodiments, the region of alignment comprises at least 300 nucleotides of the query sequence. In some embodiments, the region of alignment comprises at least 400 nucleotides of the query sequence. In some embodiments, the region of alignment comprises at least 500 nucleotides of the query sequence. In some embodiments, the terminal nucleotides are trimmed from one or both ends of the sequence prior to alignment. In some embodiments, at least the terminal 10, 15, 20, 25, 30, between 20-30, 35, 40, 45, 50, between 25-50 nucleotides are trimmed from the sequence prior to alignment.

### Synthetic Compositions for Improving Plant Health

In some embodiments, a synthetic composition comprises one or more endophytes capable of improving plant health. A “synthetic composition” comprises one or more endophytes combined by human endeavor with a heterologously disposed plant element or a treatment formulation, said combination which is not found in nature. In some embodiments, a synthetic composition comprises one or more plant elements or formulation components combined by human endeavor with an isolated, purified endophyte composition. In some embodiments, synthetic composition refers to a plurality of endophytes in a treatment formulation comprising additional components with which said endophytes are not found in nature. An endophyte is “heterologously disposed” when mechanically or manually applied, artificially inoculated or disposed onto or into a plant element, seedling, plant or onto or into a plant growth medium or onto or into a treatment formulation so that the endophyte exists on or in the plant element, seedling, plant, plant growth medium, or formulation in a manner not found in nature prior to the application of the treatment, e.g., said combination which is not found in nature in that plant variety, at that time in development, in that tissue, in that abundance, or in that growth condition (for example, drought, flood, cold, nutrient deficiency, etc.).

A “treatment formulation” refers to one or more compositions that facilitate the stability, storage, and/or application of one or more endophytes. Treatment formulations may comprise any one or more agents such as: a surfactant, a buffer, a tackifier, a microbial stabilizer, an antimicrobial, a fungicide, an anticomplex agent, an herbicide, a nematicide, an insecticide, a plant growth regulator, a rodenticide, a desiccant, a nutrient, an excipient, a wetting agent, a salt, a polymer. As used herein as a noun, a “treatment” may comprise one or more endophytes.

In some embodiments, a treatment formulation may comprise one or more polymeric beads comprising one or more endophytes. In some embodiments, a treatment formulation may consist of one or more polymeric beads comprising one or more endophytes. A polymeric bead may contain a biodegradable polymer such as alginate, agarose, agar, gelatin, polyacrylamide, chitosan, and polyvinyl alcohol. In some embodiments, the polymeric beads are less than 500 μm in diameter at their widest point. In some embodiments, the polymeric beads' average diameter at their widest point is between 500 μm and 250 μm, between 249 μm and 100 μm, 100 μm or less, between 100 μm and 50 μm, or 50 μm or less.

In some embodiments, an “agriculturally compatible carrier” can be used to formulate an agricultural formulation or other composition that includes a purified endophyte preparation. As used herein an “agriculturally compatible carrier” refers to any material, other than water, that can be added to a plant element without causing or having an adverse effect on the plant element (e.g., reducing seed germination) or the plant that grows from the plant element, or the like.

In some embodiments, the formulation can include a tackifier or adherent. Such agents are useful for combining the bacterial population of the invention with carriers that can contain other compounds (e.g., control agents that are not biologic), to yield a coating composition. Such compositions help create coatings around the plant or seed to maintain contact between the microbe and other agents with the plant or plant part. In some embodiments, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amie (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some embodiments the formulation includes, for example, solid carriers such as talc, fullers earth, bentonite, kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran maybe used. The liquid carriers include vegetable oils such as soybean oil and cottonseed oil, glycerol, ethylene glycol, polyethylene glycol, propylene glycol, polypropylene glycol, etc.

In some embodiments, the abundance of an endophyte can be estimated by methods well known in the art including, but not limited to, qPCR, community sequencing, flow cytometry, and/or counting colony-forming units. As used herein, a “colony-forming unit” (“CFU”) is used as a measure of viable microorganisms in a sample. A CFU is an individual viable cell capable of forming on a solid medium a visible colony whose individual cells are derived by cell division from one parental cell.

In some embodiments, the synthetic composition of the present invention comprises one or more of the following: antimicrobial, fungicide, nematicide, bactericide, insecticide, or herbicide.

In some embodiments, a treatment is applied mechanically or manually or artificially inoculated to a plant element in a seed treatment, root wash, seedling soak, foliar application, floral application, soil inoculum, in-furrow application, sidedress application, soil pre-treatment, wound inoculation, drip tape irrigation, vector-mediation via a pollinator, injection, osmopriming, hydroponics, aquaponics, aeroponics, and combinations thereof. Application to the plant may be achieved, for example, as a powder for surface deposition onto plant leaves, as a spray to the whole plant or selected plant element, as part of a drip to the soil or the roots, or as a coating onto the plant element prior to or after planting. Such examples are meant to be illustrative and not limiting to the scope of the invention.

In some embodiments, the invention described herein provides a synthetic composition comprising one or more endophytes capable of improving plant health, wherein the one or more endophytes is a member of the Class Gammaproteobacteria. In some embodiments, the one or more endophytes is a member of the Order Enterobacterales. In some embodiments, the one or more endophytes is a member of the Family Enterobacteriaceae. In some embodiments, the one or more endophytes is a member of the Genus Kosakonia. In some embodiments, the one or more sequences are selected from Table 2A or Table 2B. In some embodiments, the one or more endophytes comprise one or more polynucleotide sequences at least 95%, at least 96%, at least 97%, at least 97%, at least 98%, at least 99%, or 100% identical to one or more of SEQ ID NOs. 1-1089. In some embodiments, the one or more endophytes are capable of producing a protein whose amino acid sequence is at least 95%, at least 96%, at least 97%, at least 97%, at least 98%, at least 99%, or 100% identical to one or more of SEQ ID NOs. 1090-2051.

In some embodiments of any of the synthetic compositions described herein, the endophytes comprise at least 2, 3, 4, 5, 6, 7, 8, 9, 10, 15, 20, 25, 30, 35, 40, 45, 50, 55, 60, 65, 70, 75, 80, or all of the polynucleotide sequences having SEQ ID NOs. 1-1089. In some embodiments of any of the synthetic compositions described herein, the endophytes are capable of producing at least 2, 3, 4, 5, 6, 7, 8, 9, 10, 15, 20, 25, 30, 35, 40, 45, 50, 55, 60, 65, 70, 75, 80, or all of the proteins having amino acid sequences selected from SEQ ID NOs. 1090-2051.

In some embodiments of any of the synthetic compositions described herein, the endophytes comprise at least 2, 3, 4, 5, 6, 7, 8, 9, 10, 15, 20, 25, 30, 35, 40, 45, 50, 55 genes of a tailocin gene cluster, where the genes have 97% identity or greater to one or more polynucleotide sequences SEQ ID NOs. 287-933. In some embodiments of any of the synthetic compositions described herein, the endophytes are capable of producing at least 2, 3, 4, 5, 6, 7, 8, 9, 10, 15, 20, 25, 30, 35, 40, 45, 50, 55 of the tailocin proteins having amino acid sequences selected from SEQ ID NOs. 1318-1922.

In some embodiments of any of the synthetic compositions described herein, the endophytes comprise at least 2, 3, 4, 5, 6, 7, 8, 9 genes of a Type VI secretion system, where the genes have 97% identity or greater to one or more polynucleotide sequences SEQ ID NOs. 934-1014. In some embodiments of any of the synthetic compositions described herein, the endophytes are capable of producing at least 2, 3, 4, 5, 6, 7, 8, 9 of the Type VI secretion system proteins having amino acid sequences selected from SEQ ID NOs. 1923-1981.

In some embodiments of any of the synthetic compositions described herein, the endophytes comprise at least 2, 3, 4, 5, 6, 7, 8, 9 Type VI secretion system putative effector genes, where the genes have 97% identity or greater to one or more polynucleotide sequences SEQ ID NOs. 1015-1089. In some embodiments of any of the synthetic compositions described herein, the endophytes are capable of producing at least 2, 3, 4, 5, 6, 7, 8, 9 Type VI secretion system putative effector proteins having amino acid sequences selected from SEQ ID NOs. 1982-2051.

In some embodiments of any of the synthetic compositions described herein, the endophytes comprise at least 2, 3, 4, 5, 6, 7, 8, 9 flagellin genes, where the genes have 97% identity or greater to one or more polynucleotide sequences SEQ ID NOs. 1-239. In some embodiments of any of the synthetic compositions described herein, the endophytes are capable of producing at least 2, 3, 4, 5, 6, 7, 8, 9 of the flagellin proteins having amino acid sequences selected from SEQ ID NOs. 1090-1272.

In some embodiments of any of the synthetic compositions described herein, the endophytes comprise at least 2, 3, 4, 5, 6, 7, 8, 9 O-Antigen biosynthesis genes, where the genes have 97% identity or greater to one or more polynucleotide sequences SEQ ID NOs. 240-253. In some embodiments of any of the synthetic compositions described herein, the endophytes are capable of producing at least 2, 3, 4, 5, 6, 7, 8, 9 of the O-Antigen biosynthesis proteins having amino acid sequences selected from SEQ ID NOs. 1273-1285.

In some embodiments of any of the synthetic compositions described herein, the endophytes comprise at least 2, 3, 4, 5, 6, 7, 8, 9 pseudaminic acid biosynthesis genes, where the genes have 97% identity or greater to one or more polynucleotide sequences SEQ ID NOs. 254-286. In some embodiments of any of the synthetic compositions described herein, the endophytes are capable of producing at least 2, 3, 4, 5, 6, 7, 8, 9 of the pseudaminic acid biosynthesis proteins having amino acid sequences selected from SEQ ID NOs. 1286-1317.

In some embodiments of any of the synthetic compositions described herein, the endophytes comprise 1) at least 1, 2, 3, 4, 5, 6, 7, 8, 9, 10, 15, 20, 25, 30, 35, 40, 45, 50, 55 genes of a tailocin gene cluster where the genes have 97% identity or greater to one or more polynucleotide sequences SEQ ID NOs. 287-933, 2) at least 1, 2, 3, 4, 5, 6, 7, 8, 9 genes of a Type VI secretion system where the genes have 97% identity or greater to one or more polynucleotide sequences SEQ ID NOs. 934-1014, 3) at least 1, 2, 3, 4, 5, 6, 7, 8, 9 flagellin genes where the genes have 97% identity or greater to one or more polynucleotide sequences SEQ ID NOs. 1-239, 4) at least 1, 2, 3, 4, 5, 6, 7, 8, 9 O-Antigen biosynthesis genes, where the genes have 97% identity or greater to one or more polynucleotide sequences SEQ ID NOs. 240-253, 5) at least 1, 2, 3, 4, 5, 6, 7, 8, 9 pseudaminic acid biosynthesis genes, where the genes have 97% identity or greater to one or more polynucleotide sequences SEQ ID NOs. 254-286, and 6). at least 1, 2, 3, 4, 5, 6, 7, 8, 9 Type VI secretion system putative effector genes where the genes have 97% identity or greater to one or more polynucleotide sequences SEQ ID NOs. 1015-1089.

In some embodiments of any of the synthetic compositions described herein, the synthetic compositions comprise at least 2, 3, 4, 5, 6, 7, 8, 9, 10, 15 or 20 or more endophytes. In some embodiments, the one or more endophytes comprise at least 2, 3, 4, 5, 6, 7, 8, 9, 10, 15 or 20 or more endophytes. In some embodiments, the one or more endophytes are distinct individual organisms or distinct members of different genetic derivation or taxa.

### Methods for Improving Plant Health

In some embodiments, the invention provides methods of improving plant health comprising heterologously disposing one or more endophytes to a plant element in an effective amount to increase a trait of agronomic importance in the plant derived from the treated plant element relative to a plant derived from a reference plant element. In some embodiments, the one or more endophytes are a component of a treatment formulation. In some embodiments, the one or more endophytes are a component of a synthetic composition.

In some embodiments, the invention provides methods of improving plant health comprising creating any of the synthetic compositions described herein, wherein the synthetic composition comprises any of the plant elements of any of the plants described herein and any of the one or more endophytes described herein. In some embodiments, the synthetic composition comprises any of the treatment formulations described herein and any of the one or more endophytes described herein. In some embodiments, the synthetic composition additionally comprises a growth medium or growth environment. A growth environment is a natural or artificially constructed surrounding capable of supporting life of a plant. In some embodiments, the growth medium is soil. In some embodiments, the growth medium is a culture fluid suitable for propagation of an endophyte or plant tissue culture. In some embodiments, the method comprises a step of applying the synthetic composition to a growth medium. In some embodiments, the synthetic composition is applied before one or more plant elements are placed in or on the growth medium. In some embodiments, the synthetic composition is applied after one or more plant elements are placed in or on the growth medium. In some embodiments, the method comprises a step of germinating the plants. In some embodiments, the method comprises a step of growing the plants. For example, the plants may be grown in the plant vigor assays, greenhouse assessments, or field trials described herein. In some embodiments, the method comprises a step of growing the plants to maturity. In some embodiments, where the plants are commercially produced, maturity is the stage at which the plant is normally harvested.

In some embodiments of any of the methods described herein, plant health may be improved for plants in a stress condition. In some embodiments, the stress condition is a biotic or abiotic stress, or a combination of one or more biotic or abiotic stresses. In some embodiments of any of the methods described herein, the stress condition is an abiotic stress selected from the group consisting of: drought stress, salt stress, metal stress, heat stress, cold stress, low nutrient stress (alternately referred to herein as nutrient deficiency or growth in nutrient deficient conditions), and excess water stress, and combinations thereof. In some embodiments of any of the methods described herein, the stress condition is a biotic stress selected from the group consisting of: insect infestation, nematode infestation, complex infection, fungal infection, bacterial infection, oomycete infection, protozoal infection, viral infection, herbivore grazing, and combinations thereof. Stress tolerance is exemplified by improvement of one or more other traits of agronomic importance when compared with a reference plant, reference plant element, or reference population. For example, biotic stress tolerance may be shown by decreased pathogen load of tissues, decreased area of chlorotic tissue, decreased necrosis, improved growth, increased survival, increased biomass, increased shoot height, increased root length, etc. relative to a reference.

### EXAMPLES

### Example 1. Isolation and Identification of Endophytes

Endophytes of the present invention were isolated from the sources listed in Table 1.

TABLE 1
Sources of microbes of the present invention

| | | Isolation |
|---|---|---|
| MIC-ID | Isolated From | Tissue |
| MIC-70076 | Zea mays L. subsp. mays Kokoma | Surface |
| | (landrace maize), obtained from | sterilized |
| | USDA North Central Regional PI | seeds |
| | Station, PI 213733 | |
| MIC-24837 | Glycine max (Soybeans) in flood | |
| | conditions | |
| MIC-61954 | Gossypium hirsutum (Cotton) in | |
| | disease-stress conditions | |
| MIC-81265 | Zea mays (Corn) in temperate | |
| | climate, cold-stress conditions | |
| MIC-73019 | Modern commercial Zea mays | Surface |
| | (Corn) | sterilized |
| | | seeds |
| MIC-52924 | Glycine max (Soybeans) in | |
| | temperate climate, flood conditions | |
| MIC-94458 | Avena sativa (Oats) from | |
| | temperate climate | |
| MIC-46385 | Avena sativa (Oats) from | |
| | temperate climate | |
| MIC-62164 | Avena sativa (Oats) from | |
| | temperate climate | |
| MIC-30352 | Avena sativa (Oats) from | |
| | temperate climate | |
| MIC-82867 | Avena sativa (Oats) from | |
| | temperate climate | |
| MIC-84492 | Glycine max (Soybeans) in | |
| | temperate climate, cold-stress conditions | |
| MIC-85267 | Glycine max (Soybeans) in | |
| | temperate climate, low nutrient conditions | |
| MIC-50391 | Glycine max (Soybeans) in | |
| | temperate climate, nematode-stress conditions | |
| MIC-69701 | Glycine max (Soybeans) in | |
| | temperate climate, nematode-stress conditions | |
| MIC-19814 | Glycine max (Soybeans) from | Seed- |
| | temperate climate | associated |
| MIC-55579 | Glycine max (Soybeans) in | |
| | flood conditions | |
| MIC-80455 | Gossypium hirsutum (Cotton) | |
| MIC-87588 | Gossypium hirsutum (Cotton) | |
| MIC-86605 | Gossypium hirsutum (Cotton) | |
| MIC-54642 | Gossypium hirsutum (Cotton) | |
| MIC-29662 | Gossypium hirsutum (Cotton) in | |
| | disease-stress conditions | |
| MIC-87198 | Gossypium hirsutum (Cotton) in | |
| | drought conditions | |
| MIC-36254 | Gossypium hirsutum (Cotton) in | |
| | temperate climate, low nutrient conditions | |
| MIC-73547 | Gossypium hirsutum (Cotton) in | |
| | temperate climate, low nutrient conditions | |
| MIC-94504 | Gossypium hirsutum (Cotton) in | |
| | drought conditions | |
| MIC-68773 | Malva parvifolia (cheeseweed) | |
| | from temperate climate | |
| MIC-83740 | Malva parvifolia (cheeseweed) | |
| | from temperate climate | |
| MIC-54778 | Phaseolus vulgaris (Kidney bean) | Seed- |
| | from temperate climate | associated |
| MIC-14970 | Sorghum bicolor (Sorghum) from | |
| | temperate climate | |
| MIC-11290 | Sorghum bicolor (Sorghum) from | |
| | temperate climate | |
| MIC-19845 | Zea mays (Corn) in temperate | |
| | climate, cold-stress conditions | |
| MIC-88834 | Zea mays (Corn) in temperate | |
| | climate, cold-stress conditions | |
| MIC-87084 | Zea mays (Corn) in temperate | |
| | climate, insect-stress conditions | |
| MIC-36497 | Zea mays (Corn) in temperate | |
| | climate, insect-stress conditions | |
| MIC-90405 | Zea mays (Corn) in temperate | |
| | climate, low nutrient conditions | |
| MIC-75437 | Zea mays (Corn) in temperate | |
| | climate, low nutrient conditions | |
| MIC-14439 | Zea mays (Corn) in temperate | |
| | climate, low nutrient conditions | |
| MIC-38993 | Zea mays (Corn) in temperate | |
| | climate, low nutrient conditions | |
| MIC-20446 | Zea mays (Corn) in temperate | |
| | climate, low nutrient conditions | |
| MIC-94135 | Zea mays (Corn) in temperate | |
| | climate, low nutrient conditions | |
| MIC-29285 | Zea mays (Corn) in temperate | |
| | climate, flood conditions | |
| MIC-82689 | Zea mays (Corn) in temperate | |
| | climate, flood conditions | |
| MIC-83010 | Zea mays L. subsp. mays | Seed |
| | P39 Goodman-Buckler (modern maize), | surface |
| | obtained from USDA North Central | |
| | Regional PI Station, Ames 28186 PI 690333 | |
| MIC-79613 | Zea mays L. subsp. parviglumis | Surface |
| | (Teosinte), obtained from USDA North | sterilized |
| | Central Regional PI Station, PI 384062 | seeds |
| MIC-53518 | Zea mays L. subsp. parviglumis | Surface |
| | (Teosinte), obtained from USDA North | sterilized |
| | Central Regional PI Station, PI 384062 | seeds |
| MIC-82330 | Wrens Abruzzi Winter Rye, Secale cereale | Surface |
| | | sterilized |
| | | seeds |
| MIC-68901 | Glycine max (Soybeans) from | Seed- |
| | temperate climate | associated |
| MIC-87894 | Glycine max (Soybeans) in | |
| | temperate climate, flood conditions | |

Each sample was processed independently. Each sample was washed in a dilute water and detergent solution; tissue was collected from plants. Samples were surface sterilized by successive rinses: 2 minutes in 10% bleach solution, 2 minutes in 70% ethanol solution, and a rinse with sterile water. The series of rinses was repeated 3 times. The plant tissue was cut into small pieces with sterile scissors and blended with 3, 7 mm steel beads in 5-7.5 ml phosphate buffered solution (PBS). DNA was extracted from the ground tissues using the Magbind Plant DNA kit (Omega, Norcross, Georgia, USA) according to the manufacturer's instructions.

The endophytes were characterized by whole genome sequencing.

Phylogenetic and genomic analyses for bacterial strains. According to the manufacturer's protocol, DNA was extracted from pure cultures using the Omega Mag-Bind Universal Pathogen Kit with a final elution volume of 60 μl (Omega Biotek Inc., Norcross, GA). DNA samples were quantified using a Qubit fluorometer (ThermoFisher Scientific, Waltham, MA) and normalized to 100 ng. DNA was prepared using the Nextera DNA Flex Library Prep Kit according to the manufacturer's instructions (Illumina Inc., San Diego, CA). DNA libraries were quantified via qPCR using the KAPA Library Quantification kit (Roche Sequencing and Life Science, Wilmington, MA) and combined in equimolar concentrations into one 24-sample pool. Libraries were sequenced on a MiSeq using pair-end reads (2×200 bp). Reads were trimmed of adapters and low-quality bases using Cutadapt (version 1.9.1) and assembled into contigs using MEGAHIT (version 1.1.2) (Li, D., Liu, C.-M., Luo, R., Sadakane, K., and Lam, T.-W. 2015. MEGAHIT: an ultra-fast single-node solution for large and complex metagenomics assembly via succinct de Bruijn graph. Bioinformatics. 31:1674-1676). Reads were mapped to contigs using Bowtie2 (version 2.3.4) (Langmead, B., and Salzberg, S. L. 2012. Fast gapped-read alignment with bowtie 2. Nat Methods. 9 Available at: doi.org/10.1038/nmeth.1923), and contigs were assembled into scaffolds using BESST (2.2.8) (Sahlin, K., Vezzi, F., Nystedt, B., Lundeberg, J., and Arvestad, L. 2014. BESST-efficient scaffolding of large fragmented assemblies. BMC bioinformatics. 15:281).

Genes for phylogenetic analyses were extracted from genome assemblies using barrnap (Seemann, T. 2019. barrnap 0.9: rapid ribosomal RNA prediction. Available at: github.com/tseemann/barrnap) or blast (Altschul, S. F., Madden, T. L., Schäffer, A. A., Zhang, J., Zhang, Z., Miller, W., et al. 1997. Gapped BLAST and PSI-BLAST: A new generation of protein database search programs. Nucleic Acids Research. 25:3389-3402). Homologous DNA sequences from types or other, likely correctly identified strains were retrieved from GenBank and aligned using MAFFT (Katoh, K., and Standley, D. M. 2013. MAFFT multiple sequence alignment software version 7: improvements in performance and usability. Molecular Biology and Evolution. 30:772-780), or other software. Single or multilocus phylogenetic analyses were performed using PAUP (Swofford, D. L. 2002. PAUP: Phylogenetic Analysis Using Parsimony (and Other Methods). Version 4. Sunderland, Massachusetts: Sinauer Associates) or similar software.

16S rRNA gene sequences were extracted from genome assemblies using barrnap (Seemann 2019). Phylogenomic analyses were performed using GToTree (Lee, M. D. 2019. Applications and considerations of GToTree: a user-friendly workflow for phylogenomics. Evolutionary Bioinformatics. 15:1176934319862245) with default settings. Average nucleotide identity analyses were performed using the pyani ANIm algorithm (Richter, M., and Rosselló-Móra, R. 2009. Shifting the genomic gold standard for the prokaryotic species definition. Proceedings of the National Academy of Sciences. 106:19126-19131) implemented in the MUMmer package (Kurtz, S., Phillippy, A., Delcher, A. L., Smoot, M., Shumway, M., Antonescu, C., et al. 2004. Versatile and open software for comparing large genomes. Genome biology. 5: R12) retrieved from github.com/widdowquinn/pyani.

Identification of bacterial strains. Bacteria are identified at the species level, if its average nucleotide identity (ANI) was >95% to the genome of a single species represented by its type strain downloaded from GenBank. Phylogenomic analyses were also performed if a bacteria had >1 species with >95% ANI, or the gap between the top two ANI hits was <3%, in this case, the bacteria is identified at the genus and species if it had a single sister group with >70% bootstrap support.

All bacteria of the present invention were identified as Kingdom: Bacteria, Phylum: Proteobacteria, Class: Gammaproteobacteria, Order: Enterobacterales, Family: Enterobacteriaceae, Genus: Kosakonia, Species: cowanii.

MIC-70076 was deposited with as Deposit ID.

TABLE 2A
Polynucleotide sequences of endophytes

| SEQ ID | Sequence Description | Endophytes |
|---|---|---|
| 1 | Flagellin | MIC-70076 |
| 2 | Flagellin | MIC-70076 |
| 3 | Flagellin | MIC-70076 |
| 4 | Flagellin | MIC-70076 |
| 5 | Flagellin | MIC-70076 |
| 6 | Flagellin | MIC-70076 |
| 7 | Flagellin | MIC-70076 |
| 8 | Flagellin | MIC-70076 |
| 9 | Flagellin | MIC-80455, MIC-87588, MIC-86605, |
| | | MIC-54642, MIC-24837, MIC-55579, |
| | | MIC-61954, MIC-29662, MIC-38993, |
| | | MIC-14970, MIC-11290, MIC-82689 |
| 10 | Flagellin | MIC-68901, MIC-81265, MIC-88834, |
| | | MIC-46385 |
| 11 | Flagellin | MIC-68773, MIC-86605, MIC-54642, |
| | | MIC-61954, MIC-29662, MIC-11290 |
| 12 | Flagellin | MIC-87588, MIC-86605, MIC-54642, |
| | | MIC-61954, MIC-29662 |
| 13 | Flagellin | MIC-87588, MIC-86605, MIC-54642, |
| | | MIC-61954, MIC-29662 |
| 14 | Flagellin | MIC-70076, MIC-73019, MIC-73547 |
| 15 | Flagellin | MIC-70076, MIC-73019, MIC-73547 |
| 16 | Flagellin | MIC-70076, MIC-73019, MIC-73547 |
| 17 | Flagellin | MIC-68773, MIC-86605, MIC-61954, |
| | | MIC-29662, MIC-11290 |
| 18 | Flagellin | MIC-87588, MIC-86605, MIC-61954, |
| | | MIC-29662, MIC-14970, MIC-11290 |
| 19 | Flagellin | MIC-81265 |
| 20 | Flagellin | MIC-61954 |
| 21 | Flagellin | MIC-61954 |
| 22 | Flagellin | MIC-61954 |
| 23 | Flagellin | MIC-61954 |
| 24 | Flagellin | MIC-68901 |
| 25 | Flagellin | MIC-68901 |
| 26 | Flagellin | MIC-68901 |
| 27 | Flagellin | MIC-68901 |
| 28 | Flagellin | MIC-52924 |
| 29 | Flagellin | MIC-73019, MIC-73547 |
| 30 | Flagellin | MIC-73019, MIC-73547 |
| 31 | Flagellin | MIC-73019, MIC-73547 |
| 32 | Flagellin | MIC-73019, MIC-73547 |
| 33 | Flagellin | MIC-82330 |
| 34 | Flagellin | MIC-82330 |
| 35 | Flagellin | MIC-82330 |
| 36 | Flagellin | MIC-82330 |
| 37 | Flagellin | MIC-82330 |
| 38 | Flagellin | MIC-82330 |
| 39 | Flagellin | MIC-68773 |
| 40 | Flagellin | MIC-68773 |
| 41 | Flagellin | MIC-68773 |
| 42 | Flagellin | MIC-68773 |
| 43 | Flagellin | MIC-68773 |
| 44 | Flagellin | MIC-68773 |
| 45 | Flagellin | MIC-68773 |
| 46 | Flagellin | MIC-54778 |
| 47 | Flagellin | MIC-54778 |
| 48 | Flagellin | MIC-54778 |
| 49 | Flagellin | MIC-54778 |
| 50 | Flagellin | MIC-54778 |
| 51 | Flagellin | MIC-19814 |
| 52 | Flagellin | MIC-19814 |
| 53 | Flagellin | MIC-87588 |
| 54 | Flagellin | MIC-87588 |
| 55 | Flagellin | MIC-24837 |
| 56 | Flagellin | MIC-24837 |
| 57 | Flagellin | MIC-87198 |
| 58 | Flagellin | MIC-87198 |
| 59 | Flagellin | MIC-87198 |
| 60 | Flagellin | MIC-87198 |
| 61 | Flagellin | MIC-87198 |
| 62 | Flagellin | MIC-87198 |
| 63 | Flagellin | MIC-87198 |
| 64 | Flagellin | MIC-90405 |
| 65 | Flagellin | MIC-90405 |
| 66 | Flagellin | MIC-90405 |
| 67 | Flagellin | MIC-90405 |
| 68 | Flagellin | MIC-90405 |
| 69 | Flagellin | MIC-90405 |
| 70 | Flagellin | MIC-90405 |
| 71 | Flagellin | MIC-90405 |
| 72 | Flagellin | MIC-87894 |
| 73 | Flagellin | MIC-14970 |
| 74 | Flagellin | MIC-14970 |
| 75 | Flagellin | MIC-54642 |
| 76 | Flagellin | MIC-54642 |
| 77 | Flagellin | MIC-88834 |
| 78 | Flagellin | MIC-88834 |
| 79 | Flagellin | MIC-88834 |
| 80 | Flagellin | MIC-14439 |
| 81 | Flagellin | MIC-14439 |
| 82 | Flagellin | MIC-14439 |
| 83 | Flagellin | MIC-14439 |
| 84 | Flagellin | MIC-14439 |
| 85 | Flagellin | MIC-14439 |
| 86 | Flagellin | MIC-79613 |
| 87 | Flagellin | MIC-79613 |
| 88 | Flagellin | MIC-79613 |
| 89 | Flagellin | MIC-79613 |
| 90 | Flagellin | MIC-79613, MIC-53518 |
| 91 | Flagellin | MIC-79613, MIC-53518 |
| 92 | Flagellin | MIC-79613, MIC-53518 |
| 93 | Flagellin | MIC-53518 |
| 94 | Flagellin | MIC-53518 |
| 95 | Flagellin | MIC-53518 |
| 96 | Flagellin | MIC-53518 |
| 97 | Flagellin | MIC-82330, MIC-84492 |
| 98 | Flagellin | MIC-82330, MIC-84492 |
| 99 | Flagellin | MIC-83010 |
| 100 | Flagellin | MIC-83010 |
| 101 | Flagellin | MIC-83010 |
| 102 | Flagellin | MIC-83010, MIC-75437 |
| 103 | Flagellin | MIC-83010, MIC-75437, MIC-20446 |
| 104 | Flagellin | MIC-83740 |
| 105 | Flagellin | MIC-83740 |
| 106 | Flagellin | MIC-83740 |
| 107 | Flagellin | MIC-83740 |
| 108 | Flagellin | MIC-83740 |
| 109 | Flagellin | MIC-83740 |
| 110 | Flagellin | MIC-83740 |
| 111 | Flagellin | MIC-83740 |
| 112 | Flagellin | MIC-83740 |
| 113 | Flagellin | MIC-80455 |
| 114 | Flagellin | MIC-80455 |
| 115 | Flagellin | MIC-80455 |
| 116 | Flagellin | MIC-80455 |
| 117 | Flagellin | MIC-80455 |
| 118 | Flagellin | MIC-80455 |
| 119 | Flagellin | MIC-86605 |
| 120 | Flagellin | MIC-86605 |
| 121 | Flagellin | MIC-86605 |
| 122 | Flagellin | MIC-54642, MIC-11290 |
| 123 | Flagellin | MIC-94504 |
| 124 | Flagellin | MIC-94504 |
| 125 | Flagellin | MIC-94504 |
| 126 | Flagellin | MIC-94504 |
| 127 | Flagellin | MIC-94504 |
| 128 | Flagellin | MIC-94504 |
| 129 | Flagellin | MIC-94504 |
| 130 | Flagellin | MIC-24837, MIC-55579 |
| 131 | Flagellin | MIC-24837, MIC-55579 |
| 132 | Flagellin | MIC-24837, MIC-55579 |
| 133 | Flagellin | MIC-24837, MIC-55579 |
| 134 | Flagellin | MIC-55579 |
| 135 | Flagellin | MIC-55579 |
| 136 | Flagellin | MIC-55579 |
| 137 | Flagellin | MIC-55579 |
| 138 | Flagellin | MIC-29662, MIC-11290 |
| 139 | Flagellin | MIC-19845 |
| 140 | Flagellin | MIC-19845 |
| 141 | Flagellin | MIC-19845 |
| 142 | Flagellin | MIC-19845 |
| 143 | Flagellin | MIC-19845 |
| 144 | Flagellin | MIC-19845 |
| 145 | Flagellin | MIC-19845 |
| 146 | Flagellin | MIC-84492 |
| 147 | Flagellin | MIC-84492 |
| 148 | Flagellin | MIC-84492 |
| 149 | Flagellin | MIC-84492 |
| 150 | Flagellin | MIC-84492 |
| 151 | Flagellin | MIC-84492 |
| 152 | Flagellin | MIC-84492 |
| 153 | Flagellin | MIC-50391, MIC-69701, MIC-52924 |
| 154 | Flagellin | MIC-50391, MIC-69701, MIC-52924 |
| 155 | Flagellin | MIC-50391, MIC-69701, MIC-52924 |
| 156 | Flagellin | MIC-50391, MIC-69701, MIC-52924 |
| 157 | Flagellin | MIC-85267 |
| 158 | Flagellin | MIC-85267 |
| 159 | Flagellin | MIC-85267 |
| 160 | Flagellin | MIC-85267 |
| 161 | Flagellin | MIC-85267 |
| 162 | Flagellin | MIC-85267 |
| 163 | Flagellin | MIC-85267 |
| 164 | Flagellin | MIC-85267 |
| 165 | Flagellin | MIC-90405, MIC-38993 |
| 166 | Flagellin | MIC-75437 |
| 167 | Flagellin | MIC-75437 |
| 168 | Flagellin | MIC-75437, MIC-20446 |
| 169 | Flagellin | MIC-75437, MIC-20446 |
| 170 | Flagellin | MIC-38993 |
| 171 | Flagellin | MIC-38993 |
| 172 | Flagellin | MIC-38993 |
| 173 | Flagellin | MIC-38993 |
| 174 | Flagellin | MIC-38993 |
| 175 | Flagellin | MIC-20446 |
| 176 | Flagellin | MIC-20446 |
| 177 | Flagellin | MIC-94135 |
| 178 | Flagellin | MIC-94135 |
| 179 | Flagellin | MIC-94135 |
| 180 | Flagellin | MIC-94458 |
| 181 | Flagellin | MIC-94458 |
| 182 | Flagellin | MIC-94458 |
| 183 | Flagellin | MIC-94458, MIC-30352 |
| 184 | Flagellin | MIC-94458, MIC-30352, MIC-82867 |
| 185 | Flagellin | MIC-46385, MIC-62164, MIC-87894 |
| 186 | Flagellin | MIC-46385, MIC-87894 |
| 187 | Flagellin | MIC-46385, MIC-87894 |
| 188 | Flagellin | MIC-46385, MIC-87894 |
| 189 | Flagellin | MIC-62164 |
| 190 | Flagellin | MIC-62164 |
| 191 | Flagellin | MIC-30352 |
| 192 | Flagellin | MIC-82867 |
| 193 | Flagellin | MIC-82867 |
| 194 | Flagellin | MIC-82867 |
| 195 | Flagellin | MIC-82867 |
| 196 | Flagellin | MIC-36254 |
| 197 | Flagellin | MIC-36254 |
| 198 | Flagellin | MIC-36254 |
| 199 | Flagellin | MIC-36254 |
| 200 | Flagellin | MIC-36254 |
| 201 | Flagellin | MIC-36254 |
| 202 | Flagellin | MIC-36254 |
| 203 | Flagellin | MIC-36254 |
| 204 | Flagellin | MIC-36254 |
| 205 | Flagellin | MIC-36254 |
| 206 | Flagellin | MIC-73547 |
| 207 | Flagellin | MIC-73547 |
| 208 | Flagellin | MIC-73547 |
| 209 | Flagellin | MIC-14970, MIC-11290 |
| 210 | Flagellin | MIC-14970, MIC-11290 |
| 211 | Flagellin | MIC-87084 |
| 212 | Flagellin | MIC-87084 |
| 213 | Flagellin | MIC-87084 |
| 214 | Flagellin | MIC-87084 |
| 215 | Flagellin | MIC-87084 |
| 216 | Flagellin | MIC-87084 |
| 217 | Flagellin | MIC-36497 |
| 218 | Flagellin | MIC-36497 |
| 219 | Flagellin | MIC-36497 |
| 220 | Flagellin | MIC-36497 |
| 221 | Flagellin | MIC-36497 |
| 222 | Flagellin | MIC-36497 |
| 223 | Flagellin | MIC-36497 |
| 224 | Flagellin | MIC-36497 |
| 225 | Flagellin | MIC-29285 |
| 226 | Flagellin | MIC-29285 |
| 227 | Flagellin | MIC-29285 |
| 228 | Flagellin | MIC-29285 |
| 229 | Flagellin | MIC-29285 |
| 230 | Flagellin | MIC-29285 |
| 231 | Flagellin | MIC-29285 |
| 232 | Flagellin | MIC-82689 |
| 233 | Flagellin | MIC-82689 |
| 234 | Flagellin | MIC-82689 |
| 235 | Flagellin | MIC-82689 |
| 236 | Flagellin | MIC-82689 |
| 237 | Flagellin | MIC-82689 |
| 238 | Flagellin | MIC-82689 |
| 239 | Flagellin | MIC-82689 |
| 240 | O-Antigen | MIC-70076, MIC-73019, MIC-73547 |
| | biosynthesis | |
| 241 | O-Antigen | MIC-70076, MIC-73019, MIC-73547 |
| | biosynthesis | |
| 242 | O-Antigen | MIC-70076, MIC-73019, MIC-73547 |
| | biosynthesis | |
| 243 | O-Antigen | MIC-70076, MIC-73019, MIC-73547 |
| | biosynthesis | |
| 244 | O-Antigen | MIC-19845 |
| | biosynthesis | |
| 245 | O-Antigen | MIC-85267 |
| | biosynthesis | |
| 246 | O-Antigen | MIC-38993 |
| | biosynthesis | |
| 247 | O-Antigen | MIC-38993 |
| | biosynthesis | |
| 248 | O-Antigen | MIC-38993 |
| | biosynthesis | |
| 249 | O-Antigen | MIC-38993, MIC-82689 |
| | biosynthesis | |
| 250 | O-Antigen | MIC-36497 |
| | biosynthesis | |
| 251 | O-Antigen | MIC-82689 |
| | biosynthesis | |
| 252 | O-Antigen | MIC-82689 |
| | biosynthesis | |
| 253 | O-Antigen | MIC-82689 |
| | biosynthesis | |
| 254 | pseudaminic | MIC-80455, MIC-87588, MIC-86605, |
| | acid biosynthesis | MIC-54642, MIC-24837, MIC-55579, |
| | | MIC-61954, MIC-29662, MIC-90405, |
| | | MIC-14970, MIC-11290 |
| 255 | pseudaminic | MIC-87588, MIC-86605, MIC-54642, |
| | acid biosynthesis | MIC-24837, MIC-55579, MIC-61954, |
| | | MIC-29662, MIC-90405, MIC-14970, |
| | | MIC-11290 |
| 256 | pseudaminic | MIC-70076, MIC-73019, MIC-73547 |
| | acid biosynthesis | |
| 257 | pseudaminic | MIC-70076, MIC-73019, MIC-73547 |
| | acid biosynthesis | |
| 258 | pseudaminic | MIC-70076, MIC-73019, MIC-73547 |
| | acid biosynthesis | |
| 259 | pseudaminic | MIC-70076, MIC-73019, MIC-73547 |
| | acid biosynthesis | |
| 260 | pseudaminic | MIC-70076, MIC-73019, MIC-73547 |
| | acid biosynthesis | |
| 261 | pseudaminic | MIC-70076, MIC-73019, MIC-73547 |
| | acid biosynthesis | |
| 262 | pseudaminic | MIC-70076, MIC-73019, MIC-73547 |
| | acid biosynthesis | |
| 263 | pseudaminic | MIC-79613, MIC-53518 |
| | acid biosynthesis | |
| 264 | pseudaminic | MIC-79613, MIC-53518 |
| | acid biosynthesis | |
| 265 | pseudaminic | MIC-80455 |
| | acid biosynthesis | |
| 266 | pseudaminic | MIC-19845 |
| | acid biosynthesis | |
| 267 | pseudaminic | MIC-19845 |
| | acid biosynthesis | |
| 268 | pseudaminic | MIC-19845 |
| | acid biosynthesis | |
| 269 | pseudaminic | MIC-19845 |
| | acid biosynthesis | |
| 270 | pseudaminic | MIC-50391, MIC-69701, MIC-52924 |
| | acid biosynthesis | |
| 271 | pseudaminic | MIC-85267 |
| | acid biosynthesis | |
| 272 | pseudaminic | MIC-38993 |
| | acid biosynthesis | |
| 273 | pseudaminic | MIC-38993 |
| | acid biosynthesis | |
| 274 | pseudaminic | MIC-38993 |
| | acid biosynthesis | |
| 275 | pseudaminic | MIC-38993 |
| | acid biosynthesis | |
| 276 | pseudaminic | MIC-38993 |
| | acid biosynthesis | |
| 277 | pseudaminic | MIC-38993 |
| | acid biosynthesis | |
| 278 | pseudaminic | MIC-38993 |
| | acid biosynthesis | |
| 279 | pseudaminic | MIC-94458, MIC-30352, MIC-82867 |
| | acid biosynthesis | |
| 280 | pseudaminic | MIC-82689 |
| | acid biosynthesis | |
| 281 | pseudaminic | MIC-82689 |
| | acid biosynthesis | |
| 282 | pseudaminic | MIC-82689 |
| | acid biosynthesis | |
| 283 | pseudaminic | MIC-82689 |
| | acid biosynthesis | |
| 284 | pseudaminic | MIC-82689 |
| | acid biosynthesis | |
| 285 | pseudaminic | MIC-82689 |
| | acid biosynthesis | |
| 286 | pseudaminic | MIC-82689 |
| | acid biosynthesis | |
| 287 | Tailocin | MIC-87588, MIC-86605, MIC-54642, |
| | gene cluster | MIC-61954, MIC-29662 |
| 288 | Tailocin | MIC-70076, MIC-73019, MIC-24837, |
| | gene cluster | MIC-55579, MIC-73547 |
| 289 | Tailocin | MIC-70076, MIC-73019, MIC-24837, |
| | gene cluster | MIC-55579, MIC-73547 |
| 290 | Tailocin | MIC-70076, MIC-73019, MIC-73547 |
| | gene cluster | |
| 291 | Tailocin | MIC-70076, MIC-73019, MIC-73547 |
| | gene cluster | |
| 292 | Tailocin | MIC-70076, MIC-73019, MIC-38993, |
| | gene cluster | MIC-73547 |
| 293 | Tailocin | MIC-70076, MIC-73019, MIC-38993, |
| | gene cluster | MIC-73547 |
| 294 | Tailocin | MIC-70076, MIC-73019, MIC-73547 |
| | gene cluster | |
| 295 | Tailocin | MIC-70076, MIC-73019, MIC-73547 |
| | gene cluster | |
| 296 | Tailocin | MIC-70076, MIC-73019, MIC-73547 |
| | gene cluster | |
| 297 | Tailocin | MIC-70076, MIC-73019, MIC-73547 |
| | gene cluster | |
| 298 | Tailocin | MIC-70076, MIC-73019, MIC-73547 |
| | gene cluster | |
| 299 | Tailocin | MIC-70076, MIC-73019, MIC-73547 |
| | gene cluster | |
| 300 | Tailocin | MIC-70076, MIC-73019, MIC-73547 |
| | gene cluster | |
| 301 | Tailocin | MIC-70076, MIC-73019, MIC-73547 |
| | gene cluster | |
| 302 | Tailocin | MIC-70076, MIC-73019, MIC-73547 |
| | gene cluster | |
| 303 | Tailocin | MIC-70076, MIC-73019, MIC-73547 |
| | gene cluster | |
| 304 | Tailocin | MIC-70076, MIC-73019, MIC-73547 |
| | gene cluster | |
| 305 | Tailocin | MIC-70076, MIC-73019, MIC-73547 |
| | gene cluster | |
| 306 | Tailocin | MIC-70076, MIC-73019, MIC-73547 |
| | gene cluster | |
| 307 | Tailocin | MIC-70076, MIC-73019, MIC-73547 |
| | gene cluster | |
| 308 | Tailocin | MIC-70076, MIC-73019, MIC-73547 |
| | gene cluster | |
| 309 | Tailocin | MIC-70076, MIC-73019, MIC-73547 |
| | gene cluster | |
| 310 | Tailocin | MIC-70076, MIC-73019, MIC-73547 |
| | gene cluster | |
| 311 | Tailocin | MIC-70076, MIC-73019, MIC-38993, |
| | gene cluster | MIC-73547 |
| 312 | Tailocin | MIC-70076, MIC-73019, MIC-73547 |
| | gene cluster | |
| 313 | Tailocin | MIC-70076, MIC-73019, MIC-73547 |
| | gene cluster | |
| 314 | Tailocin | MIC-70076, MIC-73019, MIC-73547 |
| | gene cluster | |
| 315 | Tailocin | MIC-70076, MIC-73019, MIC-73547 |
| | gene cluster | |
| 316 | Tailocin | MIC-70076, MIC-73019, MIC-73547 |
| | gene cluster | |
| 317 | Tailocin | MIC-70076, MIC-73019, MIC-73547 |
| | gene cluster | |
| 318 | Tailocin | MIC-70076, MIC-73019, MIC-73547 |
| | gene cluster | |
| 319 | Tailocin | MIC-70076, MIC-73019, MIC-73547 |
| | gene cluster | |
| 320 | Tailocin | MIC-70076, MIC-73019, MIC-73547 |
| | gene cluster | |
| 321 | Tailocin | MIC-70076, MIC-73019, MIC-73547 |
| | gene cluster | |
| 322 | Tailocin | MIC-70076, MIC-73019, MIC-73547 |
| | gene cluster | |
| 323 | Tailocin | MIC-70076, MIC-73019, MIC-73547 |
| | gene cluster | |
| 324 | Tailocin | MIC-70076, MIC-73019, MIC-73547 |
| | gene cluster | |
| 325 | Tailocin | MIC-70076, MIC-73019, MIC-73547 |
| | gene cluster | |
| 326 | Tailocin | MIC-70076, MIC-73019, MIC-73547 |
| | gene cluster | |
| 327 | Tailocin | MIC-70076, MIC-73019, MIC-73547 |
| | gene cluster | |
| 328 | Tailocin | MIC-70076, MIC-73019, MIC-73547 |
| | gene cluster | |
| 329 | Tailocin | MIC-70076, MIC-73019, MIC-73547 |
| | gene cluster | |
| 330 | Tailocin | MIC-70076, MIC-73019, MIC-73547 |
| | gene cluster | |
| 331 | Tailocin | MIC-70076, MIC-73019, MIC-73547 |
| | gene cluster | |
| 332 | Tailocin | MIC-70076, MIC-73019, MIC-73547 |
| | gene cluster | |
| 333 | Tailocin | MIC-70076, MIC-73019, MIC-73547 |
| | gene cluster | |
| 334 | Tailocin | MIC-70076, MIC-73019, MIC-73547 |
| | gene cluster | |
| 335 | Tailocin | MIC-70076, MIC-73019, MIC-73547 |
| | gene cluster | |
| 336 | Tailocin | MIC-70076, MIC-73019, MIC-73547 |
| | gene cluster | |
| 337 | Tailocin | MIC-70076, MIC-73019, MIC-73547 |
| | gene cluster | |
| 338 | Tailocin | MIC-70076, MIC-73019, MIC-73547 |
| | gene cluster | |
| 339 | Tailocin | MIC-70076, MIC-73019, MIC-73547 |
| | gene cluster | |
| 340 | Tailocin | MIC-70076, MIC-73019, MIC-73547 |
| | gene cluster | |
| 341 | Tailocin | MIC-70076, MIC-73019, MIC-73547 |
| | gene cluster | |
| 342 | Tailocin | MIC-70076, MIC-73019, MIC-73547 |
| | gene cluster | |
| 343 | Tailocin | MIC-82330, MIC-61954, MIC-29662, |
| | gene cluster | MIC-36254 |
| 344 | Tailocin | MIC-68901, MIC-19814 |
| | gene cluster | |
| 345 | Tailocin | MIC-68901, MIC-19814 |
| | gene cluster | |
| 346 | Tailocin | MIC-68901, MIC-19814 |
| | gene cluster | |
| 347 | Tailocin | MIC-68901, MIC-19814 |
| | gene cluster | |
| 348 | Tailocin | MIC-68901, MIC-19814 |
| | gene cluster | |
| 349 | Tailocin | MIC-68901, MIC-19814 |
| | gene cluster | |
| 350 | Tailocin | MIC-68901, MIC-19814 |
| | gene cluster | |
| 351 | Tailocin | MIC-68901, MIC-19814 |
| | gene cluster | |
| 352 | Tailocin | MIC-68901, MIC-19814 |
| | gene cluster | |
| 353 | Tailocin | MIC-68901, MIC-19814 |
| | gene cluster | |
| 354 | Tailocin | MIC-68901, MIC-19814 |
| | gene cluster | |
| 355 | Tailocin | MIC-68901, MIC-19814 |
| | gene cluster | |
| 356 | Tailocin | MIC-68901, MIC-19814 |
| | gene cluster | |
| 357 | Tailocin | MIC-68901, MIC-19814 |
| | gene cluster | |
| 358 | Tailocin | MIC-68901, MIC-19814 |
| | gene cluster | |
| 359 | Tailocin | MIC-68901, MIC-19814 |
| | gene cluster | |
| 360 | Tailocin | MIC-68901, MIC-19814 |
| | gene cluster | |
| 361 | Tailocin | MIC-68901, MIC-19814 |
| | gene cluster | |
| 362 | Tailocin | MIC-68901, MIC-19814 |
| | gene cluster | |
| 363 | Tailocin | MIC-68901, MIC-19814 |
| | gene cluster | |
| 364 | Tailocin | MIC-68901, MIC-19814 |
| | gene cluster | |
| 365 | Tailocin | MIC-68901 |
| | gene cluster | |
| 366 | Tailocin | MIC-68901 |
| | gene cluster | |
| 367 | Tailocin | MIC-68901 |
| | gene cluster | |
| 368 | Tailocin | MIC-68901 |
| | gene cluster | |
| 369 | Tailocin | MIC-68901 |
| | gene cluster | |
| 370 | Tailocin | MIC-68901 |
| | gene cluster | |
| 371 | Tailocin | MIC-52924 |
| | gene cluster | |
| 372 | Tailocin | MIC-52924 |
| | gene cluster | |
| 373 | Tailocin | MIC-52924 |
| | gene cluster | |
| 374 | Tailocin | MIC-52924 |
| | gene cluster | |
| 375 | Tailocin | MIC-83010, MIC-86605, MIC-81265, |
| | gene cluster | MIC-88834, MIC-84492, MIC-75437, |
| | | MIC-20446, MIC-94135, MIC-46385, |
| | | MIC-62164, MIC-87084, MIC-36497, |
| | | MIC-82689 |
| 376 | Tailocin | MIC-81265, MIC-88834 |
| | gene cluster | |
| 377 | Tailocin | MIC-81265, MIC-88834, MIC-84492 |
| | gene cluster | |
| 378 | Tailocin | MIC-81265, MIC-88834, MIC-84492 |
| | gene cluster | |
| 379 | Tailocin | MIC-81265, MIC-88834, MIC-84492, |
| | gene cluster | MIC-75437, MIC-20446 |
| 380 | Tailocin | MIC-81265, MIC-88834, MIC-84492, |
| | gene cluster | MIC-75437, MIC-20446 |
| 381 | Tailocin | MIC-81265, MIC-88834, MIC-84492, |
| | gene cluster | MIC-75437, MIC-20446 |
| 382 | Tailocin | MIC-61954, MIC-29662 |
| | gene cluster | |
| 383 | Tailocin | MIC-61954, MIC-29662 |
| | gene cluster | |
| 384 | Tailocin | MIC-61954, MIC-29662 |
| | gene cluster | |
| 385 | Tailocin | MIC-61954, MIC-29662 |
| | gene cluster | |
| 386 | Tailocin | MIC-61954, MIC-29662 |
| | gene cluster | |
| 387 | Tailocin | MIC-61954, MIC-29662 |
| | gene cluster | |
| 388 | Tailocin | MIC-61954, MIC-29662 |
| | gene cluster | |
| 389 | Tailocin | MIC-61954, MIC-29662 |
| | gene cluster | |
| 390 | Tailocin | MIC-61954, MIC-29662 |
| | gene cluster | |
| 391 | Tailocin | MIC-82330 |
| | gene cluster | |
| 392 | Tailocin | MIC-68773 |
| | gene cluster | |
| 393 | Tailocin | MIC-68773 |
| | gene cluster | |
| 394 | Tailocin | MIC-68773 |
| | gene cluster | |
| 395 | Tailocin | MIC-68773 |
| | gene cluster | |
| 396 | Tailocin | MIC-68773 |
| | gene cluster | |
| 397 | Tailocin | MIC-68773 |
| | gene cluster | |
| 398 | Tailocin | MIC-68773 |
| | gene cluster | |
| 399 | Tailocin | MIC-68773 |
| | gene cluster | |
| 400 | Tailocin | MIC-54778 |
| | gene cluster | |
| 401 | Tailocin | MIC-19814 |
| | gene cluster | |
| 402 | Tailocin | MIC-19814 |
| | gene cluster | |
| 403 | Tailocin | MIC-19814 |
| | gene cluster | |
| 404 | Tailocin | MIC-19814 |
| | gene cluster | |
| 405 | Tailocin | MIC-19814 |
| | gene cluster | |
| 406 | Tailocin | MIC-19814 |
| | gene cluster | |
| 407 | Tailocin | MIC-19814 |
| | gene cluster | |
| 408 | Tailocin | MIC-19814 |
| | gene cluster | |
| 409 | Tailocin | MIC-87198 |
| | gene cluster | |
| 410 | Tailocin | MIC-87198 |
| | gene cluster | |
| 411 | Tailocin | MIC-87198 |
| | gene cluster | |
| 412 | Tailocin | MIC-87198 |
| | gene cluster | |
| 413 | Tailocin | MIC-87198 |
| | gene cluster | |
| 414 | Tailocin | MIC-87198 |
| | gene cluster | |
| 415 | Tailocin | MIC-87198 |
| | gene cluster | |
| 416 | Tailocin | MIC-90405 |
| | gene cluster | |
| 417 | Tailocin | MIC-90405 |
| | gene cluster | |
| 418 | Tailocin | MIC-90405 |
| | gene cluster | |
| 419 | Tailocin | MIC-90405 |
| | gene cluster | |
| 420 | Tailocin | MIC-90405 |
| | gene cluster | |
| 421 | Tailocin | MIC-90405 |
| | gene cluster | |
| 422 | Tailocin | MIC-90405 |
| | gene cluster | |
| 423 | Tailocin | MIC-90405 |
| | gene cluster | |
| 424 | Tailocin | MIC-90405 |
| | gene cluster | |
| 425 | Tailocin | MIC-90405 |
| | gene cluster | |
| 426 | Tailocin | MIC-90405 |
| | gene cluster | |
| 427 | Tailocin | MIC-90405 |
| | gene cluster | |
| 428 | Tailocin | MIC-90405 |
| | gene cluster | |
| 429 | Tailocin | MIC-90405 |
| | gene cluster | |
| 430 | Tailocin | MIC-87894 |
| | gene cluster | |
| 431 | Tailocin | MIC-87894 |
| | gene cluster | |
| 432 | Tailocin | MIC-87894 |
| | gene cluster | |
| 433 | Tailocin | MIC-87894 |
| | gene cluster | |
| 434 | Tailocin | MIC-87894 |
| | gene cluster | |
| 435 | Tailocin | MIC-87894 |
| | gene cluster | |
| 436 | Tailocin | MIC-87894 |
| | gene cluster | |
| 437 | Tailocin | MIC-87894 |
| | gene cluster | |
| 438 | Tailocin | MIC-87894 |
| | gene cluster | |
| 439 | Tailocin | MIC-87894 |
| | gene cluster | |
| 440 | Tailocin | MIC-87894 |
| | gene cluster | |
| 441 | Tailocin | MIC-87894 |
| | gene cluster | |
| 442 | Tailocin | MIC-14439 |
| | gene cluster | |
| 443 | Tailocin | MIC-14439 |
| | gene cluster | |
| 444 | Tailocin | MIC-14439 |
| | gene cluster | |
| 445 | Tailocin | MIC-14439 |
| | gene cluster | |
| 446 | Tailocin | MIC-14439 |
| | gene cluster | |
| 447 | Tailocin | MIC-14439 |
| | gene cluster | |
| 448 | Tailocin | MIC-14439 |
| | gene cluster | |
| 449 | Tailocin | MIC-14439 |
| | gene cluster | |
| 450 | Tailocin | MIC-14439 |
| | gene cluster | |
| 451 | Tailocin | MIC-14439 |
| | gene cluster | |
| 452 | Tailocin | MIC-14439 |
| | gene cluster | |
| 453 | Tailocin | MIC-87588, MIC-86605, MIC-54642 |
| | gene cluster | |
| 454 | Tailocin | MIC-87588, MIC-86605, MIC-54642 |
| | gene cluster | |
| 455 | Tailocin | MIC-87588, MIC-86605, MIC-54642 |
| | gene cluster | |
| 456 | Tailocin | MIC-87588, MIC-86605, MIC-54642, |
| | gene cluster | MIC-84492 |
| 457 | Tailocin | MIC-87588, MIC-86605, MIC-54642, |
| | gene cluster | MIC-84492 |
| 458 | Tailocin | MIC-87588, MIC-86605, MIC-54642, |
| | gene cluster | MIC-84492 |
| 459 | Tailocin | MIC-87588, MIC-54642 |
| | gene cluster | |
| 460 | Tailocin | MIC-79613, MIC-53518 |
| | gene cluster | |
| 461 | Tailocin | MIC-79613, MIC-53518 |
| | gene cluster | |
| 462 | Tailocin | MIC-79613, MIC-53518 |
| | gene cluster | |
| 463 | Tailocin | MIC-79613, MIC-53518 |
| | gene cluster | |
| 464 | Tailocin | MIC-79613, MIC-53518 |
| | gene cluster | |
| 465 | Tailocin | MIC-79613, MIC-53518 |
| | gene cluster | |
| 466 | Tailocin | MIC-79613, MIC-53518 |
| | gene cluster | |
| 467 | Tailocin | MIC-79613, MIC-53518 |
| | gene cluster | |
| 468 | Tailocin | MIC-79613, MIC-53518 |
| | gene cluster | |
| 469 | Tailocin | MIC-79613, MIC-53518 |
| | gene cluster | |
| 470 | Tailocin | MIC-79613, MIC-53518 |
| | gene cluster | |
| 471 | Tailocin | MIC-79613, MIC-53518 |
| | gene cluster | |
| 472 | Tailocin | MIC-79613, MIC-53518 |
| | gene cluster | |
| 473 | Tailocin | MIC-79613, MIC-53518 |
| | gene cluster | |
| 474 | Tailocin | MIC-79613, MIC-53518 |
| | gene cluster | |
| 475 | Tailocin | MIC-79613, MIC-53518 |
| | gene cluster | |
| 476 | Tailocin | MIC-79613, MIC-53518 |
| | gene cluster | |
| 477 | Tailocin | MIC-79613, MIC-53518 |
| | gene cluster | |
| 478 | Tailocin | MIC-79613, MIC-53518 |
| | gene cluster | |
| 479 | Tailocin | MIC-79613, MIC-53518 |
| | gene cluster | |
| 480 | Tailocin | MIC-79613, MIC-53518 |
| | gene cluster | |
| 481 | Tailocin | MIC-79613, MIC-53518 |
| | gene cluster | |
| 482 | Tailocin | MIC-79613, MIC-53518 |
| | gene cluster | |
| 483 | Tailocin | MIC-79613, MIC-53518 |
| | gene cluster | |
| 484 | Tailocin | MIC-79613, MIC-53518 |
| | gene cluster | |
| 485 | Tailocin | MIC-53518 |
| | gene cluster | |
| 486 | Tailocin | MIC-53518 |
| | gene cluster | |
| 487 | Tailocin | MIC-53518 |
| | gene cluster | |
| 488 | Tailocin | MIC-53518 |
| | gene cluster | |
| 489 | Tailocin | MIC-53518 |
| | gene cluster | |
| 490 | Tailocin | MIC-53518 |
| | gene cluster | |
| 491 | Tailocin | MIC-53518 |
| | gene cluster | |
| 492 | Tailocin | MIC-83010, MIC-94135, MIC-46385, |
| | gene cluster | MIC-62164, MIC-87084, MIC-36497, |
| | | MIC-82689 |
| 493 | Tailocin | MIC-83010, MIC-46385, MIC-62164 |
| | gene cluster | |
| 494 | Tailocin | MIC-83010, MIC-46385, MIC-62164, |
| | gene cluster | MIC-87084 |
| 495 | Tailocin | MIC-83010, MIC-46385, MIC-62164, |
| | gene cluster | MIC-87084 |
| 496 | Tailocin | MIC-83010, MIC-46385, MIC-62164, |
| | gene cluster | MIC-87084 |
| 497 | Tailocin | MIC-83740 |
| | gene cluster | |
| 498 | Tailocin | MIC-83740 |
| | gene cluster | |
| 499 | Tailocin | MIC-83740 |
| | gene cluster | |
| 500 | Tailocin | MIC-83740 |
| | gene cluster | |
| 501 | Tailocin | MIC-83740 |
| | gene cluster | |
| 502 | Tailocin | MIC-83740 |
| | gene cluster | |
| 503 | Tailocin | MIC-83740 |
| | gene cluster | |
| 504 | Tailocin | MIC-83740 |
| | gene cluster | |
| 505 | Tailocin | MIC-83740 |
| | gene cluster | |
| 506 | Tailocin | MIC-83740 |
| | gene cluster | |
| 507 | Tailocin | MIC-83740 |
| | gene cluster | |
| 508 | Tailocin | MIC-83740 |
| | gene cluster | |
| 509 | Tailocin | MIC-80455 |
| | gene cluster | |
| 510 | Tailocin | MIC-80455 |
| | gene cluster | |
| 511 | Tailocin | MIC-80455 |
| | gene cluster | |
| 512 | Tailocin | MIC-80455 |
| | gene cluster | |
| 513 | Tailocin | MIC-80455 |
| | gene cluster | |
| 514 | Tailocin | MIC-80455 |
| | gene cluster | |
| 515 | Tailocin | MIC-80455 |
| | gene cluster | |
| 516 | Tailocin | MIC-80455 |
| | gene cluster | |
| 517 | Tailocin | MIC-80455 |
| | gene cluster | |
| 518 | Tailocin | MIC-80455 |
| | gene cluster | |
| 519 | Tailocin | MIC-87588, MIC-86605, MIC-54642 |
| | gene cluster | |
| 520 | Tailocin | MIC-87588, MIC-86605, MIC-54642 |
| | gene cluster | |
| 521 | Tailocin | MIC-87588, MIC-86605, MIC-54642 |
| | gene cluster | |
| 522 | Tailocin | MIC-87588, MIC-86605, MIC-54642 |
| | gene cluster | |
| 523 | Tailocin | MIC-87588, MIC-86605, MIC-54642 |
| | gene cluster | |
| 524 | Tailocin | MIC-87588, MIC-86605, MIC-54642 |
| | gene cluster | |
| 525 | Tailocin | MIC-87588, MIC-86605, MIC-54642 |
| | gene cluster | |
| 526 | Tailocin | MIC-87588, MIC-86605, MIC-54642 |
| | gene cluster | |
| 527 | Tailocin | MIC-87588, MIC-86605, MIC-54642 |
| | gene cluster | |
| 528 | Tailocin | MIC-87588, MIC-86605, MIC-54642 |
| | gene cluster | |
| 529 | Tailocin | MIC-87588, MIC-86605, MIC-54642 |
| | gene cluster | |
| 530 | Tailocin | MIC-87588, MIC-86605, MIC-54642 |
| | gene cluster | |
| 531 | Tailocin | MIC-87588, MIC-86605, MIC-54642 |
| | gene cluster | |
| 532 | Tailocin | MIC-87588, MIC-86605, MIC-54642 |
| | gene cluster | |
| 533 | Tailocin | MIC-87588, MIC-86605, MIC-54642 |
| | gene cluster | |
| 534 | Tailocin | MIC-87588, MIC-86605, MIC-54642 |
| | gene cluster | |
| 535 | Tailocin | MIC-87588, MIC-86605, MIC-54642 |
| | gene cluster | |
| 536 | Tailocin | MIC-87588, MIC-86605, MIC-54642 |
| | gene cluster | |
| 537 | Tailocin | MIC-87588, MIC-86605, MIC-54642 |
| | gene cluster | |
| 538 | Tailocin | MIC-87588, MIC-86605, MIC-54642 |
| | gene cluster | |
| 539 | Tailocin | MIC-87588, MIC-86605, MIC-54642 |
| | gene cluster | |
| 540 | Tailocin | MIC-87588, MIC-86605, MIC-54642 |
| | gene cluster | |
| 541 | Tailocin | MIC-87588, MIC-86605, MIC-54642 |
| | gene cluster | |
| 542 | Tailocin | MIC-87588, MIC-86605, MIC-54642 |
| | gene cluster | |
| 543 | Tailocin | MIC-87588, MIC-86605, MIC-54642 |
| | gene cluster | |
| 544 | Tailocin | MIC-87588, MIC-86605, MIC-54642 |
| | gene cluster | |
| 545 | Tailocin | MIC-87588, MIC-86605, MIC-54642 |
| | gene cluster | |
| 546 | Tailocin | MIC-87588, MIC-86605, MIC-54642 |
| | gene cluster | |
| 547 | Tailocin | MIC-87588, MIC-86605, MIC-54642 |
| | gene cluster | |
| 548 | Tailocin | MIC-87588, MIC-86605, MIC-54642 |
| | gene cluster | |
| 549 | Tailocin | MIC-87588, MIC-86605, MIC-54642, |
| | gene cluster | MIC-84492 |
| 550 | Tailocin | MIC-87588, MIC-86605, MIC-54642, |
| | gene cluster | MIC-84492 |
| 551 | Tailocin | MIC-87588, MIC-86605, MIC-54642, |
| | gene cluster | MIC-84492 |
| 552 | Tailocin | MIC-87588, MIC-86605, MIC-54642, |
| | gene cluster | MIC-84492 |
| 553 | Tailocin | MIC-87588, MIC-86605, MIC-54642, |
| | gene cluster | MIC-84492 |
| 554 | Tailocin | MIC-87588, MIC-86605, MIC-54642, |
| | gene cluster | MIC-84492 |
| 555 | Tailocin | MIC-87588, MIC-54642 |
| | gene cluster | |
| 556 | Tailocin | MIC-86605 |
| | gene cluster | |
| 557 | Tailocin | MIC-86605 |
| | gene cluster | |
| 558 | Tailocin | MIC-86605, MIC-75437, MIC-14439, |
| | gene cluster | MIC-20446, MIC-94135, MIC-87084, |
| | | MIC-36497, MIC-82689 |
| 559 | Tailocin | MIC-86605, MIC-75437, MIC-20446, |
| | gene cluster | MIC-94135, MIC-36497, MIC-82689 |
| 560 | Tailocin | MIC-86605, MIC-94135, MIC-36497 |
| | gene cluster | |
| 561 | Tailocin | MIC-86605, MIC-94135, MIC-36497, |
| | gene cluster | MIC-82689 |
| 562 | Tailocin | MIC-86605, MIC-94135, MIC-36497, |
| | gene cluster | MIC-82689 |
| 563 | Tailocin | MIC-94504 |
| | gene cluster | |
| 564 | Tailocin | MIC-94504 |
| | gene cluster | |
| 565 | Tailocin | MIC-94504 |
| | gene cluster | |
| 566 | Tailocin | MIC-24837, MIC-55579 |
| | gene cluster | |
| 567 | Tailocin | MIC-24837, MIC-55579 |
| | gene cluster | |
| 568 | Tailocin | MIC-24837, MIC-55579 |
| | gene cluster | |
| 569 | Tailocin | MIC-24837, MIC-55579 |
| | gene cluster | |
| 570 | Tailocin | MIC-24837, MIC-55579 |
| | gene cluster | |
| 571 | Tailocin | MIC-24837, MIC-55579 |
| | gene cluster | |
| 572 | Tailocin | MIC-24837, MIC-55579 |
| | gene cluster | |
| 573 | Tailocin | MIC-24837, MIC-55579 |
| | gene cluster | |
| 574 | Tailocin | MIC-24837, MIC-55579 |
| | gene cluster | |
| 575 | Tailocin | MIC-24837, MIC-55579 |
| | gene cluster | |
| 576 | Tailocin | MIC-24837, MIC-55579 |
| | gene cluster | |
| 577 | Tailocin | MIC-24837, MIC-55579 |
| | gene cluster | |
| 578 | Tailocin | MIC-24837, MIC-55579 |
| | gene cluster | |
| 579 | Tailocin | MIC-24837, MIC-55579 |
| | gene cluster | |
| 580 | Tailocin | MIC-24837, MIC-55579 |
| | gene cluster | |
| 581 | Tailocin | MIC-24837, MIC-55579 |
| | gene cluster | |
| 582 | Tailocin | MIC-24837, MIC-55579 |
| | gene cluster | |
| 583 | Tailocin | MIC-24837, MIC-55579 |
| | gene cluster | |
| 584 | Tailocin | MIC-24837, MIC-55579 |
| | gene cluster | |
| 585 | Tailocin | MIC-24837, MIC-55579 |
| | gene cluster | |
| 586 | Tailocin | MIC-24837, MIC-55579 |
| | gene cluster | |
| 587 | Tailocin | MIC-24837, MIC-55579 |
| | gene cluster | |
| 588 | Tailocin | MIC-24837, MIC-55579 |
| | gene cluster | |
| 589 | Tailocin | MIC-24837, MIC-55579 |
| | gene cluster | |
| 590 | Tailocin | MIC-24837, MIC-55579 |
| | gene cluster | |
| 591 | Tailocin | MIC-24837, MIC-55579 |
| | gene cluster | |
| 592 | Tailocin | MIC-24837, MIC-55579 |
| | gene cluster | |
| 593 | Tailocin | MIC-24837, MIC-55579 |
| | gene cluster | |
| 594 | Tailocin | MIC-24837, MIC-55579 |
| | gene cluster | |
| 595 | Tailocin | MIC-24837, MIC-55579 |
| | gene cluster | |
| 596 | Tailocin | MIC-24837, MIC-55579 |
| | gene cluster | |
| 597 | Tailocin | MIC-24837, MIC-55579 |
| | gene cluster | |
| 598 | Tailocin | MIC-24837, MIC-55579 |
| | gene cluster | |
| 599 | Tailocin | MIC-24837, MIC-55579 |
| | gene cluster | |
| 600 | Tailocin | MIC-24837, MIC-55579 |
| | gene cluster | |
| 601 | Tailocin | MIC-24837, MIC-55579 |
| | gene cluster | |
| 602 | Tailocin | MIC-24837, MIC-55579 |
| | gene cluster | |
| 603 | Tailocin | MIC-24837, MIC-55579, MIC-85267 |
| | gene cluster | |
| 604 | Tailocin | MIC-24837, MIC-55579, MIC-85267 |
| | gene cluster | |
| 605 | Tailocin | MIC-24837, MIC-55579, MIC-29285 |
| | gene cluster | |
| 606 | Tailocin | MIC-55579 |
| | gene cluster | |
| 607 | Tailocin | MIC-87198, MIC-94458, MIC-30352, |
| | gene cluster | MIC-82867 |
| 608 | Tailocin | MIC-19845 |
| | gene cluster | |
| 609 | Tailocin | MIC-19845 |
| | gene cluster | |
| 610 | Tailocin | MIC-19845 |
| | gene cluster | |
| 611 | Tailocin | MIC-19845 |
| | gene cluster | |
| 612 | Tailocin | MIC-19845 |
| | gene cluster | |
| 613 | Tailocin | MIC-19845 |
| | gene cluster | |
| 614 | Tailocin | MIC-19845 |
| | gene cluster | |
| 615 | Tailocin | MIC-19845 |
| | gene cluster | |
| 616 | Tailocin | MIC-19845 |
| | gene cluster | |
| 617 | Tailocin | MIC-19845 |
| | gene cluster | |
| 618 | Tailocin | MIC-19845 |
| | gene cluster | |
| 619 | Tailocin | MIC-84492 |
| | gene cluster | |
| 620 | Tailocin | MIC-84492 |
| | gene cluster | |
| 621 | Tailocin | MIC-84492 |
| | gene cluster | |
| 622 | Tailocin | MIC-84492 |
| | gene cluster | |
| 623 | Tailocin | MIC-84492 |
| | gene cluster | |
| 624 | Tailocin | MIC-84492 |
| | gene cluster | |
| 625 | Tailocin | MIC-84492 |
| | gene cluster | |
| 626 | Tailocin | MIC-84492 |
| | gene cluster | |
| 627 | Tailocin | MIC-84492 |
| | gene cluster | |
| 628 | Tailocin | MIC-84492 |
| | gene cluster | |
| 629 | Tailocin | MIC-84492 |
| | gene cluster | |
| 630 | Tailocin | MIC-84492 |
| | gene cluster | |
| 631 | Tailocin | MIC-84492 |
| | gene cluster | |
| 632 | Tailocin | MIC-84492 |
| | gene cluster | |
| 633 | Tailocin | MIC-84492 |
| | gene cluster | |
| 634 | Tailocin | MIC-84492 |
| | gene cluster | |
| 635 | Tailocin | MIC-84492 |
| | gene cluster | |
| 636 | Tailocin | MIC-84492 |
| | gene cluster | |
| 637 | Tailocin | MIC-84492 |
| | gene cluster | |
| 638 | Tailocin | MIC-84492 |
| | gene cluster | |
| 639 | Tailocin | MIC-84492 |
| | gene cluster | |
| 640 | Tailocin | MIC-84492 |
| | gene cluster | |
| 641 | Tailocin | MIC-84492 |
| | gene cluster | |
| 642 | Tailocin | MIC-84492, MIC-85267 |
| | gene cluster | |
| 643 | Tailocin | MIC-50391, MIC-69701 |
| | gene cluster | |
| 644 | Tailocin | MIC-50391, MIC-69701 |
| | gene cluster | |
| 645 | Tailocin | MIC-50391, MIC-69701 |
| | gene cluster | |
| 646 | Tailocin | MIC-50391, MIC-69701 |
| | gene cluster | |
| 647 | Tailocin | MIC-50391, MIC-69701 |
| | gene cluster | |
| 648 | Tailocin | MIC-50391, MIC-69701 |
| | gene cluster | |
| 649 | Tailocin | MIC-50391, MIC-69701 |
| | gene cluster | |
| 650 | Tailocin | MIC-50391, MIC-69701 |
| | gene cluster | |
| 651 | Tailocin | MIC-50391, MIC-69701 |
| | gene cluster | |
| 652 | Tailocin | MIC-50391, MIC-69701 |
| | gene cluster | |
| 653 | Tailocin | MIC-50391, MIC-69701 |
| | gene cluster | |
| 654 | Tailocin | MIC-50391, MIC-69701 |
| | gene cluster | |
| 655 | Tailocin | MIC-85267 |
| | gene cluster | |
| 656 | Tailocin | MIC-85267 |
| | gene cluster | |
| 657 | Tailocin | MIC-85267 |
| | gene cluster | |
| 658 | Tailocin | MIC-85267 |
| | gene cluster | |
| 659 | Tailocin | MIC-85267 |
| | gene cluster | |
| 660 | Tailocin | MIC-85267 |
| | gene cluster | |
| 661 | Tailocin | MIC-85267 |
| | gene cluster | |
| 662 | Tailocin | MIC-85267 |
| | gene cluster | |
| 663 | Tailocin | MIC-85267 |
| | gene cluster | |
| 664 | Tailocin | MIC-85267 |
| | gene cluster | |
| 665 | Tailocin | MIC-85267 |
| | gene cluster | |
| 666 | Tailocin | MIC-85267 |
| | gene cluster | |
| 667 | Tailocin | MIC-85267 |
| | gene cluster | |
| 668 | Tailocin | MIC-85267 |
| | gene cluster | |
| 669 | Tailocin | MIC-85267 |
| | gene cluster | |
| 670 | Tailocin | MIC-85267 |
| | gene cluster | |
| 671 | Tailocin | MIC-85267 |
| | gene cluster | |
| 672 | Tailocin | MIC-85267 |
| | gene cluster | |
| 673 | Tailocin | MIC-85267 |
| | gene cluster | |
| 674 | Tailocin | MIC-85267 |
| | gene cluster | |
| 675 | Tailocin | MIC-85267 |
| | gene cluster | |
| 676 | Tailocin | MIC-85267 |
| | gene cluster | |
| 677 | Tailocin | MIC-85267 |
| | gene cluster | |
| 678 | Tailocin | MIC-85267 |
| | gene cluster | |
| 679 | Tailocin | MIC-85267 |
| | gene cluster | |
| 680 | Tailocin | MIC-85267 |
| | gene cluster | |
| 681 | Tailocin | MIC-85267 |
| | gene cluster | |
| 682 | Tailocin | MIC-85267 |
| | gene cluster | |
| 683 | Tailocin | MIC-85267 |
| | gene cluster | |
| 684 | Tailocin | MIC-85267 |
| | gene cluster | |
| 685 | Tailocin | MIC-85267 |
| | gene cluster | |
| 686 | Tailocin | MIC-85267 |
| | gene cluster | |
| 687 | Tailocin | MIC-85267 |
| | gene cluster | |
| 688 | Tailocin | MIC-85267 |
| | gene cluster | |
| 689 | Tailocin | MIC-85267 |
| | gene cluster | |
| 690 | Tailocin | MIC-85267 |
| | gene cluster | |
| 691 | Tailocin | MIC-85267 |
| | gene cluster | |
| 692 | Tailocin | MIC-85267 |
| | gene cluster | |
| 693 | Tailocin | MIC-85267 |
| | gene cluster | |
| 694 | Tailocin | MIC-85267 |
| | gene cluster | |
| 695 | Tailocin | MIC-85267 |
| | gene cluster | |
| 696 | Tailocin | MIC-85267 |
| | gene cluster | |
| 697 | Tailocin | MIC-75437, MIC-20446 |
| | gene cluster | |
| 698 | Tailocin | MIC-75437, MIC-20446 |
| | gene cluster | |
| 699 | Tailocin | MIC-75437, MIC-20446 |
| | gene cluster | |
| 700 | Tailocin | MIC-75437, MIC-20446 |
| | gene cluster | |
| 701 | Tailocin | MIC-75437, MIC-20446 |
| | gene cluster | |
| 702 | Tailocin | MIC-75437, MIC-20446 |
| | gene cluster | |
| 703 | Tailocin | MIC-75437, MIC-20446 |
| | gene cluster | |
| 704 | Tailocin | MIC-75437, MIC-20446 |
| | gene cluster | |
| 705 | Tailocin | MIC-75437, MIC-20446 |
| | gene cluster | |
| 706 | Tailocin | MIC-75437, MIC-20446 |
| | gene cluster | |
| 707 | Tailocin | MIC-75437, MIC-20446 |
| | gene cluster | |
| 708 | Tailocin | MIC-75437, MIC-20446 |
| | gene cluster | |
| 709 | Tailocin | MIC-75437, MIC-20446 |
| | gene cluster | |
| 710 | Tailocin | MIC-75437, MIC-20446 |
| | gene cluster | |
| 711 | Tailocin | MIC-38993 |
| | gene cluster | |
| 712 | Tailocin | MIC-38993 |
| | gene cluster | |
| 713 | Tailocin | MIC-38993 |
| | gene cluster | |
| 714 | Tailocin | MIC-38993 |
| | gene cluster | |
| 715 | Tailocin | MIC-38993 |
| | gene cluster | |
| 716 | Tailocin | MIC-38993 |
| | gene cluster | |
| 717 | Tailocin | MIC-38993 |
| | gene cluster | |
| 718 | Tailocin | MIC-38993 |
| | gene cluster | |
| 719 | Tailocin | MIC-38993 |
| | gene cluster | |
| 720 | Tailocin | MIC-38993 |
| | gene cluster | |
| 721 | Tailocin | MIC-38993 |
| | gene cluster | |
| 722 | Tailocin | MIC-38993 |
| | gene cluster | |
| 723 | Tailocin | MIC-38993 |
| | gene cluster | |
| 724 | Tailocin | MIC-38993 |
| | gene cluster | |
| 725 | Tailocin | MIC-38993 |
| | gene cluster | |
| 726 | Tailocin | MIC-38993 |
| | gene cluster | |
| 727 | Tailocin | MIC-38993 |
| | gene cluster | |
| 728 | Tailocin | MIC-38993, MIC-94458, MIC-30352, |
| | gene cluster | MIC-82867 |
| 729 | Tailocin | MIC-94135 |
| | gene cluster | |
| 730 | Tailocin | MIC-94135 |
| | gene cluster | |
| 731 | Tailocin | MIC-94135 |
| | gene cluster | |
| 732 | Tailocin | MIC-94135 |
| | gene cluster | |
| 733 | Tailocin | MIC-94135 |
| | gene cluster | |
| 734 | Tailocin | MIC-94135 |
| | gene cluster | |
| 735 | Tailocin | MIC-94135 |
| | gene cluster | |
| 736 | Tailocin | MIC-94135 |
| | gene cluster | |
| 737 | Tailocin | MIC-94135 |
| | gene cluster | |
| 738 | Tailocin | MIC-94135 |
| | gene cluster | |
| 739 | Tailocin | MIC-94135 |
| | gene cluster | |
| 740 | Tailocin | MIC-94458, MIC-30352, MIC-82867 |
| | gene cluster | |
| 741 | Tailocin | MIC-94458, MIC-30352, MIC-82867 |
| | gene cluster | |
| 742 | Tailocin | MIC-94458, MIC-30352, MIC-82867 |
| | gene cluster | |
| 743 | Tailocin | MIC-94458, MIC-30352, MIC-82867 |
| | gene cluster | |
| 744 | Tailocin | MIC-94458, MIC-30352, MIC-82867 |
| | gene cluster | |
| 745 | Tailocin | MIC-94458, MIC-30352, MIC-82867 |
| | gene cluster | |
| 746 | Tailocin | MIC-94458, MIC-30352, MIC-82867 |
| | gene cluster | |
| 747 | Tailocin | MIC-94458, MIC-30352, MIC-82867 |
| | gene cluster | |
| 748 | Tailocin | MIC-94458, MIC-30352, MIC-82867 |
| | gene cluster | |
| 749 | Tailocin | MIC-94458, MIC-30352, MIC-82867 |
| | gene cluster | |
| 750 | Tailocin | MIC-94458, MIC-30352, MIC-82867 |
| | gene cluster | |
| 751 | Tailocin | MIC-94458, MIC-30352, MIC-82867 |
| | gene cluster | |
| 752 | Tailocin | MIC-94458, MIC-30352, MIC-82867 |
| | gene cluster | |
| 753 | Tailocin | MIC-94458, MIC-30352, MIC-82867 |
| | gene cluster | |
| 754 | Tailocin | MIC-94458, MIC-30352, MIC-82867 |
| | gene cluster | |
| 755 | Tailocin | MIC-94458, MIC-30352, MIC-82867 |
| | gene cluster | |
| 756 | Tailocin | MIC-94458, MIC-30352, MIC-82867 |
| | gene cluster | |
| 757 | Tailocin | MIC-94458, MIC-30352, MIC-82867 |
| | gene cluster | |
| 758 | Tailocin | MIC-94458, MIC-30352, MIC-82867 |
| | gene cluster | |
| 759 | Tailocin | MIC-94458, MIC-30352, MIC-82867 |
| | gene cluster | |
| 760 | Tailocin | MIC-94458, MIC-30352, MIC-82867 |
| | gene cluster | |
| 761 | Tailocin | MIC-94458, MIC-30352, MIC-82867 |
| | gene cluster | |
| 762 | Tailocin | MIC-94458, MIC-30352, MIC-82867 |
| | gene cluster | |
| 763 | Tailocin | MIC-94458, MIC-30352, MIC-82867 |
| | gene cluster | |
| 764 | Tailocin | MIC-94458, MIC-30352, MIC-82867 |
| | gene cluster | |
| 765 | Tailocin | MIC-94458, MIC-30352, MIC-82867 |
| | gene cluster | |
| 766 | Tailocin | MIC-94458, MIC-30352, MIC-82867 |
| | gene cluster | |
| 767 | Tailocin | MIC-94458, MIC-30352, MIC-82867 |
| | gene cluster | |
| 768 | Tailocin | MIC-94458, MIC-30352, MIC-82867 |
| | gene cluster | |
| 769 | Tailocin | MIC-94458, MIC-30352, MIC-82867 |
| | gene cluster | |
| 770 | Tailocin | MIC-94458, MIC-30352, MIC-82867 |
| | gene cluster | |
| 771 | Tailocin | MIC-94458, MIC-30352, MIC-82867 |
| | gene cluster | |
| 772 | Tailocin | MIC-94458, MIC-30352, MIC-82867 |
| | gene cluster | |
| 773 | Tailocin | MIC-94458, MIC-30352, MIC-82867 |
| | gene cluster | |
| 774 | Tailocin | MIC-94458, MIC-30352, MIC-82867 |
| | gene cluster | |
| 775 | Tailocin | MIC-94458, MIC-30352, MIC-82867 |
| | gene cluster | |
| 776 | Tailocin | MIC-94458, MIC-30352, MIC-82867 |
| | gene cluster | |
| 777 | Tailocin | MIC-94458, MIC-30352, MIC-82867 |
| | gene cluster | |
| 778 | Tailocin | MIC-94458, MIC-30352, MIC-82867 |
| | gene cluster | |
| 779 | Tailocin | MIC-94458, MIC-30352, MIC-82867 |
| | gene cluster | |
| 780 | Tailocin | MIC-94458, MIC-30352, MIC-82867 |
| | gene cluster | |
| 781 | Tailocin | MIC-94458, MIC-30352, MIC-82867 |
| | gene cluster | |
| 782 | Tailocin | MIC-46385, MIC-62164 |
| | gene cluster | |
| 783 | Tailocin | MIC-46385, MIC-62164 |
| | gene cluster | |
| 784 | Tailocin | MIC-46385, MIC-62164 |
| | gene cluster | |
| 785 | Tailocin | MIC-46385, MIC-62164 |
| | gene cluster | |
| 786 | Tailocin | MIC-46385, MIC-62164 |
| | gene cluster | |
| 787 | Tailocin | MIC-46385, MIC-62164 |
| | gene cluster | |
| 788 | Tailocin | MIC-46385, MIC-62164 |
| | gene cluster | |
| 789 | Tailocin | MIC-46385, MIC-62164 |
| | gene cluster | |
| 790 | Tailocin | MIC-46385, MIC-62164 |
| | gene cluster | |
| 791 | Tailocin | MIC-36254 |
| | gene cluster | |
| 792 | Tailocin | MIC-36254 |
| | gene cluster | |
| 793 | Tailocin | MIC-36254 |
| | gene cluster | |
| 794 | Tailocin | MIC-36254 |
| | gene cluster | |
| 795 | Tailocin | MIC-36254 |
| | gene cluster | |
| 796 | Tailocin | MIC-14970, MIC-11290 |
| | gene cluster | |
| 797 | Tailocin | MIC-14970, MIC-11290 |
| | gene cluster | |
| 798 | Tailocin | MIC-14970, MIC-11290 |
| | gene cluster | |
| 799 | Tailocin | MIC-14970, MIC-11290 |
| | gene cluster | |
| 800 | Tailocin | MIC-14970, MIC-11290 |
| | gene cluster | |
| 801 | Tailocin | MIC-14970, MIC-11290 |
| | gene cluster | |
| 802 | Tailocin | MIC-14970, MIC-11290 |
| | gene cluster | |
| 803 | Tailocin | MIC-14970, MIC-11290 |
| | gene cluster | |
| 804 | Tailocin | MIC-14970, MIC-11290 |
| | gene cluster | |
| 805 | Tailocin | MIC-14970, MIC-11290 |
| | gene cluster | |
| 806 | Tailocin | MIC-14970, MIC-11290 |
| | gene cluster | |
| 807 | Tailocin | MIC-14970, MIC-11290 |
| | gene cluster | |
| 808 | Tailocin | MIC-14970, MIC-11290 |
| | gene cluster | |
| 809 | Tailocin | MIC-14970, MIC-11290 |
| | gene cluster | |
| 810 | Tailocin | MIC-14970, MIC-11290 |
| | gene cluster | |
| 811 | Tailocin | MIC-14970, MIC-11290 |
| | gene cluster | |
| 812 | Tailocin | MIC-14970, MIC-11290 |
| | gene cluster | |
| 813 | Tailocin | MIC-14970, MIC-11290 |
| | gene cluster | |
| 814 | Tailocin | MIC-14970, MIC-11290 |
| | gene cluster | |
| 815 | Tailocin | MIC-14970, MIC-11290 |
| | gene cluster | |
| 816 | Tailocin | MIC-14970, MIC-11290 |
| | gene cluster | |
| 817 | Tailocin | MIC-14970, MIC-11290 |
| | gene cluster | |
| 818 | Tailocin | MIC-87084 |
| | gene cluster | |
| 819 | Tailocin | MIC-87084 |
| | gene cluster | |
| 820 | Tailocin | MIC-87084 |
| | gene cluster | |
| 821 | Tailocin | MIC-87084 |
| | gene cluster | |
| 822 | Tailocin | MIC-87084 |
| | gene cluster | |
| 823 | Tailocin | MIC-87084 |
| | gene cluster | |
| 824 | Tailocin | MIC-87084 |
| | gene cluster | |
| 825 | Tailocin | MIC-36497 |
| | gene cluster | |
| 826 | Tailocin | MIC-36497 |
| | gene cluster | |
| 827 | Tailocin | MIC-36497 |
| | gene cluster | |
| 828 | Tailocin | MIC-36497 |
| | gene cluster | |
| 829 | Tailocin | MIC-36497 |
| | gene cluster | |
| 830 | Tailocin | MIC-36497 |
| | gene cluster | |
| 831 | Tailocin | MIC-36497 |
| | gene cluster | |
| 832 | Tailocin | MIC-36497 |
| | gene cluster | |
| 833 | Tailocin | MIC-36497 |
| | gene cluster | |
| 834 | Tailocin | MIC-36497 |
| | gene cluster | |
| 835 | Tailocin | MIC-36497 |
| | gene cluster | |
| 836 | Tailocin | MIC-36497 |
| | gene cluster | |
| 837 | Tailocin | MIC-36497 |
| | gene cluster | |
| 838 | Tailocin | MIC-36497 |
| | gene cluster | |
| 839 | Tailocin | MIC-36497 |
| | gene cluster | |
| 840 | Tailocin | MIC-36497 |
| | gene cluster | |
| 841 | Tailocin | MIC-36497 |
| | gene cluster | |
| 842 | Tailocin | MIC-36497 |
| | gene cluster | |
| 843 | Tailocin | MIC-36497 |
| | gene cluster | |
| 844 | Tailocin | MIC-29285 |
| | gene cluster | |
| 845 | Tailocin | MIC-29285 |
| | gene cluster | |
| 846 | Tailocin | MIC-29285 |
| | gene cluster | |
| 847 | Tailocin | MIC-29285 |
| | gene cluster | |
| 848 | Tailocin | MIC-29285 |
| | gene cluster | |
| 849 | Tailocin | MIC-29285 |
| | gene cluster | |
| 850 | Tailocin | MIC-29285 |
| | gene cluster | |
| 851 | Tailocin | MIC-29285 |
| | gene cluster | |
| 852 | Tailocin | MIC-29285 |
| | gene cluster | |
| 853 | Tailocin | MIC-29285 |
| | gene cluster | |
| 854 | Tailocin | MIC-29285 |
| | gene cluster | |
| 855 | Tailocin | MIC-29285 |
| | gene cluster | |
| 856 | Tailocin | MIC-29285 |
| | gene cluster | |
| 857 | Tailocin | MIC-29285 |
| | gene cluster | |
| 858 | Tailocin | MIC-29285 |
| | gene cluster | |
| 859 | Tailocin | MIC-29285 |
| | gene cluster | |
| 860 | Tailocin | MIC-29285 |
| | gene cluster | |
| 861 | Tailocin | MIC-29285 |
| | gene cluster | |
| 862 | Tailocin | MIC-29285 |
| | gene cluster | |
| 863 | Tailocin | MIC-29285 |
| | gene cluster | |
| 864 | Tailocin | MIC-29285 |
| | gene cluster | |
| 865 | Tailocin | MIC-29285 |
| | gene cluster | |
| 866 | Tailocin | MIC-29285 |
| | gene cluster | |
| 867 | Tailocin | MIC-29285 |
| | gene cluster | |
| 868 | Tailocin | MIC-29285 |
| | gene cluster | |
| 869 | Tailocin | MIC-29285 |
| | gene cluster | |
| 870 | Tailocin | MIC-29285 |
| | gene cluster | |
| 871 | Tailocin | MIC-29285 |
| | gene cluster | |
| 872 | Tailocin | MIC-29285 |
| | gene cluster | |
| 873 | Tailocin | MIC-29285 |
| | gene cluster | |
| 874 | Tailocin | MIC-29285 |
| | gene cluster | |
| 875 | Tailocin | MIC-29285 |
| | gene cluster | |
| 876 | Tailocin | MIC-29285 |
| | gene cluster | |
| 877 | Tailocin | MIC-29285 |
| | gene cluster | |
| 878 | Tailocin | MIC-29285 |
| | gene cluster | |
| 879 | Tailocin | MIC-29285 |
| | gene cluster | |
| 880 | Tailocin | MIC-29285 |
| | gene cluster | |
| 881 | Tailocin | MIC-29285 |
| | gene cluster | |
| 882 | Tailocin | MIC-29285 |
| | gene cluster | |
| 883 | Tailocin | MIC-29285 |
| | gene cluster | |
| 884 | Tailocin | MIC-29285 |
| | gene cluster | |
| 885 | Tailocin | MIC-29285 |
| | gene cluster | |
| 886 | Tailocin | MIC-29285 |
| | gene cluster | |
| 887 | Tailocin | MIC-29285 |
| | gene cluster | |
| 888 | Tailocin | MIC-29285 |
| | gene cluster | |
| 889 | Tailocin | MIC-29285 |
| | gene cluster | |
| 890 | Tailocin | MIC-29285 |
| | gene cluster | |
| 891 | Tailocin | MIC-29285 |
| | gene cluster | |
| 892 | Tailocin | MIC-29285 |
| | gene cluster | |
| 893 | Tailocin | MIC-29285 |
| | gene cluster | |
| 894 | Tailocin | MIC-29285 |
| | gene cluster | |
| 895 | Tailocin | MIC-29285 |
| | gene cluster | |
| 896 | Tailocin | MIC-29285 |
| | gene cluster | |
| 897 | Tailocin | MIC-29285 |
| | gene cluster | |
| 898 | Tailocin | MIC-29285 |
| | gene cluster | |
| 899 | Tailocin | MIC-29285 |
| | gene cluster | |
| 900 | Tailocin | MIC-29285 |
| | gene cluster | |
| 901 | Tailocin | MIC-29285 |
| | gene cluster | |
| 902 | Tailocin | MIC-29285 |
| | gene cluster | |
| 903 | Tailocin | MIC-29285 |
| | gene cluster | |
| 904 | Tailocin | MIC-29285 |
| | gene cluster | |
| 905 | Tailocin | MIC-29285 |
| | gene cluster | |
| 906 | Tailocin | MIC-29285 |
| | gene cluster | |
| 907 | Tailocin | MIC-29285 |
| | gene cluster | |
| 908 | Tailocin | MIC-82689 |
| | gene cluster | |
| 909 | Tailocin | MIC-82689 |
| | gene cluster | |
| 910 | Tailocin | MIC-82689 |
| | gene cluster | |
| 911 | Tailocin | MIC-82689 |
| | gene cluster | |
| 912 | Tailocin | MIC-82689 |
| | gene cluster | |
| 913 | Tailocin | MIC-82689 |
| | gene cluster | |
| 914 | Tailocin | MIC-82689 |
| | gene cluster | |
| 915 | Tailocin | MIC-82689 |
| | gene cluster | |
| 916 | Tailocin | MIC-82689 |
| | gene cluster | |
| 917 | Tailocin | MIC-82689 |
| | gene cluster | |
| 918 | Tailocin | MIC-82689 |
| | gene cluster | |
| 919 | Tailocin | MIC-82689 |
| | gene cluster | |
| 920 | Tailocin | MIC-82689 |
| | gene cluster | |
| 921 | Tailocin | MIC-79613 |
| | gene cluster | |
| 922 | Tailocin | MIC-79613 |
| | gene cluster | |
| 923 | Tailocin | MIC-79613 |
| | gene cluster | |
| 924 | Tailocin | MIC-79613 |
| | gene cluster | |
| 925 | Tailocin | MIC-79613 |
| | gene cluster | |
| 926 | Tailocin | MIC-79613 |
| | gene cluster | |
| 927 | Tailocin | MIC-79613, MIC-53518 |
| | gene cluster | |
| 928 | Tailocin | MIC-79613, MIC-53518 |
| | gene cluster | |
| 929 | Tailocin | MIC-79613, MIC-53518 |
| | gene cluster | |
| 930 | Tailocin | MIC-79613, MIC-53518 |
| | gene cluster | |
| 931 | Tailocin | MIC-79613, MIC-53518 |
| | gene cluster | |
| 932 | Tailocin | MIC-79613, MIC-53518 |
| | gene cluster | |
| 933 | Tailocin | MIC-79613, MIC-53518 |
| | gene cluster | |
| 934 | Type VI | MIC-87588, MIC-86605, MIC-54642, |
| | secretion | MIC-61954, MIC-29662 |
| | system | |
| 935 | Type VI | MIC-87588, MIC-86605, MIC-54642, |
| | secretion | MIC-61954, MIC-29662 |
| | system | |
| 936 | Type VI | MIC-87588, MIC-86605, MIC-54642, |
| | secretion | MIC-61954, MIC-29662 |
| | system | |
| 937 | Type VI | MIC-87588, MIC-86605, MIC-54642, |
| | secretion | MIC-61954, MIC-29662 |
| | system | |
| 938 | Type VI | MIC-70076, MIC-73019, MIC-73547 |
| | secretion | |
| | system | |
| 939 | Type VI | MIC-70076, MIC-73019, MIC-73547 |
| | secretion | |
| | system | |
| 940 | Type VI | MIC-70076, MIC-73019, MIC-73547 |
| | secretion | |
| | system | |
| 941 | Type VI | MIC-70076, MIC-73019, MIC-73547 |
| | secretion | |
| | system | |
| 942 | Type VI | MIC-68901, MIC-19814 |
| | secretion | |
| | system | |
| 943 | Type VI | MIC-68773, MIC-14970, MIC-11290 |
| | secretion | |
| | system | |
| 944 | Type VI | MIC-81265, MIC-88834 |
| | secretion | |
| | system | |
| 945 | Type VI | MIC-68773 |
| | secretion | |
| | system | |
| 946 | Type VI | MIC-68773 |
| | secretion | |
| | system | |
| 947 | Type VI | MIC-68773 |
| | secretion | |
| | system | |
| 948 | Type VI | MIC-54778 |
| | secretion | |
| | system | |
| 949 | Type VI | MIC-54778 |
| | secretion | |
| | system | |
| 950 | Type VI | MIC-54778 |
| | secretion | |
| | system | |
| 951 | Type VI | MIC-54778 |
| | secretion | |
| | system | |
| 952 | Type VI | MIC-87198 |
| | secretion | |
| | system | |
| 953 | Type VI | MIC-87198 |
| | secretion | |
| | system | |
| 954 | Type VI | MIC-87198 |
| | secretion | |
| | system | |
| 955 | Type VI | MIC-87198 |
| | secretion | |
| | system | |
| 956 | Type VI | MIC-90405 |
| | secretion | |
| | system | |
| 957 | Type VI | MIC-90405 |
| | secretion | |
| | system | |
| 958 | Type VI | MIC-90405 |
| | secretion | |
| | system | |
| 959 | Type VI | MIC-14439 |
| | secretion | |
| | system | |
| 960 | Type VI | MIC-14439 |
| | secretion | |
| | system | |
| 961 | Type VI | MIC-14439 |
| | secretion | |
| | system | |
| 962 | Type VI | MIC-14439 |
| | secretion | |
| | system | |
| 963 | Type VI | MIC-79613, MIC-53518 |
| | secretion | |
| | system | |
| 964 | Type VI | MIC-83010 |
| | secretion | |
| | system | |
| 965 | Type VI | MIC-83740 |
| | secretion | |
| | system | |
| 966 | Type VI | MIC-80455 |
| | secretion | |
| | system | |
| 967 | Type VI | MIC-80455 |
| | secretion | |
| | system | |
| 968 | Type VI | MIC-80455 |
| | secretion | |
| | system | |
| 969 | Type VI | MIC-80455 |
| | secretion | |
| | system | |
| 970 | Type VI | MIC-94504 |
| | secretion | |
| | system | |
| 971 | Type VI | MIC-94504 |
| | secretion | |
| | system | |
| 972 | Type VI | MIC-94504 |
| | secretion | |
| | system | |
| 973 | Type VI | MIC-94504 |
| | secretion | |
| | system | |
| 974 | Type VI | MIC-24837, MIC-55579 |
| | secretion | |
| | system | |
| 975 | Type VI | MIC-24837, MIC-55579 |
| | secretion | |
| | system | |
| 976 | Type VI | MIC-24837, MIC-55579 |
| | secretion | |
| | system | |
| 977 | Type VI | MIC-24837, MIC-55579 |
| | secretion | |
| | system | |
| 978 | Type VI | MIC-19845 |
| | secretion | |
| | system | |
| 979 | Type VI | MIC-84492 |
| | secretion | |
| | system | |
| 980 | Type VI | MIC-84492 |
| | secretion | |
| | system | |
| 981 | Type VI | MIC-84492 |
| | secretion | |
| | system | |
| 982 | Type VI | MIC-50391, MIC-69701, MIC-52924 |
| | secretion | |
| | system | |
| 983 | Type VI | MIC-50391, MIC-69701, MIC-52924 |
| | secretion | |
| | system | |
| 984 | Type VI | MIC-50391, MIC-69701, MIC-52924 |
| | secretion | |
| | system | |
| 985 | Type VI | MIC-50391, MIC-69701, MIC-52924 |
| | secretion | |
| | system | |
| 986 | Type VI | MIC-85267 |
| | secretion | |
| | system | |
| 987 | Type VI | MIC-90405, MIC-82689 |
| | secretion | |
| | system | |
| 988 | Type VI | MIC-75437, MIC-20446 |
| | secretion | |
| | system | |
| 989 | Type VI | MIC-38993 |
| | secretion | |
| | system | |
| 990 | Type VI | MIC-38993 |
| | secretion | |
| | system | |
| 991 | Type VI | MIC-38993 |
| | secretion | |
| | system | |
| 992 | Type VI | MIC-38993 |
| | secretion | |
| | system | |
| 993 | Type VI | MIC-94135 |
| | secretion | |
| | system | |
| 994 | Type VI | MIC-94458, MIC-30352, MIC-82867 |
| | secretion | |
| | system | |
| 995 | Type VI | MIC-94458, MIC-30352, MIC-82867 |
| | secretion | |
| | system | |
| 996 | Type VI | MIC-94458, MIC-30352, MIC-82867 |
| | secretion | |
| | system | |
| 997 | Type VI | MIC-94458, MIC-30352, MIC-82867 |
| | secretion | |
| | system | |
| 998 | Type VI | MIC-46385, MIC-62164, MIC-87894 |
| | secretion | |
| | system | |
| 999 | Type VI | MIC-46385, MIC-62164, MIC-87894 |
| | secretion | |
| | system | |
| 1000 | Type VI | MIC-46385, MIC-62164, MIC-87894 |
| | secretion | |
| | system | |
| 1001 | Type VI | MIC-46385, MIC-62164, MIC-87894 |
| | secretion | |
| | system | |
| 1002 | Type VI | MIC-36254 |
| | secretion | |
| | system | |
| 1003 | Type VI | MIC-14970, MIC-11290 |
| | secretion | |
| | system | |
| 1004 | Type VI | MIC-14970, MIC-11290 |
| | secretion | |
| | system | |
| 1005 | Type VI | MIC-14970, MIC-11290 |
| | secretion | |
| | system | |
| 1006 | Type VI | MIC-87084 |
| | secretion | |
| | system | |
| 1007 | Type VI | MIC-36497 |
| | secretion | |
| | system | |
| 1008 | Type VI | MIC-36497 |
| | secretion | |
| | system | |
| 1009 | Type VI | MIC-36497 |
| | secretion | |
| | system | |
| 1010 | Type VI | MIC-36497 |
| | secretion | |
| | system | |
| 1011 | Type VI | MIC-29285 |
| | secretion | |
| | system | |
| 1012 | Type VI | MIC-82689 |
| | secretion | |
| | system | |
| 1013 | Type VI | MIC-82689 |
| | secretion | |
| | system | |
| 1014 | Type VI | MIC-82689 |
| | secretion | |
| | system | |
| 1015 | Type VI | MIC-87588, MIC-86605, MIC-54642, |
| | secretion | MIC-61954, MIC-29662 |
| | system | |
| | putative | |
| | effector | |
| 1016 | Type VI | MIC-87588, MIC-86605, MIC-54642, |
| | secretion | MIC-61954, MIC-29662 |
| | system | |
| | putative | |
| | effector | |
| 1017 | Type VI | MIC-87588, MIC-86605, MIC-54642, |
| | secretion | MIC-61954, MIC-29662 |
| | system | |
| | putative | |
| | effector | |
| 1018 | Type VI | MIC-70076, MIC-73019, MIC-73547 |
| | secretion | |
| | system | |
| | putative | |
| | effector | |
| 1019 | Type VI | MIC-70076, MIC-73019, MIC-73547 |
| | secretion | |
| | system | |
| | putative | |
| | effector | |
| 1020 | Type VI | MIC-70076, MIC-73019, MIC-73547 |
| | secretion | |
| | system | |
| | putative | |
| | effector | |
| 1021 | Type VI | MIC-70076, MIC-73019, MIC-73547 |
| | secretion | |
| | system | |
| | putative | |
| | effector | |
| 1022 | Type VI | MIC-70076, MIC-73019, MIC-73547 |
| | secretion | |
| | system | |
| | putative | |
| | effector | |
| 1023 | Type VI | MIC-52924 |
| | secretion | |
| | system | |
| | putative | |
| | effector | |
| 1024 | Type VI | MIC-61954, MIC-29662 |
| | secretion | |
| | system | |
| | putative | |
| | effector | |
| 1025 | Type VI | MIC-68773 |
| | secretion | |
| | system | |
| | putative | |
| | effector | |
| 1026 | Type VI | MIC-68773 |
| | secretion | |
| | system | |
| | putative | |
| | effector | |
| 1027 | Type VI | MIC-68773 |
| | secretion | |
| | system | |
| | putative | |
| | effector | |
| 1028 | Type VI | MIC-68773 |
| | secretion | |
| | system | |
| | putative | |
| | effector | |
| 1029 | Type VI | MIC-54778 |
| | secretion | |
| | system | |
| | putative | |
| | effector | |
| 1030 | Type VI | MIC-87198 |
| | secretion | |
| | system | |
| | putative | |
| | effector | |
| 1031 | Type VI | MIC-87198 |
| | secretion | |
| | system | |
| | putative | |
| | effector | |
| 1032 | Type VI | MIC-87198 |
| | secretion | |
| | system | |
| | putative | |
| | effector | |
| 1033 | Type VI | MIC-87198 |
| | secretion | |
| | system | |
| | putative | |
| | effector | |
| 1034 | Type VI | MIC-90405 |
| | secretion | |
| | system | |
| | putative | |
| | effector | |
| 1035 | Type VI | MIC-90405 |
| | secretion | |
| | system | |
| | putative | |
| | effector | |
| 1036 | Type VI | MIC-90405 |
| | secretion | |
| | system | |
| | putative | |
| | effector | |
| 1037 | Type VI | MIC-90405 |
| | secretion | |
| | system | |
| | putative | |
| | effector | |
| 1038 | Type VI | MIC-14439 |
| | secretion | |
| | system | |
| | putative | |
| | effector | |
| 1039 | Type VI | MIC-14439 |
| | secretion | |
| | system | |
| | putative | |
| | effector | |
| 1040 | Type VI | MIC-14439 |
| | secretion | |
| | system | |
| | putative | |
| | effector | |
| 1041 | Type VI | MIC-14439 |
| | secretion | |
| | system | |
| | putative | |
| | effector | |
| 1042 | Type VI | MIC-54778 |
| | secretion | |
| | system | |
| | putative | |
| | effector | |
| 1043 | Type VI | MIC-54778 |
| | secretion | |
| | system | |
| | putative | |
| | effector | |
| 1044 | Type VI | MIC-54778 |
| | secretion | |
| | system | |
| | putative | |
| | effector | |
| 1045 | Type VI | MIC-80455 |
| | secretion | |
| | system | |
| | putative | |
| | effector | |
| 1046 | Type VI | MIC-80455 |
| | secretion | |
| | system | |
| | putative | |
| | effector | |
| 1047 | Type VI | MIC-80455 |
| | secretion | |
| | system | |
| | putative | |
| | effector | |
| 1048 | Type VI | MIC-80455 |
| | secretion | |
| | system | |
| | putative | |
| | effector | |
| 1049 | Type VI | MIC-87588, MIC-86605, MIC-54642 |
| | secretion | |
| | system | |
| | putative | |
| | effector | |
| 1050 | Type VI | MIC-94504 |
| | secretion | |
| | system | |
| | putative | |
| | effector | |
| 1051 | Type VI | MIC-94504 |
| | secretion | |
| | system | |
| | putative | |
| | effector | |
| 1052 | Type VI | MIC-94504 |
| | secretion | |
| | system | |
| | putative | |
| | effector | |
| 1053 | Type VI | MIC-94504 |
| | secretion | |
| | system | |
| | putative | |
| | effector | |
| 1054 | Type VI | MIC-24837, MIC-55579 |
| | secretion | |
| | system | |
| | putative | |
| | effector | |
| 1055 | Type VI | MIC-24837, MIC-55579 |
| | secretion | |
| | system | |
| | putative | |
| | effector | |
| 1056 | Type VI | MIC-24837, MIC-55579 |
| | secretion | |
| | system | |
| | putative | |
| | effector | |
| 1057 | Type VI | MIC-24837, MIC-55579 |
| | secretion | |
| | system | |
| | putative | |
| | effector | |
| 1058 | Type VI | MIC-84492 |
| | secretion | |
| | system | |
| | putative | |
| | effector | |
| 1059 | Type VI | MIC-50391, MIC-69701 |
| | secretion | |
| | system | |
| | putative | |
| | effector | |
| 1060 | Type VI | MIC-50391, MIC-69701, MIC-52924 |
| | secretion | |
| | system | |
| | putative | |
| | effector | |
| 1061 | Type VI | MIC-50391, MIC-69701, MIC-52924 |
| | secretion | |
| | system | |
| | putative | |
| | effector | |
| 1062 | Type VI | MIC-50391, MIC-69701, MIC-52924 |
| | secretion | |
| | system | |
| | putative | |
| | effector | |
| 1063 | Type VI | MIC-75437, MIC-20446 |
| | secretion | |
| | system | |
| | putative | |
| | effector | |
| 1064 | Type VI | MIC-38993 |
| | secretion | |
| | system | |
| | putative | |
| | effector | |
| 1065 | Type VI | MIC-38993 |
| | secretion | |
| | system | |
| | putative | |
| | effector | |
| 1066 | Type VI | MIC-38993 |
| | secretion | |
| | system | |
| | putative | |
| | effector | |
| 1067 | Type VI | MIC-38993 |
| | secretion | |
| | system | |
| | putative | |
| | effector | |
| 1068 | Type VI | MIC-94458, MIC-30352, MIC-82867 |
| | secretion | |
| | system | |
| | putative | |
| | effector | |
| 1069 | Type VI | MIC-94458, MIC-30352, MIC-82867 |
| | secretion | |
| | system | |
| | putative | |
| | effector | |
| 1070 | Type VI | MIC-94458, MIC-30352, MIC-82867 |
| | secretion | |
| | system | |
| | putative | |
| | effector | |
| 1071 | Type VI | MIC-94458, MIC-30352, MIC-82867 |
| | secretion | |
| | system | |
| | putative | |
| | effector | |
| 1072 | Type VI | MIC-94458, MIC-30352, MIC-82867 |
| | secretion | |
| | system | |
| | putative | |
| | effector | |
| 1073 | Type VI | MIC-46385, MIC-62164 |
| | secretion | |
| | system | |
| | putative | |
| | effector | |
| 1074 | Type VI | MIC-46385, MIC-62164, MIC-87894 |
| | secretion | |
| | system | |
| | putative | |
| | effector | |
| 1075 | Type VI | MIC-46385, MIC-62164, MIC-87894 |
| | secretion | |
| | system | |
| | putative | |
| | effector | |
| 1076 | Type VI | MIC-46385, MIC-62164, MIC-87894 |
| | secretion | |
| | system | |
| | putative | |
| | effector | |
| 1077 | Type VI | MIC-46385, MIC-62164, MIC-87894 |
| | secretion | |
| | system | |
| | putative | |
| | effector | |
| 1078 | Type VI | MIC-14970, MIC-11290 |
| | secretion | |
| | system | |
| | putative | |
| | effector | |
| 1079 | Type VI | MIC-14970, MIC-11290 |
| | secretion | |
| | system | |
| | putative | |
| | effector | |
| 1080 | Type VI | MIC-14970, MIC-11290 |
| | secretion | |
| | system | |
| | putative | |
| | effector | |
| 1081 | Type VI | MIC-14970, MIC-11290 |
| | secretion | |
| | system | |
| | putative | |
| | effector | |
| 1082 | Type VI | MIC-36497 |
| | secretion | |
| | system | |
| | putative | |
| | effector | |
| 1083 | Type VI | MIC-36497 |
| | secretion | |
| | system | |
| | putative | |
| | effector | |
| 1084 | Type VI | MIC-36497 |
| | secretion | |
| | system | |
| | putative | |
| | effector | |
| 1085 | Type VI | MIC-36497 |
| | secretion | |
| | system | |
| | putative | |
| | effector | |
| 1086 | Type VI | MIC-82689 |
| | secretion | |
| | system | |
| | putative | |
| | effector | |
| 1087 | Type VI | MIC-82689 |
| | secretion | |
| | system | |
| | putative | |
| | effector | |
| 1088 | Type VI | MIC-82689 |
| | secretion | |
| | system | |
| | putative | |
| | effector | |
| 1089 | Type VI | MIC-82689 |
| | secretion | |
| | system | |
| | putative | |
| | effector | |

TABLE 2B
Protein sequences of endophytes

| SEQ ID | Sequence Description | Endophytes |
|---|---|---|
| 1090 | Flagellin | MIC-70076 |
| 1091 | Flagellin | MIC-70076 |
| 1092 | Flagellin | MIC-70076 |
| 1093 | Flagellin | MIC-70076 |
| 1094 | Flagellin | MIC-70076 |
| 1095 | Flagellin | MIC-70076 |
| 1096 | Flagellin | MIC-70076 |
| 1097 | Flagellin | MIC-73019, MIC-87588, MIC-86605, |
| | | MIC-54642, MIC-94504, MIC-24837, |
| | | MIC-55579, MIC-61954, MIC-29662, |
| | | MIC-38993, MIC-73547, MIC-14970, |
| | | MIC-11290, MIC-36497, MIC-82689 |
| 1098 | Flagellin | MIC-83010, MIC-68773, MIC-87588, |
| | | MIC-86605, MIC-54642, MIC-94504, |
| | | MIC-24837, MIC-55579, MIC-87198, |
| | | MIC-61954, MIC-29662, MIC-90405, |
| | | MIC-75437, MIC-38993, MIC-20446, |
| | | MIC-14970, MIC-11290, MIC-36497 |
| 1099 | Flagellin | MIC-68773, MIC-80455, MIC-87588, |
| | | MIC-86605, MIC-54642, MIC-94504, |
| | | MIC-24837, MIC-55579, MIC-87198, |
| | | MIC-61954, MIC-29662, MIC-90405, |
| | | MIC-38993, MIC-14970, MIC-11290, |
| | | MIC-36497, MIC-82689 |
| 1100 | Flagellin | MIC-68773, MIC-87588, MIC-86605, |
| | | MIC-54642, MIC-94504, MIC-24837, |
| | | MIC-55579, MIC-87198, MIC-61954, |
| | | MIC-29662, MIC-90405, MIC-38993, |
| | | MIC-14970, MIC-11290, MIC-36497 |
| 1101 | Flagellin | MIC-68901, MIC-81265, MIC-88834, |
| | | MIC-46385, MIC-87894 |
| 1102 | Flagellin | MIC-68773, MIC-86605, MIC-54642, |
| | | MIC-61954, MIC-29662, MIC-11290 |
| 1103 | Flagellin | MIC-70076, MIC-73019, MIC-14439, |
| | | MIC-73547 |
| 1104 | Flagellin | MIC-68773, MIC-86605, MIC-61954, |
| | | MIC-29662, MIC-11290 |
| 1105 | Flagellin | MIC-70076, MIC-73019, MIC-54778, |
| | | MIC-73547 |
| 1106 | Flagellin | MIC-70076, MIC-73019, MIC-73547 |
| 1107 | Flagellin | MIC-81265 |
| 1108 | Flagellin | MIC-61954 |
| 1109 | Flagellin | MIC-61954 |
| 1110 | Flagellin | MIC-61954 |
| 1111 | Flagellin | MIC-61954 |
| 1112 | Flagellin | MIC-68901, MIC-46385, MIC-87894 |
| 1113 | Flagellin | MIC-52924 |
| 1114 | Flagellin | MIC-68901 |
| 1115 | Flagellin | MIC-68773, MIC-54642, MIC-11290 |
| 1116 | Flagellin | MIC-73019, MIC-73547 |
| 1117 | Flagellin | MIC-73019, MIC-73547 |
| 1118 | Flagellin | MIC-73019, MIC-73547 |
| 1119 | Flagellin | MIC-82330 |
| 1120 | Flagellin | MIC-82330 |
| 1121 | Flagellin | MIC-82330 |
| 1122 | Flagellin | MIC-82330 |
| 1123 | Flagellin | MIC-82330, MIC-83740 |
| 1124 | Flagellin | MIC-82330, MIC-84492 |
| 1125 | Flagellin | MIC-82330, MIC-84492 |
| 1126 | Flagellin | MIC-68773 |
| 1127 | Flagellin | MIC-68773 |
| 1128 | Flagellin | MIC-68773 |
| 1129 | Flagellin | MIC-54778 |
| 1130 | Flagellin | MIC-54778 |
| 1131 | Flagellin | MIC-54778 |
| 1132 | Flagellin | MIC-54778 |
| 1133 | Flagellin | MIC-19814 |
| 1134 | Flagellin | MIC-19814 |
| 1135 | Flagellin | MIC-87588 |
| 1136 | Flagellin | MIC-87588 |
| 1137 | Flagellin | MIC-24837 |
| 1138 | Flagellin | MIC-24837 |
| 1139 | Flagellin | MIC-87198 |
| 1140 | Flagellin | MIC-87198 |
| 1141 | Flagellin | MIC-87198 |
| 1142 | Flagellin | MIC-87198 |
| 1143 | Flagellin | MIC-90405 |
| 1144 | Flagellin | MIC-90405 |
| 1145 | Flagellin | MIC-90405 |
| 1146 | Flagellin | MIC-90405 |
| 1147 | Flagellin | MIC-90405 |
| 1148 | Flagellin | MIC-14970 |
| 1149 | Flagellin | MIC-68901 |
| 1150 | Flagellin | MIC-68901 |
| 1151 | Flagellin | MIC-88834 |
| 1152 | Flagellin | MIC-88834 |
| 1153 | Flagellin | MIC-14439 |
| 1154 | Flagellin | MIC-14439 |
| 1155 | Flagellin | MIC-14439 |
| 1156 | Flagellin | MIC-54642 |
| 1157 | Flagellin | MIC-29662 |
| 1158 | Flagellin | MIC-79613 |
| 1159 | Flagellin | MIC-79613 |
| 1160 | Flagellin | MIC-79613 |
| 1161 | Flagellin | MIC-79613 |
| 1162 | Flagellin | MIC-79613, MIC-53518 |
| 1163 | Flagellin | MIC-79613, MIC-53518 |
| 1164 | Flagellin | MIC-79613, MIC-53518 |
| 1165 | Flagellin | MIC-53518 |
| 1166 | Flagellin | MIC-53518 |
| 1167 | Flagellin | MIC-53518 |
| 1168 | Flagellin | MIC-53518 |
| 1169 | Flagellin | MIC-82330 |
| 1170 | Flagellin | MIC-83010 |
| 1171 | Flagellin | MIC-83010 |
| 1172 | Flagellin | MIC-83010, MIC-75437 |
| 1173 | Flagellin | MIC-83010, MIC-75437, MIC-20446 |
| 1174 | Flagellin | MIC-83740 |
| 1175 | Flagellin | MIC-83740 |
| 1176 | Flagellin | MIC-83740 |
| 1177 | Flagellin | MIC-83740 |
| 1178 | Flagellin | MIC-83740, MIC-36254, MIC-29285 |
| 1179 | Flagellin | MIC-83740, MIC-87084 |
| 1180 | Flagellin | MIC-83740, MIC-29285 |
| 1181 | Flagellin | MIC-80455 |
| 1182 | Flagellin | MIC-80455 |
| 1183 | Flagellin | MIC-80455 |
| 1184 | Flagellin | MIC-80455 |
| 1185 | Flagellin | MIC-80455 |
| 1186 | Flagellin | MIC-80455 |
| 1187 | Flagellin | MIC-86605 |
| 1188 | Flagellin | MIC-86605 |
| 1189 | Flagellin | MIC-86605, MIC-82689 |
| 1190 | Flagellin | MIC-94504 |
| 1191 | Flagellin | MIC-94504 |
| 1192 | Flagellin | MIC-94504, MIC-14970 |
| 1193 | Flagellin | MIC-24837, MIC-55579 |
| 1194 | Flagellin | MIC-55579 |
| 1195 | Flagellin | MIC-55579 |
| 1196 | Flagellin | MIC-55579 |
| 1197 | Flagellin | MIC-55579 |
| 1198 | Flagellin | MIC-19845 |
| 1199 | Flagellin | MIC-19845 |
| 1200 | Flagellin | MIC-19845 |
| 1201 | Flagellin | MIC-19845 |
| 1202 | Flagellin | MIC-19845 |
| 1203 | Flagellin | MIC-19845, MIC-85267 |
| 1204 | Flagellin | MIC-19845, MIC-14439, MIC-82689 |
| 1205 | Flagellin | MIC-84492 |
| 1206 | Flagellin | MIC-84492 |
| 1207 | Flagellin | MIC-84492 |
| 1208 | Flagellin | MIC-84492 |
| 1209 | Flagellin | MIC-84492 |
| 1210 | Flagellin | MIC-84492, MIC-36254, MIC-29285 |
| 1211 | Flagellin | MIC-50391, MIC-69701, MIC-52924 |
| 1212 | Flagellin | MIC-50391, MIC-69701, MIC-52924 |
| 1213 | Flagellin | MIC-50391, MIC-69701, MIC-52924 |
| 1214 | Flagellin | MIC-50391, MIC-69701, MIC-52924, |
| | | MIC-82689 |
| 1215 | Flagellin | MIC-85267 |
| 1216 | Flagellin | MIC-85267 |
| 1217 | Flagellin | MIC-85267 |
| 1218 | Flagellin | MIC-85267 |
| 1219 | Flagellin | MIC-85267 |
| 1220 | Flagellin | MIC-85267 |
| 1221 | Flagellin | MIC-85267, MIC-36254 |
| 1222 | Flagellin | MIC-90405, MIC-38993 |
| 1223 | Flagellin | MIC-75437 |
| 1224 | Flagellin | MIC-75437 |
| 1225 | Flagellin | MIC-75437, MIC-20446 |
| 1226 | Flagellin | MIC-38993 |
| 1227 | Flagellin | MIC-38993 |
| 1228 | Flagellin | MIC-20446 |
| 1229 | Flagellin | MIC-20446 |
| 1230 | Flagellin | MIC-94135 |
| 1231 | Flagellin | MIC-94135 |
| 1232 | Flagellin | MIC-94135 |
| 1233 | Flagellin | MIC-94458 |
| 1234 | Flagellin | MIC-94458 |
| 1235 | Flagellin | MIC-94458 |
| 1236 | Flagellin | MIC-94458, MIC-30352, MIC-82867 |
| 1237 | Flagellin | MIC-46385, MIC-62164, MIC-87894 |
| 1238 | Flagellin | MIC-46385, MIC-87894 |
| 1239 | Flagellin | MIC-46385, MIC-87894 |
| 1240 | Flagellin | MIC-62164 |
| 1241 | Flagellin | MIC-62164 |
| 1242 | Flagellin | MIC-30352 |
| 1243 | Flagellin | MIC-82867 |
| 1244 | Flagellin | MIC-82867 |
| 1245 | Flagellin | MIC-82867 |
| 1246 | Flagellin | MIC-82867 |
| 1247 | Flagellin | MIC-36254 |
| 1248 | Flagellin | MIC-36254 |
| 1249 | Flagellin | MIC-36254 |
| 1250 | Flagellin | MIC-36254 |
| 1251 | Flagellin | MIC-36254 |
| 1252 | Flagellin | MIC-36254 |
| 1253 | Flagellin | MIC-73547 |
| 1254 | Flagellin | MIC-73547 |
| 1255 | Flagellin | MIC-73547 |
| 1256 | Flagellin | MIC-11290 |
| 1257 | Flagellin | MIC-87084 |
| 1258 | Flagellin | MIC-87084 |
| 1259 | Flagellin | MIC-87084 |
| 1260 | Flagellin | MIC-87084 |
| 1261 | Flagellin | MIC-87084 |
| 1262 | Flagellin | MIC-36497 |
| 1263 | Flagellin | MIC-36497 |
| 1264 | Flagellin | MIC-36497 |
| 1265 | Flagellin | MIC-36497, MIC-82689 |
| 1266 | Flagellin | MIC-29285 |
| 1267 | Flagellin | MIC-29285 |
| 1268 | Flagellin | MIC-29285 |
| 1269 | Flagellin | MIC-29285 |
| 1270 | Flagellin | MIC-82689 |
| 1271 | Flagellin | MIC-82689 |
| 1272 | Flagellin | MIC-82689 |
| 1273 | O-Antigen | MIC-70076, MIC-73019, MIC-73547 |
| | biosynthesis | |
| 1274 | O-Antigen | MIC-70076, MIC-73019, MIC-73547 |
| | biosynthesis | |
| 1275 | O-Antigen | MIC-70076, MIC-73019, MIC-73547 |
| | biosynthesis | |
| 1276 | O-Antigen | MIC-70076, MIC-73019, MIC-73547 |
| | biosynthesis | |
| 1277 | O-Antigen | MIC-19845 |
| | biosynthesis | |
| 1278 | O-Antigen | MIC-85267 |
| | biosynthesis | |
| 1279 | O-Antigen | MIC-38993 |
| | biosynthesis | |
| 1280 | O-Antigen | MIC-38993 |
| | biosynthesis | |
| 1281 | O-Antigen | MIC-38993, MIC-82689 |
| | biosynthesis | |
| 1282 | O-Antigen | MIC-38993, MIC-82689 |
| | biosynthesis | |
| 1283 | O-Antigen | MIC-36497 |
| | biosynthesis | |
| 1284 | O-Antigen | MIC-82689 |
| | biosynthesis | |
| 1285 | O-Antigen | MIC-82689 |
| | biosynthesis | |
| 1286 | pseudaminic | MIC-80455, MIC-87588, MIC-86605, |
| | acid biosynthesis | MIC-54642, MIC-24837, MIC-55579, |
| | | MIC-61954, MIC-29662, MIC-90405, |
| | | MIC-14970, MIC-11290 |
| 1287 | pseudaminic | MIC-87588, MIC-86605, MIC-54642, |
| | acid biosynthesis | MIC-24837, MIC-55579, MIC-61954, |
| | | MIC-29662, MIC-90405, MIC-14970, |
| | | MIC-11290 |
| 1288 | pseudaminic | MIC-70076, MIC-73019, MIC-73547 |
| | acid biosynthesis | |
| 1289 | pseudaminic | MIC-70076, MIC-73019, MIC-73547 |
| | acid biosynthesis | |
| 1290 | pseudaminic | MIC-70076, MIC-73019, MIC-73547 |
| | acid biosynthesis | |
| 1291 | pseudaminic | MIC-70076, MIC-73019, MIC-73547 |
| | acid biosynthesis | |
| 1292 | pseudaminic | MIC-70076, MIC-73019, MIC-73547 |
| | acid biosynthesis | |
| 1293 | pseudaminic | MIC-70076, MIC-73019, MIC-73547 |
| | acid biosynthesis | |
| 1294 | pseudaminic | MIC-70076, MIC-73019, MIC-73547 |
| | acid biosynthesis | |
| 1295 | pseudaminic | MIC-79613, MIC-53518 |
| | acid biosynthesis | |
| 1296 | pseudaminic | MIC-79613, MIC-53518 |
| | acid biosynthesis | |
| 1297 | pseudaminic | MIC-80455 |
| | acid biosynthesis | |
| 1298 | pseudaminic | MIC-19845 |
| | acid biosynthesis | |
| 1299 | pseudaminic | MIC-19845 |
| | acid biosynthesis | |
| 1300 | pseudaminic | MIC-19845 |
| | acid biosynthesis | |
| 1301 | pseudaminic | MIC-19845 |
| | acid biosynthesis | |
| 1302 | pseudaminic | MIC-50391, MIC-69701, MIC-52924 |
| | acid biosynthesis | |
| 1303 | pseudaminic | MIC-85267 |
| | acid biosynthesis | |
| 1304 | pseudaminic | MIC-38993 |
| | acid biosynthesis | |
| 1305 | pseudaminic | MIC-38993 |
| | acid biosynthesis | |
| 1306 | pseudaminic | MIC-38993 |
| | acid biosynthesis | |
| 1307 | pseudaminic | MIC-38993 |
| | acid biosynthesis | |
| 1308 | pseudaminic | MIC-38993 |
| | acid biosynthesis | |
| 1309 | pseudaminic | MIC-38993 |
| | acid biosynthesis | |
| 1310 | pseudaminic | MIC-38993, MIC-82689 |
| | acid biosynthesis | |
| 1311 | pseudaminic | MIC-94458, MIC-30352, MIC-82867 |
| | acid biosynthesis | |
| 1312 | pseudaminic | MIC-82689 |
| | acid biosynthesis | |
| 1313 | pseudaminic | MIC-82689 |
| | acid biosynthesis | |
| 1314 | pseudaminic | MIC-82689 |
| | acid biosynthesis | |
| 1315 | pseudaminic | MIC-82689 |
| | acid biosynthesis | |
| 1316 | pseudaminic | MIC-82689 |
| | acid biosynthesis | |
| 1317 | pseudaminic | MIC-82689 |
| | acid biosynthesis | |
| 1318 | Tailocin gene | MIC-70076, MIC-73019, MIC-87588, |
| | cluster | MIC-86605, MIC-54642, MIC-73547 |
| 1319 | Tailocin gene | MIC-87588, MIC-86605, MIC-54642, |
| | cluster | MIC-61954, MIC-29662 |
| 1320 | Tailocin gene | MIC-70076, MIC-73019, MIC-24837, |
| | cluster | MIC-55579, MIC-73547 |
| 1321 | Tailocin gene | MIC-70076, MIC-73019, MIC-24837, |
| | cluster | MIC-55579, MIC-73547 |
| 1322 | Tailocin gene | MIC-70076, MIC-73019, MIC-50391, |
| | cluster | MIC-69701, MIC-73547 |
| 1323 | Tailocin gene | MIC-70076, MIC-73019, MIC-38993, |
| | cluster | MIC-73547 |
| 1324 | Tailocin gene | MIC-70076, MIC-73019, MIC-38993, |
| | cluster | MIC-73547 |
| 1325 | Tailocin gene | MIC-70076, MIC-73019, MIC-38993, |
| | cluster | MIC-73547 |
| 1326 | Tailocin gene | MIC-70076, MIC-73019, MIC-73547 |
| | cluster | |
| 1327 | Tailocin gene | MIC-70076, MIC-73019, MIC-73547 |
| | cluster | |
| 1328 | Tailocin gene | MIC-70076, MIC-73019, MIC-73547 |
| | cluster | |
| 1329 | Tailocin gene | MIC-70076, MIC-73019, MIC-73547 |
| | cluster | |
| 1330 | Tailocin gene | MIC-70076, MIC-73019, MIC-73547 |
| | cluster | |
| 1331 | Tailocin gene | MIC-70076, MIC-73019, MIC-73547 |
| | cluster | |
| 1332 | Tailocin gene | MIC-70076, MIC-73019, MIC-73547 |
| | cluster | |
| 1333 | Tailocin gene | MIC-70076, MIC-73019, MIC-73547 |
| | cluster | |
| 1334 | Tailocin gene | MIC-70076, MIC-73019, MIC-73547 |
| | cluster | |
| 1335 | Tailocin gene | MIC-70076, MIC-73019, MIC-73547 |
| | cluster | |
| 1336 | Tailocin gene | MIC-70076, MIC-73019, MIC-73547 |
| | cluster | |
| 1337 | Tailocin gene | MIC-70076, MIC-73019, MIC-73547 |
| | cluster | |
| 1338 | Tailocin gene | MIC-70076, MIC-73019, MIC-73547 |
| | cluster | |
| 1339 | Tailocin gene | MIC-70076, MIC-73019, MIC-73547 |
| | cluster | |
| 1340 | Tailocin gene | MIC-70076, MIC-73019, MIC-73547 |
| | cluster | |
| 1341 | Tailocin gene | MIC-70076, MIC-73019, MIC-73547 |
| | cluster | |
| 1342 | Tailocin gene | MIC-70076, MIC-73019, MIC-73547 |
| | cluster | |
| 1343 | Tailocin gene | MIC-70076, MIC-73019, MIC-73547 |
| | cluster | |
| 1344 | Tailocin gene | MIC-70076, MIC-73019, MIC-73547 |
| | cluster | |
| 1345 | Tailocin gene | MIC-70076, MIC-73019, MIC-73547 |
| | cluster | |
| 1346 | Tailocin gene | MIC-70076, MIC-73019, MIC-73547 |
| | cluster | |
| 1347 | Tailocin gene | MIC-70076, MIC-73019, MIC-73547 |
| | cluster | |
| 1348 | Tailocin gene | MIC-70076, MIC-73019, MIC-73547 |
| | cluster | |
| 1349 | Tailocin gene | MIC-70076, MIC-73019, MIC-73547 |
| | cluster | |
| 1350 | Tailocin gene | MIC-70076, MIC-73019, MIC-73547 |
| | cluster | |
| 1351 | Tailocin gene | MIC-70076, MIC-73019, MIC-73547, |
| | cluster | MIC-14970, MIC-11290 |
| 1352 | Tailocin gene | MIC-70076, MIC-73019, MIC-38993, |
| | cluster | MIC-73547 |
| 1353 | Tailocin gene | MIC-70076, MIC-73019, MIC-94458, |
| | cluster | MIC-30352, MIC-82867, MIC-73547 |
| 1354 | Tailocin gene | MIC-70076, MIC-73019, MIC-73547 |
| | cluster | |
| 1355 | Tailocin gene | MIC-70076, MIC-73019, MIC-73547 |
| | cluster | |
| 1356 | Tailocin gene | MIC-70076, MIC-73019, MIC-73547 |
| | cluster | |
| 1357 | Tailocin gene | MIC-70076, MIC-73019, MIC-73547 |
| | cluster | |
| 1358 | Tailocin gene | MIC-70076, MIC-73019, MIC-73547 |
| | cluster | |
| 1359 | Tailocin gene | MIC-70076, MIC-73019, MIC-73547 |
| | cluster | |
| 1360 | Tailocin gene | MIC-70076, MIC-73019, MIC-73547 |
| | cluster | |
| 1361 | Tailocin gene | MIC-70076, MIC-73019, MIC-73547 |
| | cluster | |
| 1362 | Tailocin gene | MIC-70076, MIC-73019, MIC-73547 |
| | cluster | |
| 1363 | Tailocin gene | MIC-70076, MIC-73019, MIC-73547 |
| | cluster | |
| 1364 | Tailocin gene | MIC-70076, MIC-73019, MIC-73547 |
| | cluster | |
| 1365 | Tailocin gene | MIC-70076, MIC-73019, MIC-73547 |
| | cluster | |
| 1366 | Tailocin gene | MIC-70076, MIC-73019, MIC-73547 |
| | cluster | |
| 1367 | Tailocin gene | MIC-70076, MIC-73019, MIC-73547 |
| | cluster | |
| 1368 | Tailocin gene | MIC-70076, MIC-73019, MIC-73547 |
| | cluster | |
| 1369 | Tailocin gene | MIC-70076, MIC-73019, MIC-73547 |
| | cluster | |
| 1370 | Tailocin gene | MIC-70076, MIC-73019, MIC-73547 |
| | cluster | |
| 1371 | Tailocin gene | MIC-70076, MIC-73019, MIC-73547 |
| | cluster | |
| 1372 | Tailocin gene | MIC-70076, MIC-73019, MIC-73547 |
| | cluster | |
| 1373 | Tailocin gene | MIC-70076, MIC-73019, MIC-73547 |
| | cluster | |
| 1374 | Tailocin gene | MIC-82330, MIC-61954, MIC-29662, |
| | cluster | MIC-36254 |
| 1375 | Tailocin gene | MIC-68901, MIC-19814 |
| | cluster | |
| 1376 | Tailocin gene | MIC-68901, MIC-19814 |
| | cluster | |
| 1377 | Tailocin gene | MIC-68901, MIC-19814 |
| | cluster | |
| 1378 | Tailocin gene | MIC-68901, MIC-19814 |
| | cluster | |
| 1379 | Tailocin gene | MIC-68901, MIC-19814 |
| | cluster | |
| 1380 | Tailocin gene | MIC-68901, MIC-19814 |
| | cluster | |
| 1381 | Tailocin gene | MIC-68901, MIC-19814 |
| | cluster | |
| 1382 | Tailocin gene | MIC-68901, MIC-19814 |
| | cluster | |
| 1383 | Tailocin gene | MIC-68901, MIC-19814 |
| | cluster | |
| 1384 | Tailocin gene | MIC-68901, MIC-19814 |
| | cluster | |
| 1385 | Tailocin gene | MIC-68901, MIC-19814 |
| | cluster | |
| 1386 | Tailocin gene | MIC-68901, MIC-19814 |
| | cluster | |
| 1387 | Tailocin gene | MIC-68901, MIC-19814, MIC-87894, |
| | cluster | MIC-29285 |
| 1388 | Tailocin gene | MIC-87588, MIC-86605, MIC-54642, |
| | cluster | MIC-24837, MIC-55579, MIC-84492, |
| | | MIC-85267 |
| 1389 | Tailocin gene | MIC-87588, MIC-54642, MIC-90405 |
| | cluster | |
| 1390 | Tailocin gene | MIC-83010, MIC-87198, MIC-94458, |
| | | MIC-46385, MIC-62164, MIC-30352, |
| | cluster | MIC-82867, MIC-14970, MIC-11290, |
| | | MIC-87084 |
| 1391 | Tailocin gene | MIC-68901, MIC-19814 |
| | cluster | |
| 1392 | Tailocin gene | MIC-68901, MIC-19814 |
| | cluster | |
| 1393 | Tailocin gene | MIC-68901, MIC-19814 |
| | cluster | |
| 1394 | Tailocin gene | MIC-68901, MIC-19814 |
| | cluster | |
| 1395 | Tailocin gene | MIC-68901, MIC-19814 |
| | cluster | |
| 1396 | Tailocin gene | MIC-68901, MIC-19814 |
| | cluster | |
| 1397 | Tailocin gene | MIC-68901, MIC-19814 |
| | cluster | |
| 1398 | Tailocin gene | MIC-68901, MIC-19814 |
| | cluster | |
| 1399 | Tailocin gene | MIC-87588, MIC-86605, MIC-54642, |
| | cluster | MIC-24837, MIC-55579, MIC-94458, |
| | | MIC-30352, MIC-82867 |
| 1400 | Tailocin gene | MIC-24837, MIC-55579, MIC-90405 |
| | cluster | |
| 1401 | Tailocin gene | MIC-24837, MIC-55579, MIC-90405 |
| | cluster | |
| 1402 | Tailocin gene | MIC-68901 |
| | cluster | |
| 1403 | Tailocin gene | MIC-68901 |
| | cluster | |
| 1404 | Tailocin gene | MIC-68901 |
| | cluster | |
| 1405 | Tailocin gene | MIC-52924 |
| | cluster | |
| 1406 | Tailocin gene | MIC-52924 |
| | cluster | |
| 1407 | Tailocin gene | MIC-68901 |
| | cluster | |
| 1408 | Tailocin gene | MIC-52924 |
| | cluster | |
| 1409 | Tailocin gene | MIC-52924 |
| | cluster | |
| 1410 | Tailocin gene | MIC-83010, MIC-86605, MIC-81265, |
| | cluster | MIC-88834, MIC-84492, MIC-75437, |
| | | MIC-20446, MIC-94135, MIC-46385, |
| | | MIC-62164, MIC-87084, MIC-36497, |
| | | MIC-82689 |
| 1411 | Tailocin gene | MIC-83010, MIC-81265, MIC-88834, |
| | cluster | MIC-84492, MIC-75437, MIC-20446, |
| | | MIC-94135, MIC-46385, MIC-62164, |
| | | MIC-87084, MIC-36497, MIC-82689 |
| 1412 | Tailocin gene | MIC-81265, MIC-88834 |
| | cluster | |
| 1413 | Tailocin gene | MIC-81265, MIC-88834, MIC-84492, |
| | cluster | MIC-75437, MIC-20446 |
| 1414 | Tailocin gene | MIC-86605, MIC-81265, MIC-88834, |
| | cluster | MIC-84492, MIC-75437, MIC-20446, |
| | | MIC-94135, MIC-36497 |
| 1415 | Tailocin gene | MIC-86605, MIC-81265, MIC-88834, |
| | cluster | MIC-84492, MIC-75437, MIC-20446, |
| | | MIC-94135, MIC-36497, MIC-82689 |
| 1416 | Tailocin gene | MIC-86605, MIC-81265, MIC-88834, |
| | cluster | MIC-84492, MIC-75437, MIC-20446, |
| | | MIC-94135, MIC-36497, MIC-82689 |
| 1417 | Tailocin gene | MIC-83740, MIC-61954, MIC-29662 |
| | cluster | |
| 1418 | Tailocin gene | MIC-61954, MIC-29662 |
| | cluster | |
| 1419 | Tailocin gene | MIC-61954, MIC-29662 |
| | cluster | |
| 1420 | Tailocin gene | MIC-61954, MIC-29662 |
| | cluster | |
| 1421 | Tailocin gene | MIC-61954, MIC-29662 |
| | cluster | |
| 1422 | Tailocin gene | MIC-61954, MIC-29662 |
| | cluster | |
| 1423 | Tailocin gene | MIC-61954, MIC-29662 |
| | cluster | |
| 1424 | Tailocin gene | MIC-61954, MIC-29662 |
| | cluster | |
| 1425 | Tailocin gene | MIC-61954, MIC-29662 |
| | cluster | |
| 1426 | Tailocin gene | MIC-82330 |
| | cluster | |
| 1427 | Tailocin gene | MIC-68773 |
| | cluster | |
| 1428 | Tailocin gene | MIC-68773 |
| | cluster | |
| 1429 | Tailocin gene | MIC-68773 |
| | cluster | |
| 1430 | Tailocin gene | MIC-68773 |
| | cluster | |
| 1431 | Tailocin gene | MIC-68773 |
| | cluster | |
| 1432 | Tailocin gene | MIC-68773 |
| | cluster | |
| 1433 | Tailocin gene | MIC-68773 |
| | cluster | |
| 1434 | Tailocin gene | MIC-54778 |
| | cluster | |
| 1435 | Tailocin gene | MIC-19814 |
| | cluster | |
| 1436 | Tailocin gene | MIC-19814 |
| | cluster | |
| 1437 | Tailocin gene | MIC-19814 |
| | cluster | |
| 1438 | Tailocin gene | MIC-19814 |
| | cluster | |
| 1439 | Tailocin gene | MIC-19814 |
| | cluster | |
| 1440 | Tailocin gene | MIC-19814 |
| | cluster | |
| 1441 | Tailocin gene | MIC-19814 |
| | cluster | |
| 1442 | Tailocin gene | MIC-19814 |
| | cluster | |
| 1443 | Tailocin gene | MIC-87198 |
| | cluster | |
| 1444 | Tailocin gene | MIC-87198 |
| | cluster | |
| 1445 | Tailocin gene | MIC-87198 |
| | cluster | |
| 1446 | Tailocin gene | MIC-87198 |
| | cluster | |
| 1447 | Tailocin gene | MIC-87198 |
| | cluster | |
| 1448 | Tailocin gene | MIC-87198 |
| | cluster | |
| 1449 | Tailocin gene | MIC-90405 |
| | cluster | |
| 1450 | Tailocin gene | MIC-90405 |
| | cluster | |
| 1451 | Tailocin gene | MIC-90405 |
| | cluster | |
| 1452 | Tailocin gene | MIC-90405 |
| | cluster | |
| 1453 | Tailocin gene | MIC-90405 |
| | cluster | |
| 1454 | Tailocin gene | MIC-90405 |
| | cluster | |
| 1455 | Tailocin gene | MIC-90405 |
| | cluster | |
| 1456 | Tailocin gene | MIC-90405 |
| | cluster | |
| 1457 | Tailocin gene | MIC-90405 |
| | cluster | |
| 1458 | Tailocin gene | MIC-90405 |
| | cluster | |
| 1459 | Tailocin gene | MIC-90405 |
| | cluster | |
| 1460 | Tailocin gene | MIC-87894 |
| | cluster | |
| 1461 | Tailocin gene | MIC-87894 |
| | cluster | |
| 1462 | Tailocin gene | MIC-87894 |
| | cluster | |
| 1463 | Tailocin gene | MIC-87894 |
| | cluster | |
| 1464 | Tailocin gene | MIC-87894 |
| | cluster | |
| 1465 | Tailocin gene | MIC-87894 |
| | cluster | |
| 1466 | Tailocin gene | MIC-87894 |
| | cluster | |
| 1467 | Tailocin gene | MIC-87894 |
| | cluster | |
| 1468 | Tailocin gene | MIC-87894 |
| | cluster | |
| 1469 | Tailocin gene | MIC-87894 |
| | cluster | |
| 1470 | Tailocin gene | MIC-87894 |
| | cluster | |
| 1471 | Tailocin gene | MIC-68901 |
| | cluster | |
| 1472 | Tailocin gene | MIC-14439 |
| | cluster | |
| 1473 | Tailocin gene | MIC-14439 |
| | cluster | |
| 1474 | Tailocin gene | MIC-14439 |
| | cluster | |
| 1475 | Tailocin gene | MIC-14439 |
| | cluster | |
| 1476 | Tailocin gene | MIC-14439 |
| | cluster | |
| 1477 | Tailocin gene | MIC-14439 |
| | cluster | |
| 1478 | Tailocin gene | MIC-68901 |
| | cluster | |
| 1479 | Tailocin gene | MIC-14439 |
| | cluster | |
| 1480 | Tailocin gene | MIC-14439 |
| | cluster | |
| 1481 | Tailocin gene | MIC-14439 |
| | cluster | |
| 1482 | Tailocin gene | MIC-14439 |
| | cluster | |
| 1483 | Tailocin gene | MIC-14439 |
| | cluster | |
| 1484 | Tailocin gene | MIC-87588, MIC-86605, MIC-54642 |
| | cluster | |
| 1485 | Tailocin gene | MIC-87588, MIC-86605, MIC-54642 |
| | cluster | |
| 1486 | Tailocin gene | MIC-87588, MIC-86605, MIC-54642 |
| | cluster | |
| 1487 | Tailocin gene | MIC-87588, MIC-86605, MIC-54642 |
| | cluster | |
| 1488 | Tailocin gene | MIC-87588, MIC-86605, MIC-54642 |
| | cluster | |
| 1489 | Tailocin gene | MIC-87588, MIC-86605, MIC-54642 |
| | cluster | |
| 1490 | Tailocin gene | MIC-87588, MIC-86605, MIC-54642 |
| | cluster | |
| 1491 | Tailocin gene | MIC-87588, MIC-86605, MIC-54642 |
| | cluster | |
| 1492 | Tailocin gene | MIC-87588, MIC-86605, MIC-54642 |
| | cluster | |
| 1493 | Tailocin gene | MIC-87588, MIC-86605, MIC-54642 |
| | cluster | |
| 1494 | Tailocin gene | MIC-87588, MIC-86605, MIC-54642 |
| | cluster | |
| 1495 | Tailocin gene | MIC-87588, MIC-86605, MIC-54642 |
| | cluster | |
| 1496 | Tailocin gene | MIC-87588, MIC-86605, MIC-54642 |
| | cluster | |
| 1497 | Tailocin gene | MIC-87588, MIC-86605, MIC-54642 |
| | cluster | |
| 1498 | Tailocin gene | MIC-87588, MIC-86605, MIC-54642, |
| | cluster | MIC-84492 |
| 1499 | Tailocin gene | MIC-87588, MIC-86605, MIC-54642, |
| | cluster | MIC-84492 |
| 1500 | Tailocin gene | MIC-87588, MIC-86605, MIC-54642, |
| | cluster | MIC-84492 |
| 1501 | Tailocin gene | MIC-87588, MIC-86605, MIC-54642, |
| | cluster | MIC-84492 |
| 1502 | Tailocin gene | MIC-87588, MIC-86605, MIC-54642, |
| | cluster | MIC-84492 |
| 1503 | Tailocin gene | MIC-87588, MIC-86605, MIC-54642, |
| | cluster | MIC-84492 |
| 1504 | Tailocin gene | MIC-87588, MIC-86605, MIC-54642, |
| | cluster | MIC-84492 |
| 1505 | Tailocin gene | MIC-87588, MIC-86605, MIC-54642, |
| | cluster | MIC-84492 |
| 1506 | Tailocin gene | MIC-87588, MIC-86605, MIC-54642, |
| | cluster | MIC-84492 |
| 1507 | Tailocin gene | MIC-87588, MIC-86605, MIC-54642, |
| | cluster | MIC-29285 |
| 1508 | Tailocin gene | MIC-87588, MIC-54642 |
| | cluster | |
| 1509 | Tailocin gene | MIC-86605, MIC-94504, MIC-75437, |
| | cluster | MIC-14439, MIC-20446, MIC-94135, |
| | | MIC-87084, MIC-36497, MIC-82689 |
| 1510 | Tailocin gene | MIC-79613 |
| | cluster | |
| 1511 | Tailocin gene | MIC-79613 |
| | cluster | |
| 1512 | Tailocin gene | MIC-79613 |
| | cluster | |
| 1513 | Tailocin gene | MIC-79613 |
| | cluster | |
| 1514 | Tailocin gene | MIC-79613 |
| | cluster | |
| 1515 | Tailocin gene | MIC-79613 |
| | cluster | |
| 1516 | Tailocin gene | MIC-79613, MIC-53518 |
| | cluster | |
| 1517 | Tailocin gene | MIC-79613, MIC-53518 |
| | cluster | |
| 1518 | Tailocin gene | MIC-79613, MIC-53518 |
| | cluster | |
| 1519 | Tailocin gene | MIC-79613, MIC-53518 |
| | cluster | |
| 1520 | Tailocin gene | MIC-79613, MIC-53518 |
| | cluster | |
| 1521 | Tailocin gene | MIC-79613, MIC-53518 |
| | cluster | |
| 1522 | Tailocin gene | MIC-79613, MIC-53518 |
| | cluster | |
| 1523 | Tailocin gene | MIC-79613, MIC-53518 |
| | cluster | |
| 1524 | Tailocin gene | MIC-79613, MIC-53518 |
| | cluster | |
| 1525 | Tailocin gene | MIC-79613, MIC-53518 |
| | cluster | |
| 1526 | Tailocin gene | MIC-79613, MIC-53518 |
| | cluster | |
| 1527 | Tailocin gene | MIC-79613, MIC-53518 |
| | cluster | |
| 1528 | Tailocin gene | MIC-79613, MIC-53518 |
| | cluster | |
| 1529 | Tailocin gene | MIC-79613, MIC-53518 |
| | cluster | |
| 1530 | Tailocin gene | MIC-79613, MIC-53518 |
| | cluster | |
| 1531 | Tailocin gene | MIC-79613, MIC-53518 |
| | cluster | |
| 1532 | Tailocin gene | MIC-79613, MIC-53518 |
| | cluster | |
| 1533 | Tailocin gene | MIC-79613, MIC-53518 |
| | cluster | |
| 1534 | Tailocin gene | MIC-79613, MIC-53518 |
| | cluster | |
| 1535 | Tailocin gene | MIC-79613, MIC-53518 |
| | cluster | |
| 1536 | Tailocin gene | MIC-79613, MIC-53518 |
| | cluster | |
| 1537 | Tailocin gene | MIC-79613, MIC-53518 |
| | cluster | |
| 1538 | Tailocin gene | MIC-79613, MIC-53518 |
| | cluster | |
| 1539 | Tailocin gene | MIC-79613, MIC-53518 |
| | cluster | |
| 1540 | Tailocin gene | MIC-79613, MIC-53518 |
| | cluster | |
| 1541 | Tailocin gene | MIC-79613, MIC-53518 |
| | cluster | |
| 1542 | Tailocin gene | MIC-79613, MIC-53518 |
| | cluster | |
| 1543 | Tailocin gene | MIC-79613, MIC-53518 |
| | cluster | |
| 1544 | Tailocin gene | MIC-79613, MIC-53518 |
| | cluster | |
| 1545 | Tailocin gene | MIC-79613, MIC-53518, MIC-38993 |
| | cluster | |
| 1546 | Tailocin gene | MIC-79613, MIC-53518, MIC-38993 |
| | cluster | |
| 1547 | Tailocin gene | MIC-79613, MIC-53518, MIC-29285 |
| | cluster | |
| 1548 | Tailocin gene | MIC-53518 |
| | cluster | |
| 1549 | Tailocin gene | MIC-53518 |
| | cluster | |
| 1550 | Tailocin gene | MIC-53518 |
| | cluster | |
| 1551 | Tailocin gene | MIC-53518 |
| | cluster | |
| 1552 | Tailocin gene | MIC-53518 |
| | cluster | |
| 1553 | Tailocin gene | MIC-53518 |
| | cluster | |
| 1554 | Tailocin gene | MIC-53518 |
| | cluster | |
| 1555 | Tailocin gene | MIC-83010, MIC-46385, MIC-62164 |
| | cluster | |
| 1556 | Tailocin gene | MIC-83010, MIC-46385, MIC-62164, |
| | cluster | MIC-87084 |
| 1557 | Tailocin gene | MIC-83010, MIC-46385, MIC-62164, |
| | cluster | MIC-87084 |
| 1558 | Tailocin gene | MIC-68773 |
| | cluster | |
| 1559 | Tailocin gene | MIC-83740 |
| | cluster | |
| 1560 | Tailocin gene | MIC-83740 |
| | cluster | |
| 1561 | Tailocin gene | MIC-83740 |
| | cluster | |
| 1562 | Tailocin gene | MIC-83740 |
| | cluster | |
| 1563 | Tailocin gene | MIC-83740 |
| | cluster | |
| 1564 | Tailocin gene | MIC-83740 |
| | cluster | |
| 1565 | Tailocin gene | MIC-83740 |
| | cluster | |
| 1566 | Tailocin gene | MIC-83740 |
| | cluster | |
| 1567 | Tailocin gene | MIC-83740 |
| | cluster | |
| 1568 | Tailocin gene | MIC-83740 |
| | cluster | |
| 1569 | Tailocin gene | MIC-83740 |
| | cluster | |
| 1570 | Tailocin gene | MIC-80455 |
| | cluster | |
| 1571 | Tailocin gene | MIC-80455 |
| | cluster | |
| 1572 | Tailocin gene | MIC-80455 |
| | cluster | |
| 1573 | Tailocin gene | MIC-80455 |
| | cluster | |
| 1574 | Tailocin gene | MIC-80455 |
| | cluster | |
| 1575 | Tailocin gene | MIC-80455 |
| | cluster | |
| 1576 | Tailocin gene | MIC-80455 |
| | cluster | |
| 1577 | Tailocin gene | MIC-80455 |
| | cluster | |
| 1578 | Tailocin gene | MIC-80455 |
| | cluster | |
| 1579 | Tailocin gene | MIC-80455, MIC-85267 |
| | cluster | |
| 1580 | Tailocin gene | MIC-87588, MIC-86605, MIC-54642 |
| | cluster | |
| 1581 | Tailocin gene | MIC-87588, MIC-86605, MIC-54642 |
| | cluster | |
| 1582 | Tailocin gene | MIC-87588, MIC-86605, MIC-54642 |
| | cluster | |
| 1583 | Tailocin gene | MIC-87588, MIC-86605, MIC-54642 |
| | cluster | |
| 1584 | Tailocin gene | MIC-87588, MIC-86605, MIC-54642 |
| | cluster | |
| 1585 | Tailocin gene | MIC-87588, MIC-86605, MIC-54642 |
| | cluster | |
| 1586 | Tailocin gene | MIC-87588, MIC-86605, MIC-54642 |
| | cluster | |
| 1587 | Tailocin gene | MIC-87588, MIC-86605, MIC-54642 |
| | cluster | |
| 1588 | Tailocin gene | MIC-87588, MIC-86605, MIC-54642 |
| | cluster | |
| 1589 | Tailocin gene | MIC-87588, MIC-86605, MIC-54642 |
| | cluster | |
| 1590 | Tailocin gene | MIC-87588, MIC-86605, MIC-54642 |
| | cluster | |
| 1591 | Tailocin gene | MIC-87588, MIC-86605, MIC-54642 |
| | cluster | |
| 1592 | Tailocin gene | MIC-87588, MIC-86605, MIC-54642 |
| | cluster | |
| 1593 | Tailocin gene | MIC-87588, MIC-86605, MIC-54642, |
| | cluster | MIC-84492 |
| 1594 | Tailocin gene | MIC-87588, MIC-86605, MIC-54642, |
| | cluster | MIC-85267 |
| 1595 | Tailocin gene | MIC-86605 |
| | cluster | |
| 1596 | Tailocin gene | MIC-86605 |
| | cluster | |
| 1597 | Tailocin gene | MIC-86605, MIC-94135, MIC-36497, |
| | cluster | MIC-82689 |
| 1598 | Tailocin gene | MIC-94504 |
| | cluster | |
| 1599 | Tailocin gene | MIC-94504 |
| | cluster | |
| 1600 | Tailocin gene | MIC-24837, MIC-55579 |
| | cluster | |
| 1601 | Tailocin gene | MIC-24837, MIC-55579 |
| | cluster | |
| 1602 | Tailocin gene | MIC-24837, MIC-55579 |
| | cluster | |
| 1603 | Tailocin gene | MIC-24837, MIC-55579 |
| | cluster | |
| 1604 | Tailocin gene | MIC-24837, MIC-55579 |
| | cluster | |
| 1605 | Tailocin gene | MIC-24837, MIC-55579 |
| | cluster | |
| 1606 | Tailocin gene | MIC-24837, MIC-55579 |
| | cluster | |
| 1607 | Tailocin gene | MIC-24837, MIC-55579 |
| | cluster | |
| 1608 | Tailocin gene | MIC-24837, MIC-55579 |
| | cluster | |
| 1609 | Tailocin gene | MIC-24837, MIC-55579 |
| | cluster | |
| 1610 | Tailocin gene | MIC-24837, MIC-55579 |
| | cluster | |
| 1611 | Tailocin gene | MIC-24837, MIC-55579 |
| | cluster | |
| 1612 | Tailocin gene | MIC-24837, MIC-55579 |
| | cluster | |
| 1613 | Tailocin gene | MIC-24837, MIC-55579 |
| | cluster | |
| 1614 | Tailocin gene | MIC-24837, MIC-55579 |
| | cluster | |
| 1615 | Tailocin gene | MIC-24837, MIC-55579 |
| | cluster | |
| 1616 | Tailocin gene | MIC-24837, MIC-55579 |
| | cluster | |
| 1617 | Tailocin gene | MIC-24837, MIC-55579 |
| | cluster | |
| 1618 | Tailocin gene | MIC-24837, MIC-55579 |
| | cluster | |
| 1619 | Tailocin gene | MIC-24837, MIC-55579 |
| | cluster | |
| 1620 | Tailocin gene | MIC-24837, MIC-55579 |
| | cluster | |
| 1621 | Tailocin gene | MIC-24837, MIC-55579 |
| | cluster | |
| 1622 | Tailocin gene | MIC-24837, MIC-55579 |
| | cluster | |
| 1623 | Tailocin gene | MIC-24837, MIC-55579 |
| | cluster | |
| 1624 | Tailocin gene | MIC-24837, MIC-55579 |
| | cluster | |
| 1625 | Tailocin gene | MIC-24837, MIC-55579 |
| | cluster | |
| 1626 | Tailocin gene | MIC-24837, MIC-55579 |
| | cluster | |
| 1627 | Tailocin gene | MIC-24837, MIC-55579 |
| | cluster | |
| 1628 | Tailocin gene | MIC-24837, MIC-55579 |
| | cluster | |
| 1629 | Tailocin gene | MIC-24837, MIC-55579 |
| | cluster | |
| 1630 | Tailocin gene | MIC-24837, MIC-55579 |
| | cluster | |
| 1631 | Tailocin gene | MIC-24837, MIC-55579, MIC-84492, |
| | cluster | MIC-50391, MIC-69701, MIC-82689 |
| 1632 | Tailocin gene | MIC-24837, MIC-55579, MIC-85267 |
| | cluster | |
| 1633 | Tailocin gene | MIC-24837, MIC-55579, MIC-85267 |
| | cluster | |
| 1634 | Tailocin gene | MIC-24837, MIC-55579, MIC-85267 |
| | cluster | |
| 1635 | Tailocin gene | MIC-24837, MIC-55579, MIC-85267, |
| | cluster | MIC-29285 |
| 1636 | Tailocin gene | MIC-55579 |
| | cluster | |
| 1637 | Tailocin gene | MIC-87198, MIC-94458, MIC-30352, |
| | cluster | MIC-82867 |
| 1638 | Tailocin gene | MIC-19845 |
| | cluster | |
| 1639 | Tailocin gene | MIC-19845 |
| | cluster | |
| 1640 | Tailocin gene | MIC-19845 |
| | cluster | |
| 1641 | Tailocin gene | MIC-19845 |
| | cluster | |
| 1642 | Tailocin gene | MIC-19845 |
| | cluster | |
| 1643 | Tailocin gene | MIC-19845 |
| | cluster | |
| 1644 | Tailocin gene | MIC-19845 |
| | cluster | |
| 1645 | Tailocin gene | MIC-19845 |
| | cluster | |
| 1646 | Tailocin gene | MIC-19845 |
| | cluster | |
| 1647 | Tailocin gene | MIC-19845 |
| | cluster | |
| 1648 | Tailocin gene | MIC-19845 |
| | cluster | |
| 1649 | Tailocin gene | MIC-84492 |
| | cluster | |
| 1650 | Tailocin gene | MIC-84492 |
| | cluster | |
| 1651 | Tailocin gene | MIC-84492 |
| | cluster | |
| 1652 | Tailocin gene | MIC-84492 |
| | cluster | |
| 1653 | Tailocin gene | MIC-84492 |
| | cluster | |
| 1654 | Tailocin gene | MIC-84492 |
| | cluster | |
| 1655 | Tailocin gene | MIC-84492 |
| | cluster | |
| 1656 | Tailocin gene | MIC-84492 |
| | cluster | |
| 1657 | Tailocin gene | MIC-84492 |
| | cluster | |
| 1658 | Tailocin gene | MIC-84492 |
| | cluster | |
| 1659 | Tailocin gene | MIC-84492 |
| | cluster | |
| 1660 | Tailocin gene | MIC-84492 |
| | cluster | |
| 1661 | Tailocin gene | MIC-84492 |
| | cluster | |
| 1662 | Tailocin gene | MIC-84492 |
| | cluster | |
| 1663 | Tailocin gene | MIC-84492 |
| | cluster | |
| 1664 | Tailocin gene | MIC-84492 |
| | cluster | |
| 1665 | Tailocin gene | MIC-84492 |
| | cluster | |
| 1666 | Tailocin gene | MIC-84492 |
| | cluster | |
| 1667 | Tailocin gene | MIC-84492 |
| | cluster | |
| 1668 | Tailocin gene | MIC-84492 |
| | cluster | |
| 1669 | Tailocin gene | MIC-84492, MIC-29285 |
| | cluster | |
| 1670 | Tailocin gene | MIC-50391, MIC-69701 |
| | cluster | |
| 1671 | Tailocin gene | MIC-50391, MIC-69701 |
| | cluster | |
| 1672 | Tailocin gene | MIC-50391, MIC-69701 |
| | cluster | |
| 1673 | Tailocin gene | MIC-50391, MIC-69701 |
| | cluster | |
| 1674 | Tailocin gene | MIC-50391, MIC-69701 |
| | cluster | |
| 1675 | Tailocin gene | MIC-50391, MIC-69701 |
| | cluster | |
| 1676 | Tailocin gene | MIC-50391, MIC-69701 |
| | cluster | |
| 1677 | Tailocin gene | MIC-50391, MIC-69701 |
| | cluster | |
| 1678 | Tailocin gene | MIC-50391, MIC-69701 |
| | cluster | |
| 1679 | Tailocin gene | MIC-50391, MIC-69701, MIC-29285 |
| | cluster | |
| 1680 | Tailocin gene | MIC-85267 |
| | cluster | |
| 1681 | Tailocin gene | MIC-85267 |
| | cluster | |
| 1682 | Tailocin gene | MIC-85267 |
| | cluster | |
| 1683 | Tailocin gene | MIC-85267 |
| | cluster | |
| 1684 | Tailocin gene | MIC-85267 |
| | cluster | |
| 1685 | Tailocin gene | MIC-85267 |
| | cluster | |
| 1686 | Tailocin gene | MIC-85267 |
| | cluster | |
| 1687 | Tailocin gene | MIC-85267 |
| | cluster | |
| 1688 | Tailocin gene | MIC-85267 |
| | cluster | |
| 1689 | Tailocin gene | MIC-85267 |
| | cluster | |
| 1690 | Tailocin gene | MIC-85267 |
| | cluster | |
| 1691 | Tailocin gene | MIC-85267 |
| | cluster | |
| 1692 | Tailocin gene | MIC-85267 |
| | cluster | |
| 1693 | Tailocin gene | MIC-85267 |
| | cluster | |
| 1694 | Tailocin gene | MIC-85267 |
| | cluster | |
| 1695 | Tailocin gene | MIC-85267 |
| | cluster | |
| 1696 | Tailocin gene | MIC-85267 |
| | cluster | |
| 1697 | Tailocin gene | MIC-85267 |
| | cluster | |
| 1698 | Tailocin gene | MIC-85267 |
| | cluster | |
| 1699 | Tailocin gene | MIC-85267 |
| | cluster | |
| 1700 | Tailocin gene | MIC-85267 |
| | cluster | |
| 1701 | Tailocin gene | MIC-85267 |
| | cluster | |
| 1702 | Tailocin gene | MIC-85267 |
| | cluster | |
| 1703 | Tailocin gene | MIC-85267 |
| | cluster | |
| 1704 | Tailocin gene | MIC-85267 |
| | cluster | |
| 1705 | Tailocin gene | MIC-85267 |
| | cluster | |
| 1706 | Tailocin gene | MIC-85267 |
| | cluster | |
| 1707 | Tailocin gene | MIC-85267 |
| | cluster | |
| 1708 | Tailocin gene | MIC-85267 |
| | cluster | |
| 1709 | Tailocin gene | MIC-85267 |
| | cluster | |
| 1710 | Tailocin gene | MIC-85267 |
| | cluster | |
| 1711 | Tailocin gene | MIC-85267 |
| | cluster | |
| 1712 | Tailocin gene | MIC-85267 |
| | cluster | |
| 1713 | Tailocin gene | MIC-85267 |
| | cluster | |
| 1714 | Tailocin gene | MIC-85267 |
| | cluster | |
| 1715 | Tailocin gene | MIC-85267 |
| | cluster | |
| 1716 | Tailocin gene | MIC-85267 |
| | cluster | |
| 1717 | Tailocin gene | MIC-85267, MIC-94458, MIC-30352, |
| | cluster | MIC-82867 |
| 1718 | Tailocin gene | MIC-75437, MIC-20446 |
| | cluster | |
| 1719 | Tailocin gene | MIC-75437, MIC-20446 |
| | cluster | |
| 1720 | Tailocin gene | MIC-75437, MIC-20446 |
| | cluster | |
| 1721 | Tailocin gene | MIC-75437, MIC-20446 |
| | cluster | |
| 1722 | Tailocin gene | MIC-75437, MIC-20446 |
| | cluster | |
| 1723 | Tailocin gene | MIC-75437, MIC-20446 |
| | cluster | |
| 1724 | Tailocin gene | MIC-75437, MIC-20446 |
| | cluster | |
| 1725 | Tailocin gene | MIC-75437, MIC-20446 |
| | cluster | |
| 1726 | Tailocin gene | MIC-75437, MIC-20446 |
| | cluster | |
| 1727 | Tailocin gene | MIC-75437, MIC-20446 |
| | cluster | |
| 1728 | Tailocin gene | MIC-75437, MIC-20446 |
| | cluster | |
| 1729 | Tailocin gene | MIC-75437, MIC-20446 |
| | cluster | |
| 1730 | Tailocin gene | MIC-75437, MIC-20446 |
| | cluster | |
| 1731 | Tailocin gene | MIC-38993 |
| | cluster | |
| 1732 | Tailocin gene | MIC-38993 |
| | cluster | |
| 1733 | Tailocin gene | MIC-38993 |
| | cluster | |
| 1734 | Tailocin gene | MIC-38993 |
| | cluster | |
| 1735 | Tailocin gene | MIC-38993 |
| | cluster | |
| 1736 | Tailocin gene | MIC-38993 |
| | cluster | |
| 1737 | Tailocin gene | MIC-38993 |
| | cluster | |
| 1738 | Tailocin gene | MIC-38993 |
| | cluster | |
| 1739 | Tailocin gene | MIC-38993 |
| | cluster | |
| 1740 | Tailocin gene | MIC-38993 |
| | cluster | |
| 1741 | Tailocin gene | MIC-38993 |
| | cluster | |
| 1742 | Tailocin gene | MIC-38993 |
| | cluster | |
| 1743 | Tailocin gene | MIC-38993 |
| | cluster | |
| 1744 | Tailocin gene | MIC-38993, MIC-94458, MIC-30352, |
| | cluster | MIC-82867, MIC-29285 |
| 1745 | Tailocin gene | MIC-38993, MIC-29285 |
| | cluster | |
| 1746 | Tailocin gene | MIC-94135 |
| | cluster | |
| 1747 | Tailocin gene | MIC-94135 |
| | cluster | |
| 1748 | Tailocin gene | MIC-94135 |
| | cluster | |
| 1749 | Tailocin gene | MIC-94135 |
| | cluster | |
| 1750 | Tailocin gene | MIC-94135 |
| | cluster | |
| 1751 | Tailocin gene | MIC-94135 |
| | cluster | |
| 1752 | Tailocin gene | MIC-94135 |
| | cluster | |
| 1753 | Tailocin gene | MIC-94135 |
| | cluster | |
| 1754 | Tailocin gene | MIC-94135 |
| | cluster | |
| 1755 | Tailocin gene | MIC-94135 |
| | cluster | |
| 1756 | Tailocin gene | MIC-94135 |
| | cluster | |
| 1757 | Tailocin gene | MIC-94458, MIC-30352, MIC-82867 |
| | cluster | |
| 1758 | Tailocin gene | MIC-94458, MIC-30352, MIC-82867 |
| | cluster | |
| 1759 | Tailocin gene | MIC-94458, MIC-30352, MIC-82867 |
| | cluster | |
| 1760 | Tailocin gene | MIC-94458, MIC-30352, MIC-82867 |
| | cluster | |
| 1761 | Tailocin gene | MIC-94458, MIC-30352, MIC-82867 |
| | cluster | |
| 1762 | Tailocin gene | MIC-94458, MIC-30352, MIC-82867 |
| | cluster | |
| 1763 | Tailocin gene | MIC-94458, MIC-30352, MIC-82867 |
| | cluster | |
| 1764 | Tailocin gene | MIC-94458, MIC-30352, MIC-82867 |
| | cluster | |
| 1765 | Tailocin gene | MIC-94458, MIC-30352, MIC-82867 |
| | cluster | |
| 1766 | Tailocin gene | MIC-94458, MIC-30352, MIC-82867 |
| | cluster | |
| 1767 | Tailocin gene | MIC-94458, MIC-30352, MIC-82867 |
| | cluster | |
| 1768 | Tailocin gene | MIC-94458, MIC-30352, MIC-82867 |
| | cluster | |
| 1769 | Tailocin gene | MIC-94458, MIC-30352, MIC-82867 |
| | cluster | |
| 1770 | Tailocin gene | MIC-94458, MIC-30352, MIC-82867 |
| | cluster | |
| 1771 | Tailocin gene | MIC-94458, MIC-30352, MIC-82867 |
| | cluster | |
| 1772 | Tailocin gene | MIC-94458, MIC-30352, MIC-82867 |
| | cluster | |
| 1773 | Tailocin gene | MIC-94458, MIC-30352, MIC-82867 |
| | cluster | |
| 1774 | Tailocin gene | MIC-94458, MIC-30352, MIC-82867 |
| | cluster | |
| 1775 | Tailocin gene | MIC-94458, MIC-30352, MIC-82867 |
| | cluster | |
| 1776 | Tailocin gene | MIC-94458, MIC-30352, MIC-82867 |
| | cluster | |
| 1777 | Tailocin gene | MIC-94458, MIC-30352, MIC-82867 |
| | cluster | |
| 1778 | Tailocin gene | MIC-94458, MIC-30352, MIC-82867 |
| | cluster | |
| 1779 | Tailocin gene | MIC-94458, MIC-30352, MIC-82867 |
| | cluster | |
| 1780 | Tailocin gene | MIC-94458, MIC-30352, MIC-82867 |
| | cluster | |
| 1781 | Tailocin gene | MIC-94458, MIC-30352, MIC-82867 |
| | cluster | |
| 1782 | Tailocin gene | MIC-94458, MIC-30352, MIC-82867 |
| | cluster | |
| 1783 | Tailocin gene | MIC-94458, MIC-30352, MIC-82867 |
| | cluster | |
| 1784 | Tailocin gene | MIC-94458, MIC-30352, MIC-82867 |
| | cluster | |
| 1785 | Tailocin gene | MIC-94458, MIC-30352, MIC-82867 |
| | cluster | |
| 1786 | Tailocin gene | MIC-94458, MIC-30352, MIC-82867 |
| | cluster | |
| 1787 | Tailocin gene | MIC-94458, MIC-30352, MIC-82867 |
| | cluster | |
| 1788 | Tailocin gene | MIC-94458, MIC-30352, MIC-82867 |
| | cluster | |
| 1789 | Tailocin gene | MIC-94458, MIC-30352, MIC-82867 |
| | cluster | |
| 1790 | Tailocin gene | MIC-94458, MIC-30352, MIC-82867 |
| | cluster | |
| 1791 | Tailocin gene | MIC-94458, MIC-30352, MIC-82867 |
| | cluster | |
| 1792 | Tailocin gene | MIC-94458, MIC-30352, MIC-82867 |
| | cluster | |
| 1793 | Tailocin gene | MIC-94458, MIC-30352, MIC-82867 |
| | cluster | |
| 1794 | Tailocin gene | MIC-94458, MIC-30352, MIC-82867 |
| | cluster | |
| 1795 | Tailocin gene | MIC-46385, MIC-62164 |
| | cluster | |
| 1796 | Tailocin gene | MIC-46385, MIC-62164 |
| | cluster | |
| 1797 | Tailocin gene | MIC-46385, MIC-62164 |
| | cluster | |
| 1798 | Tailocin gene | MIC-46385, MIC-62164 |
| | cluster | |
| 1799 | Tailocin gene | MIC-46385, MIC-62164 |
| | cluster | |
| 1800 | Tailocin gene | MIC-46385, MIC-62164 |
| | cluster | |
| 1801 | Tailocin gene | MIC-46385, MIC-62164 |
| | cluster | |
| 1802 | Tailocin gene | MIC-46385, MIC-62164 |
| | cluster | |
| 1803 | Tailocin gene | MIC-46385, MIC-62164, MIC-36497 |
| | cluster | |
| 1804 | Tailocin gene | MIC-36254 |
| | cluster | |
| 1805 | Tailocin gene | MIC-36254 |
| | cluster | |
| 1806 | Tailocin gene | MIC-36254 |
| | cluster | |
| 1807 | Tailocin gene | MIC-36254 |
| | cluster | |
| 1808 | Tailocin gene | MIC-36254 |
| | cluster | |
| 1809 | Tailocin gene | MIC-14970, MIC-11290 |
| | cluster | |
| 1810 | Tailocin gene | MIC-14970, MIC-11290 |
| | cluster | |
| 1811 | Tailocin gene | MIC-14970, MIC-11290 |
| | cluster | |
| 1812 | Tailocin gene | MIC-14970, MIC-11290 |
| | cluster | |
| 1813 | Tailocin gene | MIC-14970, MIC-11290 |
| | cluster | |
| 1814 | Tailocin gene | MIC-14970, MIC-11290 |
| | cluster | |
| 1815 | Tailocin gene | MIC-14970, MIC-11290 |
| | cluster | |
| 1816 | Tailocin gene | MIC-14970, MIC-11290 |
| | cluster | |
| 1817 | Tailocin gene | MIC-14970, MIC-11290 |
| | cluster | |
| 1818 | Tailocin gene | MIC-14970, MIC-11290 |
| | cluster | |
| 1819 | Tailocin gene | MIC-14970, MIC-11290 |
| | cluster | |
| 1820 | Tailocin gene | MIC-14970, MIC-11290 |
| | cluster | |
| 1821 | Tailocin gene | MIC-14970, MIC-11290 |
| | cluster | |
| 1822 | Tailocin gene | MIC-14970, MIC-11290 |
| | cluster | |
| 1823 | Tailocin gene | MIC-14970, MIC-11290 |
| | cluster | |
| 1824 | Tailocin gene | MIC-14970, MIC-11290 |
| | cluster | |
| 1825 | Tailocin gene | MIC-14970, MIC-11290 |
| | cluster | |
| 1826 | Tailocin gene | MIC-14970, MIC-11290 |
| | cluster | |
| 1827 | Tailocin gene | MIC-14970, MIC-11290 |
| | cluster | |
| 1828 | Tailocin gene | MIC-14970, MIC-11290 |
| | cluster | |
| 1829 | Tailocin gene | MIC-87084 |
| | cluster | |
| 1830 | Tailocin gene | MIC-87084 |
| | cluster | |
| 1831 | Tailocin gene | MIC-87084 |
| | cluster | |
| 1832 | Tailocin gene | MIC-87084 |
| | cluster | |
| 1833 | Tailocin gene | MIC-87084 |
| | cluster | |
| 1834 | Tailocin gene | MIC-87084 |
| | cluster | |
| 1835 | Tailocin gene | MIC-87084 |
| | cluster | |
| 1836 | Tailocin gene | MIC-36497 |
| | cluster | |
| 1837 | Tailocin gene | MIC-36497 |
| | cluster | |
| 1838 | Tailocin gene | MIC-36497 |
| | cluster | |
| 1839 | Tailocin gene | MIC-36497 |
| | cluster | |
| 1840 | Tailocin gene | MIC-36497 |
| | cluster | |
| 1841 | Tailocin gene | MIC-36497 |
| | cluster | |
| 1842 | Tailocin gene | MIC-36497 |
| | cluster | |
| 1843 | Tailocin gene | MIC-36497 |
| | cluster | |
| 1844 | Tailocin gene | MIC-36497 |
| | cluster | |
| 1845 | Tailocin gene | MIC-36497 |
| | cluster | |
| 1846 | Tailocin gene | MIC-36497 |
| | cluster | |
| 1847 | Tailocin gene | MIC-36497 |
| | cluster | |
| 1848 | Tailocin gene | MIC-36497 |
| | cluster | |
| 1849 | Tailocin gene | MIC-36497 |
| | cluster | |
| 1850 | Tailocin gene | MIC-36497 |
| | cluster | |
| 1851 | Tailocin gene | MIC-36497 |
| | cluster | |
| 1852 | Tailocin gene | MIC-36497 |
| | cluster | |
| 1853 | Tailocin gene | MIC-36497 |
| | cluster | |
| 1854 | Tailocin gene | MIC-29285 |
| | cluster | |
| 1855 | Tailocin gene | MIC-29285 |
| | cluster | |
| 1856 | Tailocin gene | MIC-29285 |
| | cluster | |
| 1857 | Tailocin gene | MIC-29285 |
| | cluster | |
| 1858 | Tailocin gene | MIC-29285 |
| | cluster | |
| 1859 | Tailocin gene | MIC-29285 |
| | cluster | |
| 1860 | Tailocin gene | MIC-29285 |
| | cluster | |
| 1861 | Tailocin gene | MIC-29285 |
| | cluster | |
| 1862 | Tailocin gene | MIC-29285 |
| | cluster | |
| 1863 | Tailocin gene | MIC-29285 |
| | cluster | |
| 1864 | Tailocin gene | MIC-29285 |
| | cluster | |
| 1865 | Tailocin gene | MIC-29285 |
| | cluster | |
| 1866 | Tailocin gene | MIC-29285 |
| | cluster | |
| 1867 | Tailocin gene | MIC-29285 |
| | cluster | |
| 1868 | Tailocin gene | MIC-29285 |
| | cluster | |
| 1869 | Tailocin gene | MIC-29285 |
| | cluster | |
| 1870 | Tailocin gene | MIC-29285 |
| | cluster | |
| 1871 | Tailocin gene | MIC-29285 |
| | cluster | |
| 1872 | Tailocin gene | MIC-29285 |
| | cluster | |
| 1873 | Tailocin gene | MIC-29285 |
| | cluster | |
| 1874 | Tailocin gene | MIC-29285 |
| | cluster | |
| 1875 | Tailocin gene | MIC-29285 |
| | cluster | |
| 1876 | Tailocin gene | MIC-29285 |
| | cluster | |
| 1877 | Tailocin gene | MIC-29285 |
| | cluster | |
| 1878 | Tailocin gene | MIC-29285 |
| | cluster | |
| 1879 | Tailocin gene | MIC-29285 |
| | cluster | |
| 1880 | Tailocin gene | MIC-29285 |
| | cluster | |
| 1881 | Tailocin gene | MIC-29285 |
| | cluster | |
| 1882 | Tailocin gene | MIC-29285 |
| | cluster | |
| 1883 | Tailocin gene | MIC-29285 |
| | cluster | |
| 1884 | Tailocin gene | MIC-29285 |
| | cluster | |
| 1885 | Tailocin gene | MIC-29285 |
| | cluster | |
| 1886 | Tailocin gene | MIC-29285 |
| | cluster | |
| 1887 | Tailocin gene | MIC-29285 |
| | cluster | |
| 1888 | Tailocin gene | MIC-29285 |
| | cluster | |
| 1889 | Tailocin gene | MIC-29285 |
| | cluster | |
| 1890 | Tailocin gene | MIC-29285 |
| | cluster | |
| 1891 | Tailocin gene | MIC-29285 |
| | cluster | |
| 1892 | Tailocin gene | MIC-29285 |
| | cluster | |
| 1893 | Tailocin gene | MIC-29285 |
| | cluster | |
| 1894 | Tailocin gene | MIC-29285 |
| | cluster | |
| 1895 | Tailocin gene | MIC-29285 |
| | cluster | |
| 1896 | Tailocin gene | MIC-29285 |
| | cluster | |
| 1897 | Tailocin gene | MIC-29285 |
| | cluster | |
| 1898 | Tailocin gene | MIC-29285 |
| | cluster | |
| 1899 | Tailocin gene | MIC-29285 |
| | cluster | |
| 1900 | Tailocin gene | MIC-29285 |
| | cluster | |
| 1901 | Tailocin gene | MIC-29285 |
| | cluster | |
| 1902 | Tailocin gene | MIC-29285 |
| | cluster | |
| 1903 | Tailocin gene | MIC-29285 |
| | cluster | |
| 1904 | Tailocin gene | MIC-29285 |
| | cluster | |
| 1905 | Tailocin gene | MIC-29285 |
| | cluster | |
| 1906 | Tailocin gene | MIC-29285 |
| | cluster | |
| 1907 | Tailocin gene | MIC-29285 |
| | cluster | |
| 1908 | Tailocin gene | MIC-29285 |
| | cluster | |
| 1909 | Tailocin gene | MIC-29285 |
| | cluster | |
| 1910 | Tailocin gene | MIC-29285 |
| | cluster | |
| 1911 | Tailocin gene | MIC-82689 |
| | cluster | |
| 1912 | Tailocin gene | MIC-82689 |
| | cluster | |
| 1913 | Tailocin gene | MIC-82689 |
| | cluster | |
| 1914 | Tailocin gene | MIC-82689 |
| | cluster | |
| 1915 | Tailocin gene | MIC-82689 |
| | cluster | |
| 1916 | Tailocin gene | MIC-82689 |
| | cluster | |
| 1917 | Tailocin gene | MIC-82689 |
| | cluster | |
| 1918 | Tailocin gene | MIC-82689 |
| | cluster | |
| 1919 | Tailocin gene | MIC-82689 |
| | cluster | |
| 1920 | Tailocin gene | MIC-82689 |
| | cluster | |
| 1921 | Tailocin gene | MIC-82689 |
| | cluster | |
| 1922 | Tailocin gene | MIC-82689 |
| | cluster | |
| 1923 | Type VI | MIC-80455, MIC-87588, MIC-86605, |
| | secretion | MIC-54642, MIC-94504, MIC-24837, |
| | system | MIC-55579, MIC-61954, MIC-29662, |
| | | MIC-50391, MIC-69701, MIC-90405, |
| | | MIC-38993, MIC-46385, MIC-62164, |
| | | MIC-87894, MIC-52924, MIC-14970, |
| | | MIC-11290, MIC-36497 |
| 1924 | Type VI | MIC-87588, MIC-86605, MIC-54642, |
| | secretion | MIC-24837, MIC-55579, MIC-61954, |
| | system | MIC-29662, MIC-90405, MIC-36497, |
| | | MIC-82689 |
| 1925 | Type VI | MIC-87588, MIC-86605, MIC-54642, |
| | secretion | MIC-61954, MIC-29662 |
| | system | |
| 1926 | Type VI | MIC-87588, MIC-86605, MIC-54642, |
| | secretion | MIC-61954, MIC-29662, MIC-50391, |
| | system | MIC-69701, MIC-90405, MIC-38993, |
| | | MIC-46385, MIC-62164, MIC-87894, |
| | | MIC-52924, MIC-36497 |
| 1927 | Type VI | MIC-70076, MIC-73019, MIC-73547 |
| | secretion | |
| | system | |
| 1928 | Type VI | MIC-70076, MIC-73019, MIC-73547 |
| | secretion | |
| | system | |
| 1929 | Type VI | MIC-70076, MIC-73019, MIC-73547 |
| | secretion | |
| | system | |
| 1930 | Type VI | MIC-70076, MIC-73019, MIC-73547 |
| | secretion | |
| | system | |
| 1931 | Type VI | MIC-68901, MIC-19814 |
| | secretion | |
| | system | |
| 1932 | Type VI | MIC-68773, MIC-14970, MIC-11290 |
| | secretion | |
| | system | |
| 1933 | Type VI | MIC-54778, MIC-14439, MIC-94458, |
| | secretion | MIC-30352, MIC-82867 |
| | system | |
| 1934 | Type VI | MIC-54778, MIC-14439, MIC-94458, |
| | secretion | MIC-30352, MIC-82867 |
| | system | |
| 1935 | Type VI | MIC-81265, MIC-88834 |
| | secretion | |
| | system | |
| 1936 | Type VI | MIC-68773 |
| | secretion | |
| | system | |
| 1937 | Type VI | MIC-68773 |
| | secretion | |
| | system | |
| 1938 | Type VI | MIC-68773, MIC-36497 |
| | secretion | |
| | system | |
| 1939 | Type VI | MIC-54778 |
| | secretion | |
| | system | |
| 1940 | Type VI | MIC-54778 |
| | secretion | |
| | system | |
| 1941 | Type VI | MIC-87198 |
| | secretion | |
| | system | |
| 1942 | Type VI | MIC-87198 |
| | secretion | |
| | system | |
| 1943 | Type VI | MIC-87198 |
| | secretion | |
| | system | |
| 1944 | Type VI | MIC-87198 |
| | secretion | |
| | system | |
| 1945 | Type VI | MIC-90405 |
| | secretion | |
| | system | |
| 1946 | Type VI | MIC-14439 |
| | secretion | |
| | system | |
| 1947 | Type VI | MIC-14439 |
| | secretion | |
| | system | |
| 1948 | Type VI | MIC-79613, MIC-53518 |
| | secretion | |
| | system | |
| 1949 | Type VI | MIC-83010 |
| | secretion | |
| | system | |
| 1950 | Type VI | MIC-83740 |
| | secretion | |
| | system | |
| 1951 | Type VI | MIC-80455 |
| | secretion | |
| | system | |
| 1952 | Type VI | MIC-80455 |
| | secretion | |
| | system | |
| 1953 | Type VI | MIC-80455 |
| | secretion | |
| | system | |
| 1954 | Type VI | MIC-94504 |
| | secretion | |
| | system | |
| 1955 | Type VI | MIC-94504 |
| | secretion | |
| | system | |
| 1956 | Type VI | MIC-94504 |
| | secretion | |
| | system | |
| 1957 | Type VI | MIC-24837, MIC-55579 |
| | secretion | |
| | system | |
| 1958 | Type VI | MIC-24837, MIC-55579 |
| | secretion | |
| | system | |
| 1959 | Type VI | MIC-19845 |
| | secretion | |
| | system | |
| 1960 | Type VI | MIC-84492 |
| | secretion | |
| | system | |
| 1961 | Type VI | MIC-84492 |
| | secretion | |
| | system | |
| 1962 | Type VI | MIC-84492 |
| | secretion | |
| | system | |
| 1963 | Type VI | MIC-50391, MIC-69701, MIC-52924 |
| | secretion | |
| | system | |
| 1964 | Type VI | MIC-50391, MIC-69701, MIC-52924 |
| | secretion | |
| | system | |
| 1965 | Type VI | MIC-85267 |
| | secretion | |
| | system | |
| 1966 | Type VI | MIC-75437, MIC-20446 |
| | secretion | |
| | system | |
| 1967 | Type VI | MIC-38993 |
| | secretion | |
| | system | |
| 1968 | Type VI | MIC-38993 |
| | secretion | |
| | system | |
| 1969 | Type VI | MIC-94135 |
| | secretion | |
| | system | |
| 1970 | Type VI | MIC-94458, MIC-30352, MIC-82867 |
| | secretion | |
| | system | |
| 1971 | Type VI | MIC-94458, MIC-30352, MIC-82867 |
| | secretion | |
| | system | |
| 1972 | Type VI | MIC-46385, MIC-62164, MIC-87894 |
| | secretion | |
| | system | |
| 1973 | Type VI | MIC-46385, MIC-62164, MIC-87894 |
| | secretion | |
| | system | |
| 1974 | Type VI | MIC-36254 |
| | secretion | |
| | system | |
| 1975 | Type VI | MIC-14970, MIC-11290 |
| | secretion | |
| | system | |
| 1976 | Type VI | MIC-14970, MIC-11290 |
| | secretion | |
| | system | |
| 1977 | Type VI | MIC-87084 |
| | secretion | |
| | system | |
| 1978 | Type VI | MIC-29285 |
| | secretion | |
| | system | |
| 1979 | Type VI | MIC-82689 |
| | secretion | |
| | system | |
| 1980 | Type VI | MIC-82689 |
| | secretion | |
| | system | |
| 1981 | Type VI | MIC-82689 |
| | secretion | |
| | system | |
| 1982 | Type VI | MIC-87588, MIC-86605, MIC-54642, |
| | secretion | MIC-61954, MIC-29662 |
| | system | |
| | putative | |
| | effector | |
| 1983 | Type VI | MIC-87588, MIC-86605, MIC-54642, |
| | secretion | MIC-61954, MIC-29662 |
| | system | |
| | putative | |
| | effector | |
| 1984 | Type VI | MIC-87588, MIC-86605, MIC-54642, |
| | secretion | MIC-61954, MIC-29662, MIC-90405 |
| | system | |
| | putative | |
| | effector | |
| 1985 | Type VI | MIC-70076, MIC-73019, MIC-54778, |
| | secretion | MIC-73547 |
| | system | |
| | putative | |
| | effector | |
| 1986 | Type VI | MIC-70076, MIC-73019, MIC-73547 |
| | secretion | |
| | system | |
| | putative | |
| | effector | |
| 1987 | Type VI | MIC-70076, MIC-73019, MIC-73547 |
| | secretion | |
| | system | |
| | putative | |
| | effector | |
| 1988 | Type VI | MIC-70076, MIC-73019, MIC-73547 |
| | secretion | |
| | system | |
| | putative | |
| | effector | |
| 1989 | Type VI | MIC-70076, MIC-73019, MIC-73547 |
| | secretion | |
| | system | |
| | putative | |
| | effector | |
| 1990 | Type VI | MIC-52924 |
| | secretion | |
| | system | |
| | putative | |
| | effector | |
| 1991 | Type VI | MIC-61954, MIC-29662 |
| | secretion | |
| | system | |
| | putative | |
| | effector | |
| 1992 | Type VI | MIC-68773 |
| | secretion | |
| | system | |
| | putative | |
| | effector | |
| 1993 | Type VI | MIC-68773 |
| | secretion | |
| | system | |
| | putative | |
| | effector | |
| 1994 | Type VI | MIC-68773 |
| | secretion | |
| | system | |
| | putative | |
| | effector | |
| 1995 | Type VI | MIC-68773 |
| | secretion | |
| | system | |
| | putative | |
| | effector | |
| 1996 | Type VI | MIC-54778 |
| | secretion | |
| | system | |
| | putative | |
| | effector | |
| 1997 | Type VI | MIC-54778 |
| | secretion | |
| | system | |
| | putative | |
| | effector | |
| 1998 | Type VI | MIC-54778 |
| | secretion | |
| | system | |
| | putative | |
| | effector | |
| 1999 | Type VI | MIC-87198 |
| | secretion | |
| | system | |
| | putative | |
| | effector | |
| 2000 | Type VI | MIC-87198 |
| | secretion | |
| | system | |
| | putative | |
| | effector | |
| 2001 | Type VI | MIC-87198 |
| | secretion | |
| | system | |
| | putative | |
| | effector | |
| 2002 | Type VI | MIC-87198 |
| | secretion | |
| | system | |
| | putative | |
| | effector | |
| 2003 | Type VI | MIC-90405 |
| | secretion | |
| | system | |
| | putative | |
| | effector | |
| 2004 | Type VI | MIC-90405 |
| | secretion | |
| | system | |
| | putative | |
| | effector | |
| 2005 | Type VI | MIC-14439 |
| | secretion | |
| | system | |
| | putative | |
| | effector | |
| 2006 | Type VI | MIC-14439 |
| | secretion | |
| | system | |
| | putative | |
| | effector | |
| 2007 | Type VI | MIC-80455 |
| | secretion | |
| | system | |
| | putative | |
| | effector | |
| 2008 | Type VI | MIC-80455 |
| | secretion | |
| | system | |
| | putative | |
| | effector | |
| 2009 | Type VI | MIC-80455 |
| | secretion | |
| | system | |
| | putative | |
| | effector | |
| 2010 | Type VI | MIC-80455 |
| | secretion | |
| | system | |
| | putative | |
| | effector | |
| 2011 | Type VI | MIC-87588, MIC-86605, MIC-54642 |
| | secretion | |
| | system | |
| | putative | |
| | effector | |
| 2012 | Type VI | MIC-94504 |
| | secretion | |
| | system | |
| | putative | |
| | effector | |
| 2013 | Type VI | MIC-94504 |
| | secretion | |
| | system | |
| | putative | |
| | effector | |
| 2014 | Type VI | MIC-94504 |
| | secretion | |
| | system | |
| | putative | |
| | effector | |
| 2015 | Type VI | MIC-94504, MIC-90405 |
| | secretion | |
| | system | |
| | putative | |
| | effector | |
| 2016 | Type VI | MIC-24837, MIC-55579 |
| | secretion | |
| | system | |
| | putative | |
| | effector | |
| 2017 | Type VI | MIC-24837, MIC-55579 |
| | secretion | |
| | system | |
| | putative | |
| | effector | |
| 2018 | Type VI | MIC-24837, MIC-55579 |
| | secretion | |
| | system | |
| | putative | |
| | effector | |
| 2019 | Type VI | MIC-24837, MIC-55579 |
| | secretion | |
| | system | |
| | putative | |
| | effector | |
| 2020 | Type VI | MIC-84492 |
| | secretion | |
| | system | |
| | putative | |
| | effector | |
| 2021 | Type VI | MIC-50391, MIC-69701 |
| | secretion | |
| | system | |
| | putative | |
| | effector | |
| 2022 | Type VI | MIC-50391, MIC-69701, MIC-52924 |
| | secretion | |
| | system | |
| | putative | |
| | effector | |
| 2023 | Type VI | MIC-50391, MIC-69701, MIC-52924 |
| | secretion | |
| | system | |
| | putative | |
| | effector | |
| 2024 | Type VI | MIC-50391, MIC-69701, MIC-52924 |
| | secretion | |
| | system | |
| | putative | |
| | effector | |
| 2025 | Type VI | MIC-75437, MIC-20446 |
| | secretion | |
| | system | |
| | putative | |
| | effector | |
| 2026 | Type VI | MIC-14439, MIC-94458, MIC-30352, |
| | secretion | MIC-82867 |
| | system | |
| | putative | |
| | effector | |
| 2027 | Type VI | MIC-14439, MIC-94458, MIC-30352, |
| | secretion | MIC-82867 |
| | system | |
| | putative | |
| | effector | |
| 2028 | Type VI | MIC-38993 |
| | secretion | |
| | system | |
| | putative | |
| | effector | |
| 2029 | Type VI | MIC-38993 |
| | secretion | |
| | system | |
| | putative | |
| | effector | |
| 2030 | Type VI | MIC-38993 |
| | secretion | |
| | system | |
| | putative | |
| | effector | |
| 2031 | Type VI | MIC-38993 |
| | secretion | |
| | system | |
| | putative | |
| | effector | |
| 2032 | Type VI | MIC-94458, MIC-30352, MIC-82867 |
| | secretion | |
| | system | |
| | putative | |
| | effector | |
| 2033 | Type VI | MIC-94458, MIC-30352, MIC-82867 |
| | secretion | |
| | system | |
| | putative | |
| | effector | |
| 2034 | Type VI | MIC-94458, MIC-30352, MIC-82867 |
| | secretion | |
| | system | |
| | putative | |
| | effector | |
| 2035 | Type VI | MIC-46385, MIC-62164 |
| | secretion | |
| | system | |
| | putative | |
| | effector | |
| 2036 | Type VI | MIC-46385, MIC-62164, MIC-87894 |
| | secretion | |
| | system | |
| | putative | |
| | effector | |
| 2037 | Type VI | MIC-46385, MIC-62164, MIC-87894 |
| | secretion | |
| | system | |
| | putative | |
| | effector | |
| 2038 | Type VI | MIC-46385, MIC-62164, MIC-87894 |
| | secretion | |
| | system | |
| | putative | |
| | effector | |
| 2039 | Type VI | MIC-46385, MIC-62164, MIC-87894 |
| | secretion | |
| | system | |
| | putative | |
| | effector | |
| 2040 | Type VI | MIC-14970, MIC-11290 |
| | secretion | |
| | system | |
| | putative | |
| | effector | |
| 2041 | Type VI | MIC-14970, MIC-11290 |
| | secretion | |
| | system | |
| | putative | |
| | effector | |
| 2042 | Type VI | MIC-14970, MIC-11290 |
| | secretion | |
| | system | |
| | putative | |
| | effector | |
| 2043 | Type VI | MIC-14970, MIC-11290 |
| | secretion | |
| | system | |
| | putative | |
| | effector | |
| 2044 | Type VI | MIC-36497 |
| | secretion | |
| | system | |
| | putative | |
| | effector | |
| 2045 | Type VI | MIC-36497 |
| | secretion | |
| | system | |
| | putative | |
| | effector | |
| 2046 | Type VI | MIC-36497 |
| | secretion | |
| | system | |
| | putative | |
| | effector | |
| 2047 | Type VI | MIC-36497 |
| | secretion | |
| | system | |
| | putative | |
| | effector | |
| 2048 | Type VI | MIC-82689 |
| | secretion | |
| | system | |
| | putative | |
| | effector | |
| 2049 | Type VI | MIC-82689 |
| | secretion | |
| | system | |
| | putative | |
| | effector | |
| 2050 | Type VI | MIC-82689 |
| | secretion | |
| | system | |
| | putative | |
| | effector | |
| 2051 | Type VI | MIC-82689 |
| | secretion | |
| | system | |
| | putative | |
| | effector | |

### Example 3. Assessment of Improved Plant Characteristics: Vigor Assay

### Assay of Soy Seedling Vigor

Seed preparation: The lot quality of soybean seeds is first assessed by testing germination of 100 seeds. Seeds are placed, 8 seeds per petri dish, on filter paper in petri dishes, 12 ml of water is added to each plate and plates are incubated for 3 days at 24° C. The process should be repeated with a fresh seed lot if fewer than 95% of the seeds have germinated. One thousand soybean seeds are then surface sterilized by co-incubation with chlorine gas in a 20×30 cm container placed in a chemical fume hood for 16 hours. Percent germination of 50 seeds, per sterilization batch, is tested as above and confirmed to be greater than 95%.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per soy seed (˜10\hat{ }3 CFUs/seed is obtained). Control treatments are prepared by adding equivalent volumes of sterile water to seeds.

Assay of seedling vigor: Two rolled pieces of germination paper are placed in a sterile glass gar with 50 ml sterile water, then removed when completely saturated. Then the papers are separated, and inoculated seeds are placed at approximately 1 cm intervals along the length of one sheet of moistened germination paper, at least 2.5 cm from the top of the paper and 3.8 cm from the edge of the paper. The second sheet of is placed on top of the soy seeds and the layered papers and seeds are loosely rolled into a tube. Each tube is secured with a rubber band around the middle and placed in a single sterile glass jar and covered loosely with a lid. For each treatment, three jars with 15 seeds per jar are prepared. The position of jars within the growth chamber is randomized. Jars are incubated at 60% relative humidity, and 22° C. day, 18° C. night with 12 hours light and 12 hours dark for 4 days and then the lids are removed, and the jars incubated for an additional 7 days. Then the germinated soy seedlings are weighed and photographed, and root length and root surface area are measured.

Dirt, excess water, seed coats and other debris is removed from seedlings to allow accurate scanning of the roots. Individual seedlings are laid out on clear plastic trays and trays are arranged on an Epson Expression 11000XL scanner (Epson America, Inc., Long Beach CA). Roots are manually arranged to reduce the amount of overlap. For root measurements, shoots are removed if the shape of the shoot causes it to overlap the roots.

The WinRHIZO software version Arabidopsis Pro2016a (Regents Instruments, Quebec Canada) is used with the following acquisition settings: greyscale 4000 dpi image, speed priority, overlapping (1 object), Root Morphology: Precision (standard), Crossing Detection (normal). The scanning area is set to the maximum scanner area. When the scan is completed, the root area is selected, and root length and root surface area are measured.

Statistical analysis is performed using R (R Core Team, 2016. R: A language and environment for statistical computing. R Foundation for Statistical Computing, Vienna, Austria. R-project.org/) or a similar statistical software program.

### Assay of Rice Seedling Vigor

Seed preparation: The lot of rice seeds is first evaluated for germination by transfer of 100 seeds and with 8 ml of water to a filter paper lined petri dish. Seeds are incubated for 3 days at 24° C. The process should be repeated with a fresh seed lot if fewer than 95% of the seeds have germinated. Rice seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% polyethylene glycol (PEG) is prepared by adding 75 g of PEG to 1000 ml of water, then stirring on a warm hot plate until the PEG is fully dissolved. The solution is then autoclaved.

Preparation of endophyte treatments: Spore solutions are made by rinsing and scraping spores from agar slants which have been growing for about 1 month. Rinsing was done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per rice seed (˜10\hat{ }3 CFUs/seed was obtained). Seeds and spores are combined in a 50 ml falcon tube and gently shaken for 5-10 seconds until thoroughly coated. Control treatments are prepared by adding equivalent volumes of sterile water to seeds.

Assay of seedling vigor: Petri dishes are prepared by adding four sheets of sterile heavy weight seed germination paper, then adding either 50 ml of sterile water or, optionally, 50 ml of PEG solution as prepared above, to each plate then allowing the liquid to thoroughly soak into all sheets. The sheets are positioned and then creased so that the back of the plate and one side wall are covered, two sheets are then removed and placed on a sterile surface. Along the edge of the plate across from the covered side wall 15 inoculated rice seeds are placed evenly at least one inch from the top of the plate and half an inch from the sides. Seeds are placed smooth side up and with the pointed end of the seed pointing toward the side wall of the plate covered by germination paper. The seeds are then covered by the two reserved sheets, and the moist paper layers smoothed together to remove air bubbles and secure the seeds, and then the lid is replaced. For each treatment, at least three plates with 15 seeds per plate are prepared. The plates are then randomly distributed into stacks of 8-12 plates and a plate without seeds is placed on the top. The stacks are incubated at 60% relative humidity, and 22° C. day, 18° C. night with 12 hours light and 12 hours dark for 24 hours, then each plate is turned to a semi-vertical position with the side wall covered by paper at the bottom. The plates are incubated for an additional 5 days, then rice seeds are scored manually for germination, root and shoot length.

Statistical analysis is performed using R or a similar statistical software program.

### Assay of Corn Seedling Vigor

Seed preparation: The lot quality of corn seeds is first evaluated for germination by transfer of 100 seeds with 3.5 ml of water to a filter paper lined petri dish. Seeds are incubated for 3 days at 24° C. The process should be repeated with a fresh seed lot if fewer than 95% of the seeds have germinated. One thousand corn seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% PEG 6000 (Calbiochem, San Diego, CA) is prepared by adding 75 g of PEG to 1000 ml of water, then stirred on a warm hot plate until the PEG is fully dissolved. The solution is then autoclaved.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per corn seed (˜10\hat{ }3 CFUs/seed is obtained). Control treatments are prepared by adding equivalent volumes of sterile water to seeds.

Assay of seedling vigor: Either 25 ml of sterile water or, optionally, 25 ml of PEG solution as prepared above, is added to each Cyg™ germination pouch (Mega International, Newport, MN) and place into pouch rack (Mega International, Newport, MN). Sterile forceps are used to place corn seeds prepared as above into every other perforation in the germination pouch. Seeds are fitted snugly into each perforation to ensure they do not shift when moving the pouches. Before and in between treatments forceps are sterilized using ethanol and flame and workspace wiped down with 70% ethanol. For each treatment, three pouches with 15 seeds per pouch are prepared. The germination racks with germination pouches are placed into plastic tubs and covered with perforated plastic wrap to prevent drying. Tubs are incubated at 60% relative humidity, and 22° C. day, 18° C. night with 12 hours light and 12 hours dark for 6 days to allow for germination and root length growth. Placement of pouches within racks and racks/tubs within the growth chamber is randomized to minimize positional effect. At the end of 6 days the corn seeds are scored manually for germination, root and shoot length.

Statistical analysis is performed using R or a similar statistical software program.

### Assay of Wheat Seedling Vigor

Seed preparation: The lot of wheat seeds is first evaluated for germination by transfer of 100 seeds and with 8 ml of water to a filter paper lined petri dish. Seeds are incubated for 3 days at 24° C. The process should be repeated with a fresh seed lot if fewer than 95% of the seeds have germinated. Wheat seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% polyethylene glycol (PEG) is prepared by adding 75 g of PEG to 1000 ml of water, then stirring on a warm hot plate until the PEG is fully dissolved. The solution is then autoclaved.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per wheat seed (˜10\hat{ }3 CFUs/seed was obtained). Seeds and spores are combined in a 50 ml falcon tube and gently shaken for 5-10 seconds until thoroughly coated. Control treatments are prepared by adding equivalent volumes of sterile water to seeds.

Assay of seedling vigor: Petri dishes are prepared by adding four sheets of sterile heavy weight seed germination paper, then adding either 50 ml of sterile water or, optionally, 50 ml of PEG solution as prepared above, to each plate then allowing the liquid to thoroughly soak into all sheets. The sheets are positioned and then creased so that the back of the plate and one side wall are covered, two sheets are then removed and placed on a sterile surface. Along the edge of the plate across from the covered side wall 15 inoculated wheat seeds are placed evenly at least one inch from the top of the plate and half an inch from the sides. Seeds are placed smooth side up and with the pointed end of the seed pointing toward the side wall of the plate covered by germination paper. The seeds are then covered by the two reserved sheets, and the moist paper layers smoothed together to remove air bubbles and secure the seeds, and then the lid is replaced. For each treatment, at least three plates with 15 seeds per plate are prepared. The plates are then randomly distributed into stacks of 8-12 plates and a plate without seeds is placed on the top. The stacks are incubated at 60% relative humidity, and 22° C. day, 18° C. night with 12 hours light and 12 hours dark for 24 hours, then each plate is turned to a semi-vertical position with the side wall covered by paper at the bottom. The plates are incubated for an additional 5 days, then wheat seeds are scored manually for scored manually for germination, root and shoot length, root and shoot surface area, seedling mass, root and shoot and seedling length.

Statistical analysis is performed using R or a similar statistical software program.

### Example 3A. Assessment of Improved Traits of Agronomic Importance: Reduced Pathogen Growth

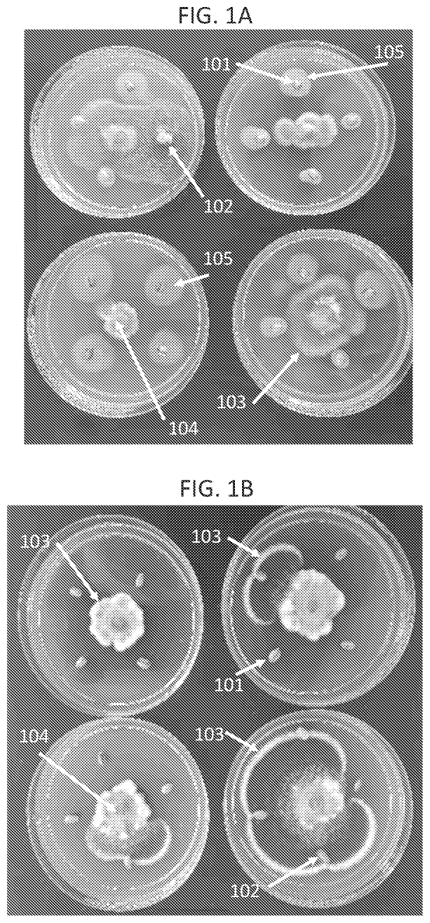

Petri dishes were prepared with sterile growth medium. Each plate was inoculated with a plug of pathogen placed in the center of the plate (for example see FIG. 1A, 104), and surrounded by four autoclaved wheat seeds treated with MIC-70076 or chemical fungicide or untreated controls (lacking formulation and the one or more heterologously disposed endophyte) as described in Example 4; MIC-70076 was applied to seeds at a rate of 0.65 mL/kg seed. Plates were incubated and pathogen growth monitored.

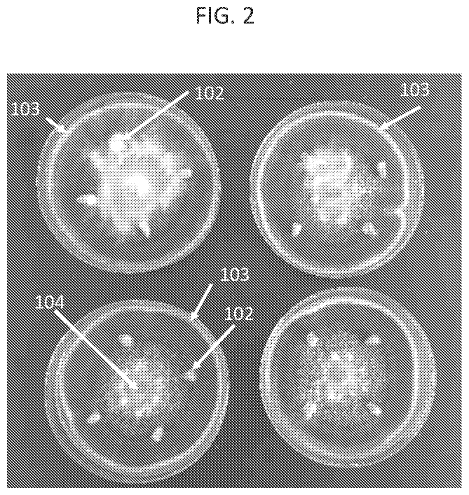

FIG. 1A, FIG. 1B, and FIG. 2 show exemplary images of plates inoculated with Dreschlera tritici-repentis. FIG. 1A shows an exemplary image of a plate inoculated with Dreschlera tritici-repentis and four autoclaved wheat seeds treated with MIC-70076 (0.65 ml/kg). FIG. 1A shows that a visible biofilm has formed around the treated seed (105), a halo of inhibition has formed around each seed, and the area of Dreschlera tritici-repentis growth on the plate is limited. FIG. 1B shows an exemplary image of a plate inoculated with Dreschlera tritici-repentis and four autoclaved wheat seeds treated with chemical fungicides Thiram and Carbendazim. FIG. 1B also shows the area of Dreschlera tritici-repentis growth on the plate is limited. FIG. 2 shows an exemplary image of a plate inoculated with Dreschlera tritici-repentis and four wheat seeds not treated with any endophyte or chemical fungicide.

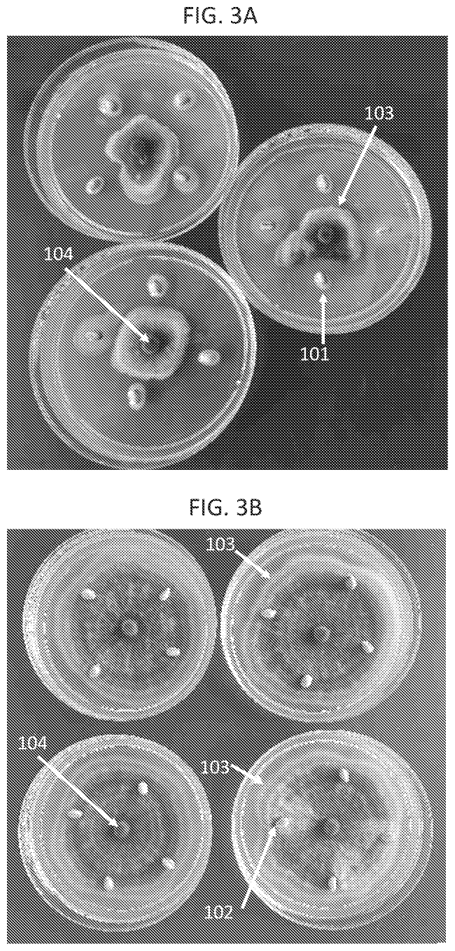

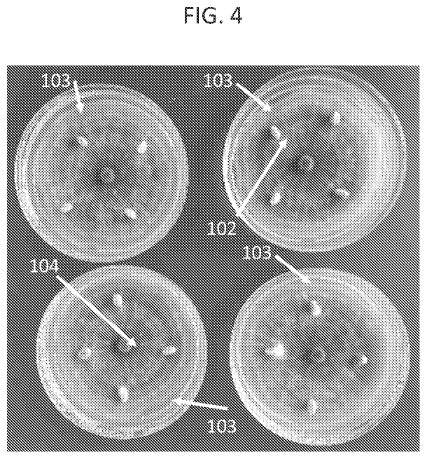

FIG. 3A shows exemplary images of plates inoculated with Bipolaris sorokiniana. FIG. 3A shows an exemplary image of a plate inoculated with Bipolaris sorokiniana and autoclaved four wheat seeds treated with MIC-70076 (0.65 ml/kg). FIG. 3A shows that a halo of inhibition has formed around each seed, and the area of Bipolaris sorokiniana growth on the plate is limited. FIG. 3B shows an exemplary image of a plate inoculated with Bipolaris sorokiniana and four wheat seeds treated with chemical fungicides Thiram and Carbendazim. FIG. 3B also shows the area of Bipolaris sorokiniana is not significantly reduced relative to the untreated control in FIG. 4, the Thiram and Carbendazim treatment did not reduce the area of Bipolaris sorokiniana growth on the plate. FIG. 4 shows an exemplary image of a plate inoculated with Bipolaris sorokiniana and four autoclaved wheat seeds not treated with any endophyte or chemical fungicide.

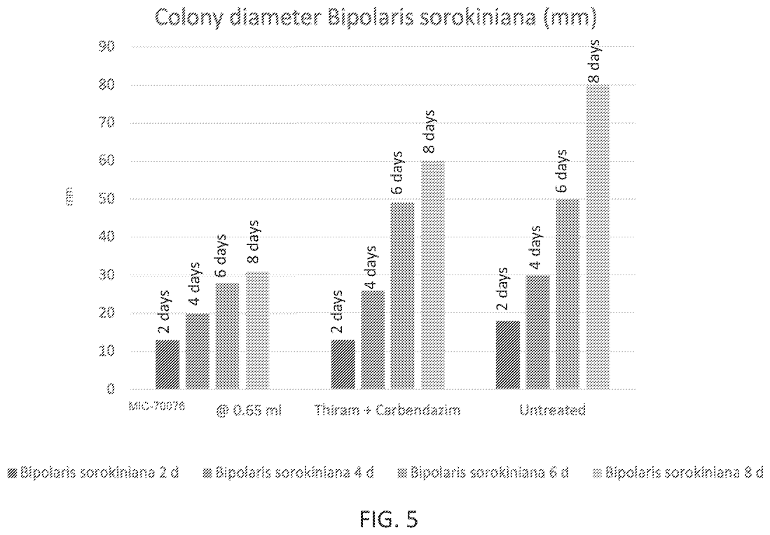

FIG. 5 shows the average colony diameter (in mm) of plates inoculated with Bipolaris sorokiniana by days of incubation and seed treatment. Three seed treatments are shown, from left to right: results from plates containing seeds treated with 0.65 ml/kg MIC-70076, seeds treated with chemical fungicides Thiram and Carbendazim, and untreated seeds. For each of the three treatments, bars represent the diameter in millimeters of the Bipolaris sorokiniana colony, from left to right, after 2, 4, 6, and 8 days of incubation.

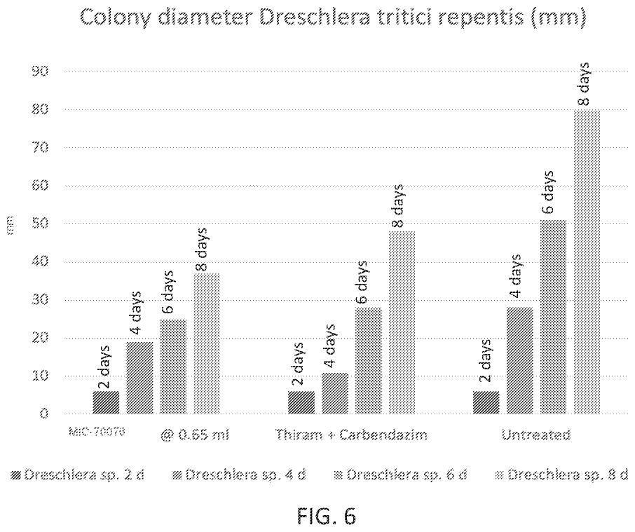

FIG. 6 shows the average colony diameter (in mm) of plates inoculated with Dreschlera tritici-repentis by days of incubation and seed treatment. Three seed treatments are shown, from left to right: results from plates containing seeds treated with 0.65 ml/kg MIC-70076, seeds treated with chemical fungicides Thiram and Carbendazim, and untreated seeds. For each of the three treatments, bars represent the diameter in millimeters of the Dreschlera tritici-repentis colony, from left to right, after 2, 4, 6, and 8 days of incubation.

### Example 4. Method of Preparation of Endophytes and Heterologous Disposition of Endophytes on Seeds for Greenhouse Trials

Seeds are heterologously disposed to each endophyte according to the following seed treatment protocol.

### Preparation of Seeds

Sieves are used to standardize the size of seeds used for greenhouse trials. The average weight of seeds is calculated by weighing 3 samples of 100 size selected seeds each and calculating the average weight of a seed. This value is used to calculate the target dose of endophyte per seed. The target dose is generally between 10\hat{ }2-10\hat{ }8 CFU per seed, in some cases at least 10\hat{ }3 CFU per seed, or at least 10\hat{ }5 CFU per seed.

### Preparation of Bacterial and Fungal Endophytes

An agar plug of each bacterial strain is transferred using a transfer tube to 4 ml of potato dextrose broth (PDB) in a 24 well plate and incubated at room temperature at 675 rpm on a shaker for 3 days. After growth of bacteria in broth, 200 μl is transferred into a spectrophotometer reading plate and bacteria OD is read at 600 nm absorbance. The total volume of inoculum needed to treat seeds with the desired dose is calculated. The target dose is generally between 10\hat{ }2-10\hat{ }8 CFU per seed, in some cases at least 10\hat{ }3 CFU per seed, or at least 10\hat{ }5 CFU per seed. The inoculum is diluted with sterile 1x PBS so that the total volume of inoculum per seed is about 2.5 microliters/seed for corn, about 1.5 microliters/seed for wheat and soy, and about 1.5 microliters/seed for cotton. Control treatments were prepared using equivalent volumes of sterile 1×PBS. The bacteria inoculum solution is applied to the prepared seeds and mixed well.

The thawed contents of a cryovial are plated on 100% MEA with 3% agar plates. The plates are sealed with Parafilm® and incubated in a growth chamber set at 60% relative humidity and 22 degrees C. with diurnal light settings (12:12 dark to light) for approximately 14 days.

Spore suspension buffer is prepared by mixing 1 ml 10% silwet with 250 ml 1×PBS and filter sterilizing. For each plate of fungi, 4-5 ml of the prepared sterile PBS is added and an L-shaped spreader used to vigorously scrape the spores, tilting the plate to allow the suspension to sink to the bottom of the plate. If additional plates of the fungal endophyte were prepared, an additional 2 ml of the prepared sterile PBS and the suspension from the prior plate of the SYM are added and the scraping procedure followed as above. The suspension is then pipetted onto a piece of sterile Miracloth over a sterile collection container. Spores per ml are counted under a microscope using a hemocytometer. The total spore suspension needed to treat the seeds with the desired dose is calculated. The target dose is generally between 10\hat{ }2-10\hat{ }8 CFU per seed, in some cases at least 10\hat{ }3 CFU per seed, or at least 10\hat{ }5 CFU per seed. The spore suspension is diluted with sterile 1x PBS so that the total volume of inoculum per seed is about 2.5 μl/seed for corn, about 1.5 μl/seed for wheat and soy, and about 1.5 μl/seed for cotton. Control treatments were prepared using equivalent volumes of sterile 1×PBS. The fungal inoculum solution is applied to the prepared seeds and mixed well.

### Example 5. Greenhouse Assessment of Improved Plant Characteristics Under Water Deficit

This example describes an exemplary method by which improved plant health of endophyte treated plants may be shown in a growth environment comprising a water deficit.

Greenhouse assay setup: This greenhouse assay is conducted in individual plastic pots, filled with moistened potting soil. This greenhouse assay is conducted using seeds (optionally, chemically treated) coated with one or more endophytes described herein and formulation control (lacking the one or more heterologously disposed endophytes) and untreated controls (lacking formulation and the one or more heterologously disposed endophyte) as described in Example 4. Seeds are placed onto each pot and lightly covered with potting mix. Replicated pots of each treatment are set up and placed on a greenhouse bench using a random block design. For example, 18 replicates are planted for each treatment and control. Plants are monitored daily for emergence and watered as necessary to maintain a moist but not saturated soil surface (for example, plants are watered with 125 ml Hoagland's solution (8 mM N) (Hoagland, D. R. and D. I. Arnon. 1950. The water culture method for growing plants without soil. California Agri. Exp. Sta. Cir. No. 347. University of California Berkeley Press, CA., pp: 347) per pot on every Monday, Wednesday and Friday).

The following growth and vigor metrics are measured for each treatment: percentage emergence at Day 4, 5, 7 (for soybean, winter wheat and cotton) or Day 3, 4, 5 (for corn), leaf count (the number of fully expanded leaves on the main stem) at Days 10, 17 and 24.

At Day 14 after planting, the potting mix in each pot is fully saturated (for example, 150 ml Hoagland's solution is added to each pot and the soil given time to fully absorb the solution, before an additional 150 ml Hoagland's solution). On subsequent days plants are observed and assigned a wilt score. Wilt scores range from 1-9 and are more fully described in the following table.

TABLE A
Description of phenotypes for each wilt scores

| Wilt | |
|---|---|
| score | Description of wilt phenotype |
| 9 | Normal no wilting - turgid green healthy |
| 8 | Leaves start losing turgor but are not soft yet no |
| | folding or rolling or change of leaf surface some small |
| | area of leaves becomes pale between the minor veins |
| 7 | Leaves further lose turgor become soft and pale at least |
| | one leaf starts slightly rolling |
| 6 | Leaves are further soft and pale all leaves are rolling |
| | except the center growing leaf |
| 5 | All leaves are very soft and pale with rolling - one leaf |
| | may be completely closed |
| 4 | Whole plant looks very bad - center leaves are very pale |
| | and rolling badly - all leaves may be completely |
| | closed - leaf sheath starts losing turgor |
| 3 | Leaf base is still fresh - leaf sheath loses turgor 2 |
| | lower leaves start drying |
| 2 | Center leaf starts drying - leaf base is not fresh |
| | anymore - all leaves are dried |
| 1 | For any plant that is worse than score 2 - the wilting |
| | score will be 1 |

Watering is withheld until 80% of plants have a wilt score of at least 4. Pots are then fully saturated and a normal watering schedule resumed. Additional vigor and growth metrics may be measured during recovery including shoot height, area of chlorosis, turgor pressure of leaves, number of live leaves, etc. After a recovery period, for example 1 week, plants are gently removed from pots, washed with tap water to remove dirt, and photographed. Optionally, plant tissue is collected for nutrient composition analysis. Plants are put into a paper bag and dried in an oven. Optionally, the plant is separated into shoot and root tissue prior to drying. The dry weight of each individual plant, or shoot or root thereof, is recorded.

### Example 6. Greenhouse Assessment of Improved Plant Characteristics Under Nitrogen Deficit

This example describes an exemplary method by which improved plant health of endophyte treated plants may be shown in a growth environment comprising a nitrogen deficit.

Greenhouse assay setup: This greenhouse assay is conducted in individual plastic pots, filled with moistened potting soil. This greenhouse assay is conducted using seeds (optionally, chemically treated) coated with one or more endophytes described herein and formulation control (lacking the one or more heterologously disposed endophytes) and untreated controls (lacking formulation and the one or more heterologously disposed endophyte) as described in Example 4. Seeds are placed onto each pot and lightly covered with potting mix. Replicated pots of each treatment are set up and placed on a greenhouse bench using a random block design. For example, 18 replicates are planted for each treatment and control. Nitrogen deficit is introduced by reducing the Nitrogen in the Hoagland's solution (3 mM N), which is used to water the plants. Plants are monitored daily for emergence and watered as necessary to maintain a moist but not saturated soil surface (for example, plants are watered with 125 ml Hoagland's solution (3 mM N) per pot on every Monday, Wednesday and Friday).

The following growth and vigor metrics are collected for each treatment: percentage emergence at Day 4, 5, 7 (for soybean, winter wheat and cotton) or Day 3, 4, 5 (for corn), leaf count (the number of fully expanded leaves on the main stem) at Days 10, 17 and 24.

Additional vigor and growth metrics may be collected including shoot height, leaf area, number of chlorotic leaves, chlorophyll content, number of live leaves, etc. At harvest plants are gently removed from pots, washed with tap water to remove dirt, and photographed. Plant tissue is collected for nutrient composition analysis. Plants are put into a paper bag and dried in an oven. Optionally, the plant is separated into shoot and root tissue prior to drying. The dry weight of each individual plant, or shoot or root thereof, is recorded.

### Example 7. Greenhouse Assessment of Improved Plant Characteristics Under Phosphorus Deficit

This example describes an exemplary method by which improved plant health of endophyte treated plants may be shown in a growth environment comprising a phosphorus deficit.

This greenhouse assay is conducted in individual plastic pots, filled with moistened potting soil. This greenhouse assay is conducted using seeds (optionally, chemically treated) coated with one or more endophytes described herein and formulation control (lacking the one or more heterologously disposed endophytes) and untreated controls (lacking formulation and the one or more heterologously disposed endophyte) as described in Example 4. Seeds are placed onto each pot and lightly covered with potting mix. Replicated pots of each treatment are set up and placed on a greenhouse bench using a random block design. For example, 16 replicates are planted for each treatment and control. Phosphorus deficit is introduced by removing Phosphorus from the Hoagland's solution (0 mM P), which is used to water the plants. Plants are monitored daily for emergence and watered as necessary to maintain a moist but not saturated soil surface (for example, plants are watered with 125 ml Hoagland's solution (0 mM P) per pot on every Monday, Wednesday and Friday).

The following growth and vigor metrics are collected for each treatment: percentage emergence at Day 4, 5, 7 (for soybean, winter wheat and cotton) or Day 3, 4, 5 (for corn), leaf count (the number of fully expanded leaves on the main stem) at Days 10, 17 and 24.

Additional vigor and growth metrics may be collected including shoot height, leaf area, coloration of leaves, number of live leaves, etc. At harvest plants are gently removed from pots, washed with tap water to remove dirt, and photographed. Plant tissue is collected for nutrient composition analysis. Plants are put into a paper bag and dried in an oven. Optionally, the plant is separated into shoot and root tissue prior to drying. The dry weight of each individual plant, or shoot or root thereof, is recorded.

### Example 8. Greenhouse Assessment of Improved Plant Health Under Biotic Stress

This example describes an exemplary method by which improved plant health of endophyte treated plants was shown in a growth environment comprising the crop pathogen Rhizoctonia solani or Pythium ultimum, causal agents of seedling damping off disease. This assay may utilize dicots or monocots, though results for soybean, cotton and wheat are described here.

Preparation of pathogen inoculum A stock of Rhizoctonia solani anastomosis group 4 and a stock of Pythium ultimum var. ultimum were each grown on a standard potato dextrose agar plate. Plugs of fresh mycelium were then transferred into standard potato dextrose broth. After sufficient growth was achieved, the cultures were poured though cheesecloth to capture the fungal biomass, which was subsequently rinsed with water. After removing excess rinse water, a roughly equivalent volume of water was added to the fungal biomass before blending to create slurries. The resulting slurries were further diluted to the required concentration necessary to observe desired level of symptoms.

Greenhouse assay setup The greenhouse assay was conducted in a commercial potting mix. A divot was placed in the center of a pot containing wetted soil using a standardized dibble. An appropriate volume of the relevant slurry was added to the center of each divot.

This greenhouse assay was conducted using seeds coated with one or more endophytes described herein and formulation control (lacking the one or more heterologously disposed endophytes) and untreated controls (lacking the one or more heterologously disposed endophyte) as described in Example 4. Concentration of endophyte treatment applied to seeds ranged from approximately 10\hat{ }2-10\hat{ }8 CFU/seed (1E6 cells/mL to 1E8 cells/mL). Seeds were placed onto each divot after addition of the relevant inoculum. The seeds were then covered with uninoculated soil and again watered. High soil moisture levels were maintained throughout the course of the experiment. Replicates were included in a randomized design to obtain sufficient statistical power for analysis. Plants were grown in a controlled environment until 7 days after planting, approximately 4 days post emergence of control plants. At this point fresh shoot weight was measured on a per plant basis.

TABLE 3
Greenhouse testing of endophytes in Rhizoctonia solani treated winter wheat, showing
% uplift (improvement) in shoot fresh weight in Rhizoctonia conditions relative to
untreated formulation controls. Each row represents an experimental trial, where each
trial contains 14 plants per treatment. Measurements were taken 7 days after planting.

| | | Average | Standard | | |
|---|---|---|---|---|---|
| | Average | Endophyte | Deviation | % Improvement | |
| | Formulation | Treatment | Endophyte | Treatment | Experiment Stress |
| MIC-ID | Control (g) | (g) | Treatment | vs. Control | Level |
| MIC-70076 | 0.058 | 0.089 | 0.016 | 52.22 | Moderate Stress |
| MIC-70076 | 0.052 | 0.043 | 0.012 | −16.17 | Moderate Stress |
| MIC-70076 | 0.077 | 0.056 | 0.011 | −27.24 | Moderate Stress |
| MIC-70076 | 0.054 | 0.019 | 0.012 | −65.63 | Moderate Stress |
| MIC-70076 | 0.027 | 0.061 | 0.013 | 123.07 | Severe Stress |
| MIC-70076 | 0.055 | 0.083 | 0.011 | 52.29 | Severe Stress |
| MIC-70076 | 0.020 | 0.014 | 0.010 | −26.94 | Severe Stress |
| MIC-73019 | 0.044 | 0.049 | 0.013 | 11.88 | Severe Stress |
| MIC-73019 | 0.037 | 0.034 | 0.014 | −7.26 | Severe Stress |
| MIC-79613 | 0.072 | 0.085 | 0.017 | 18.12 | Moderate Stress |
| MIC-82330 | 0.072 | 0.086 | 0.011 | 19.12 | Moderate Stress |
| MIC-82330 | 0.054 | 0.029 | 0.013 | −46.04 | Moderate Stress |
| MIC-82330 | 0.027 | 0.040 | 0.011 | 46.77 | Severe Stress |
| MIC-82330 | 0.055 | 0.057 | 0.011 | 4.21 | Severe Stress |
| MIC-82330 | 0.044 | 0.040 | 0.010 | −7.84 | Severe Stress |
| MIC-83010 | 0.061 | 0.077 | 0.015 | 26.57 | Moderate Stress |
| MIC-83010 | 0.027 | 0.035 | 0.013 | 27.3 | Severe Stress |
| MIC-68773 | 0.061 | 0.061 | 0.012 | 0.12 | Moderate Stress |
| MIC-68773 | 0.040 | 0.051 | 0.017 | 27.75 | Severe Stress |
| MIC-68773 | 0.053 | 0.036 | 0.010 | −30.76 | Severe Stress |
| MIC-54778 | 0.058 | 0.087 | 0.014 | 48.64 | Moderate Stress |
| MIC-54778 | 0.037 | 0.041 | 0.015 | 13.2 | Severe Stress |
| MIC-68901 | 0.026 | 0.067 | 0.016 | 154.38 | Severe Stress |
| MIC-68901 | 0.027 | 0.036 | 0.014 | 32.25 | Severe Stress |
| MIC-19814 | 0.078 | 0.067 | 0.017 | −14.11 | Moderate Stress |
| MIC-19814 | 0.027 | 0.040 | 0.009 | 48.02 | Severe Stress |
| MIC-80455 | 0.058 | 0.065 | 0.013 | 11.29 | Moderate Stress |
| MIC-80455 | 0.052 | 0.051 | 0.010 | −2.17 | Moderate Stress |
| MIC-87588 | 0.027 | 0.050 | 0.013 | 84.01 | Severe Stress |
| MIC-87588 | 0.026 | 0.036 | 0.013 | 38.41 | Severe Stress |
| MIC-86605 | 0.037 | 0.066 | 0.015 | 80.27 | Severe Stress |
| MIC-86605 | 0.044 | 0.055 | 0.013 | 27.08 | Severe Stress |
| MIC-54642 | 0.078 | 0.062 | 0.017 | −19.96 | Moderate Stress |
| MIC-54642 | 0.040 | 0.084 | 0.019 | 107.67 | Severe Stress |
| MIC-24837 | 0.061 | 0.067 | 0.016 | 10.35 | Moderate Stress |
| MIC-24837 | 0.052 | 0.035 | 0.012 | −31.55 | Moderate Stress |
| MIC-87198 | 0.078 | 0.083 | 0.019 | 6.27 | Moderate Stress |
| MIC-87198 | 0.037 | 0.035 | 0.013 | −4.14 | Severe Stress |
| MIC-29662 | 0.078 | 0.045 | 0.014 | −42.45 | Moderate Stress |
| MIC-29662 | 0.031 | 0.074 | 0.015 | 143.5 | Severe Stress |
| MIC-81265 | 0.058 | 0.050 | 0.015 | −13.36 | Moderate Stress |
| MIC-81265 | 0.040 | 0.055 | 0.016 | 37.42 | Severe Stress |
| MIC-88834 | 0.044 | 0.081 | 0.014 | 85.17 | Severe Stress |
| MIC-88834 | 0.040 | 0.056 | 0.014 | 40.29 | Severe Stress |
| MIC-84492 | 0.052 | 0.060 | 0.013 | 16.4 | Moderate Stress |
| MIC-84492 | 0.058 | 0.031 | 0.013 | −46.54 | Moderate Stress |
| MIC-90405 | 0.078 | 0.062 | 0.015 | −19.87 | Moderate Stress |
| MIC-90405 | 0.031 | 0.025 | 0.010 | −18.42 | Severe Stress |
| MIC-14439 | 0.061 | 0.037 | 0.011 | −39.74 | Moderate Stress |
| MIC-14439 | 0.031 | 0.104 | 0.018 | 240.75 | Severe Stress |
| MIC-87894 | 0.026 | 0.049 | 0.014 | 85.83 | Severe Stress |
| MIC-87894 | 0.040 | 0.041 | 0.013 | 3 | Severe Stress |
| MIC-52924 | 0.026 | 0.057 | 0.012 | 115.53 | Severe Stress |
| MIC-52924 | 0.031 | 0.050 | 0.014 | 62.67 | Severe Stress |
| MIC-73547 | 0.061 | 0.055 | 0.016 | −8.99 | Moderate Stress |
| MIC-73547 | 0.037 | 0.061 | 0.016 | 66.97 | Severe Stress |
| MIC-14970 | 0.052 | 0.043 | 0.012 | −17.09 | Moderate Stress |
| MIC-14970 | 0.044 | 0.075 | 0.017 | 71.81 | Severe Stress |

TABLE 4
Greenhouse testing of endophytes in Rhizoctonia solani treated
soybeans, showing % uplift (improvement) in shoot fresh weight in Rhizoctonia
conditions relative to untreated formulation controls. Each row represents
an experimental trial, where each trial contains 14 plants per treatment.
Measurements were taken 7 days after planting.

| | | Average | Standard | % | |
|---|---|---|---|---|---|
| | Average | Endophyte | Deviation | Improvement | Experiment |
| | Formulation | Treatment | Endophyte | Treatment | Stress |
| MIC-ID | Control (g) | (g) | Treatment | vs. Control | Level |
| MIC-70076 | 0.726 | 0.700 | 0.112 | −3.66 | Moderate Stress |
| MIC-70076 | 0.168 | 0.361 | 0.093 | 114.26 | Severe Stress |
| MIC-70076 | 0.257 | 0.440 | 0.107 | 71.41 | Severe Stress |
| MIC-70076 | 0.444 | 0.526 | 0.143 | 18.31 | Severe Stress |
| MIC-70076 | 0.380 | 0.401 | 0.104 | 5.44 | Severe Stress |
| MIC-70076 | 0.286 | 0.286 | 0.087 | 0.04 | Severe Stress |
| MIC-70076 | 0.322 | 0.249 | 0.059 | −22.64 | Severe Stress |
| MIC-70076 | 0.219 | 0.082 | 0.055 | −62.59 | Severe Stress |
| MIC-73019 | 0.280 | 0.197 | 0.084 | −29.6 | Moderate Stress |
| MIC-73019 | 0.190 | 0.222 | 0.072 | 16.97 | Severe Stress |
| MIC-79613 | 0.316 | 0.230 | 0.086 | −27.28 | Moderate Stress |
| MIC-79613 | 0.292 | 0.183 | 0.079 | −37.21 | Severe Stress |
| MIC-82330 | 0.280 | 0.283 | 0.093 | 1.17 | Moderate Stress |
| MIC-82330 | 0.444 | 0.410 | 0.118 | −7.85 | Severe Stress |
| MIC-83010 | 0.238 | 0.386 | 0.092 | 61.77 | Severe Stress |
| MIC-68773 | 0.729 | 0.812 | 0.043 | 11.45 | Mild Stress |
| MIC-68773 | 0.700 | 0.470 | 0.133 | −32.84 | Moderate Stress |
| MIC-68773 | 0.238 | 0.333 | 0.087 | 39.71 | Severe Stress |
| MIC-54778 | 0.257 | 0.384 | 0.084 | 49.63 | Severe Stress |
| MIC-54778 | 0.190 | 0.179 | 0.078 | −5.54 | Severe Stress |
| MIC-68901 | 0.230 | 0.416 | 0.131 | 80.72 | Severe Stress |
| MIC-80455 | 0.219 | 0.311 | 0.086 | 42.13 | Severe Stress |
| MIC-80455 | 0.257 | 0.296 | 0.111 | 15.26 | Severe Stress |
| MIC-87588 | 0.230 | 0.297 | 0.095 | 29.29 | Severe Stress |
| MIC-86605 | 0.280 | 0.256 | 0.082 | −8.36 | Moderate Stress |
| MIC-86605 | 0.190 | 0.125 | 0.068 | −34.01 | Severe Stress |
| MIC-24837 | 0.219 | 0.311 | 0.085 | 41.91 | Severe Stress |
| MIC-24837 | 0.238 | 0.312 | 0.085 | 30.74 | Severe Stress |
| MIC-87198 | 0.190 | 0.223 | 0.074 | 17.66 | Severe Stress |
| MIC-29662 | 0.260 | 0.403 | 0.101 | 55.15 | Moderate Stress |
| MIC-29662 | 0.529 | 0.361 | 0.090 | −31.89 | Moderate Stress |
| MIC-81265 | 0.257 | 0.220 | 0.081 | −14.25 | Severe Stress |
| MIC-88834 | 0.280 | 0.206 | 0.077 | −26.39 | Moderate Stress |
| MIC-84492 | 0.257 | 0.323 | 0.103 | 26.05 | Severe Stress |
| MIC-84492 | 0.219 | 0.231 | 0.092 | 5.68 | Severe Stress |
| MIC-90405 | 0.260 | 0.287 | 0.078 | 10.41 | Moderate Stress |
| MIC-14439 | 0.260 | 0.195 | 0.086 | −25.05 | Moderate Stress |
| MIC-14439 | 0.238 | 0.266 | 0.104 | 11.66 | Severe Stress |
| MIC-87894 | 0.529 | 0.225 | 0.085 | −57.49 | Moderate Stress |
| MIC-87894 | 0.230 | 0.297 | 0.095 | 29.33 | Severe Stress |
| MIC-87894 | 0.218 | 0.146 | 0.063 | −33.15 | Severe Stress |
| MIC-52924 | 0.260 | 0.211 | 0.092 | −18.98 | Moderate Stress |
| MIC-52924 | 0.218 | 0.320 | 0.100 | 47 | Severe Stress |
| MIC-52924 | 0.230 | 0.174 | 0.095 | −24.12 | Severe Stress |
| MIC-73547 | 0.238 | 0.447 | 0.116 | 87.36 | Severe Stress |
| MIC-73547 | 0.190 | 0.213 | 0.079 | 12.48 | Severe Stress |
| MIC-14970 | 0.280 | 0.379 | 0.091 | 35.5 | Moderate Stress |
| MIC-14970 | 0.219 | 0.055 | 0.054 | −74.77 | Severe Stress |

TABLE 5
Greenhouse testing of endophytes in Rhizoctonia solani treated corn, showing %
uplift (improvement) in shoot fresh weight in Rhizoctonia conditions relative to
untreated formulation controls. Each row represents an experimental trial, where each
trial contains 14 plants per treatment. Measurements were taken 7 days after planting.

| | | | Standard | | |
|---|---|---|---|---|---|
| | Average | Average | Deviation | % Improvement | |
| | Formulation | Endophyte | Endophyte | Treatment vs. | Experiment Stress |
| MIC-ID | Control (g) | Treatment (g) | Treatment | Control | Level |
| MIC-70076 | 0.478 | 0.585 | 0.063 | 22.41 | Moderate Stress |
| MIC-70076 | 0.326 | 0.252 | 0.026 | −22.7 | Moderate Stress |
| MIC-70076 | 0.339 | 0.261 | 0.043 | −23.23 | Moderate Stress |
| MIC-70076 | 0.310 | 0.344 | 0.040 | 10.95 | Severe Stress |
| MIC-70076 | 0.385 | 0.366 | 0.063 | −4.99 | Severe Stress |
| MIC-73019 | 0.219 | 0.224 | 0.035 | 2.36 | Severe Stress |
| MIC-79613 | 0.368 | 0.430 | 0.053 | 16.66 | Moderate Stress |
| MIC-82330 | 0.219 | 0.255 | 0.047 | 16.6 | Severe Stress |
| MIC-83010 | 0.287 | 0.230 | 0.031 | −19.62 | Moderate Stress |
| MIC-68773 | 0.287 | 0.321 | 0.047 | 12.13 | Moderate Stress |
| MIC-68901 | 0.245 | 0.229 | 0.051 | −6.55 | Moderate Stress |
| MIC-19814 | 0.316 | 0.269 | 0.040 | −15.1 | Moderate Stress |
| MIC-87588 | 0.326 | 0.291 | 0.045 | −10.62 | Moderate Stress |
| MIC-24837 | 0.245 | 0.264 | 0.031 | 8.08 | Moderate Stress |
| MIC-87198 | 0.219 | 0.283 | 0.043 | 29.14 | Severe Stress |
| MIC-61954 | 0.316 | 0.332 | 0.031 | 5.08 | Moderate Stress |
| MIC-61954 | 0.249 | 0.248 | 0.043 | −0.35 | Severe Stress |
| MIC-29662 | 0.287 | 0.202 | 0.044 | −29.47 | Moderate Stress |
| MIC-81265 | 0.316 | 0.372 | 0.045 | 17.46 | Moderate Stress |
| MIC-88834 | 0.316 | 0.228 | 0.046 | −27.83 | Moderate Stress |
| MIC-84492 | 0.326 | 0.249 | 0.042 | −23.49 | Moderate Stress |
| MIC-90405 | 0.219 | 0.289 | 0.035 | 31.88 | Severe Stress |
| MIC-14439 | 0.326 | 0.323 | 0.049 | −0.73 | Moderate Stress |
| MIC-87894 | 0.334 | 0.343 | 0.045 | 2.83 | Moderate Stress |
| MIC-87894 | 0.245 | 0.230 | 0.031 | −5.92 | Moderate Stress |
| MIC-52924 | 0.245 | 0.284 | 0.025 | 16.33 | Moderate Stress |
| MIC-52924 | 0.334 | 0.290 | 0.047 | −13.17 | Moderate Stress |
| MIC-73547 | 0.287 | 0.279 | 0.032 | −2.8 | Moderate Stress |
| MIC-14970 | 0.219 | 0.221 | 0.042 | 0.91 | Severe Stress |

TABLE 6
Greenhouse testing of endophytes in Pythium ultimum treated winter wheat, showing
% uplift (improvement) in shoot fresh weight in Pythium conditions relative to untreated
formulation controls. Each row represents an experimental trial, where each trial contains
13-14 plants per treatment. Measurements were taken 7 days after planting.

| | | | Standard | | |
|---|---|---|---|---|---|
| | Average | Average | Deviation | % Improvement | |
| | Formulation | Endophyte | Endophyte | Treatment vs. | Experiment Stress |
| MIC-ID | Control (g) | Treatment (g) | Treatment | Control | Level |
| MIC-70076 | 0.0686 | 0.1172 | 0.0128 | 70.79 | Moderate Stress |
| MIC-70076 | 0.0687 | 0.1049 | 0.0112 | 52.69 | Moderate Stress |
| MIC-70076 | 0.0670 | 0.0800 | 0.0185 | 19.4 | Moderate Stress |
| MIC-70076 | 0.0361 | 0.0879 | 0.0158 | 143.96 | Severe Stress |
| MIC-73019 | 0.0243 | 0.0853 | 0.0175 | 251.2 | Severe Stress |
| MIC-82330 | 0.0243 | 0.0859 | 0.0174 | 253.59 | Severe Stress |
| MIC-68773 | 0.0681 | 0.0316 | 0.0153 | −53.58 | Moderate Stress |
| MIC-68773 | 0.0261 | 0.0535 | 0.0089 | 104.81 | Severe Stress |
| MIC-68901 | 0.0409 | 0.0599 | 0.0165 | 46.34 | Moderate Stress |
| MIC-68901 | 0.0309 | 0.0821 | 0.0191 | 165.48 | Severe Stress |
| MIC-19814 | 0.0551 | 0.1264 | 0.0142 | 129.39 | Moderate Stress |
| MIC-87588 | 0.0309 | 0.0656 | 0.0171 | 111.97 | Severe Stress |
| MIC-24837 | 0.0409 | 0.0858 | 0.0173 | 109.47 | Moderate Stress |
| MIC-87198 | 0.0243 | 0.0836 | 0.0152 | 244.41 | Severe Stress |
| MIC-61954 | 0.0551 | 0.1257 | 0.0117 | 128.21 | Moderate Stress |
| MIC-81265 | 0.0551 | 0.1135 | 0.0184 | 106 | Moderate Stress |
| MIC-88834 | 0.0551 | 0.0718 | 0.0170 | 30.24 | Moderate Stress |
| MIC-90405 | 0.0243 | 0.0885 | 0.0163 | 264.55 | Severe Stress |
| MIC-87894 | 0.0409 | 0.1000 | 0.0171 | 144.2 | Moderate Stress |
| MIC-87894 | 0.0309 | 0.0690 | 0.0192 | 122.9 | Severe Stress |
| MIC-52924 | 0.0409 | 0.0960 | 0.0165 | 134.63 | Moderate Stress |
| MIC-52924 | 0.0309 | 0.0980 | 0.0192 | 216.66 | Severe Stress |
| MIC-14970 | 0.0243 | 0.1031 | 0.0181 | 324.39 | Severe Stress |

TABLE 7
Greenhouse testing of endophytes in Pythium ultimum treated soybeans, showing
% uplift (improvement) in shoot fresh weight in Pythium conditions relative to untreated
formulation controls. Each row represents an experimental trial, where each trial contains
14 plants per treatment. Measurements were taken 7 days after planting.

| | | | Standard | | |
|---|---|---|---|---|---|
| | Average | Average | Deviation | % Improvement | |
| | Formulation | Endophyte | Endophyte | Treatment vs. | Experiment Stress |
| MIC-ID | Control (g) | Treatment (g) | Treatment | Control | Level |
| MIC-70076 | 0.681519286 | 0.780716786 | 0.101911355 | 14.56 | Mild Stress |
| MIC-70076 | 0.499931786 | 0.541149286 | 0.132633987 | 8.24 | Moderate Stress |
| MIC-70076 | 0.567195107 | 0.444530857 | 0.148165398 | −21.63 | Moderate Stress |
| MIC-70076 | 0.431813321 | 0.119205321 | 0.076550572 | −72.39 | Moderate Stress |
| MIC-70076 | 0.376254607 | 0.102149893 | 0.068179552 | −72.85 | Moderate Stress |
| MIC-70076 | 0.219568929 | 0.216680714 | 0.08379432 | −1.32 | Severe Stress |
| MIC-70076 | 0.300859429 | 0.07093225 | 0.045564391 | −76.42 | Severe Stress |
| MIC-70076 | 0.377259071 | 0.075242571 | 0.071618867 | −80.06 | Severe Stress |
| MIC-73019 | 0.414505393 | 0.59976475 | 0.101351274 | 44.69 | Moderate Stress |
| MIC-79613 | 0.32095275 | 0.227044821 | 0.101189971 | −29.26 | Severe Stress |
| MIC-82330 | 0.566426214 | 0.733073357 | 0.1135387 | 29.42 | Moderate Stress |
| MIC-83010 | 0.566426214 | 0.745968 | 0.116391993 | 31.7 | Moderate Stress |
| MIC-83010 | 0.3529345 | 0.461483571 | 0.124803815 | 30.76 | Severe Stress |
| MIC-68773 | 0.606372286 | 0.568282 | 0.10183432 | −6.28 | Moderate Stress |
| MIC-68773 | 0.301981643 | 0.533778964 | 0.149431383 | 76.76 | Severe Stress |
| MIC-68773 | 0.3529345 | 0.27986925 | 0.11462408 | −20.7 | Severe Stress |
| MIC-54778 | 0.414505393 | 0.72064375 | 0.115995773 | 73.86 | Moderate Stress |
| MIC-54778 | 0.431813321 | 0.291673036 | 0.11259605 | −32.45 | Moderate Stress |
| MIC-68901 | 0.566426214 | 0.567824821 | 0.123225852 | 0.25 | Moderate Stress |
| MIC-68901 | 0.160529929 | 0.41745125 | 0.153202249 | 160.05 | Severe Stress |
| MIC-19814 | 0.566426214 | 0.606710643 | 0.095529637 | 7.11 | Moderate Stress |
| MIC-19814 | 0.28483625 | 0.182616643 | 0.098639515 | −35.89 | Severe Stress |
| MIC-80455 | 0.431813321 | 0.631045964 | 0.153621085 | 46.14 | Moderate Stress |
| MIC-87588 | 0.566426214 | 0.628242607 | 0.108383173 | 10.91 | Moderate Stress |
| MIC-87588 | 0.160529929 | 0.495691071 | 0.138343299 | 208.78 | Severe Stress |
| MIC-86605 | 0.414505393 | 0.475934143 | 0.132301443 | 14.82 | Moderate Stress |
| MIC-54642 | 0.409144286 | 0.558461071 | 0.112529788 | 36.49 | Moderate Stress |
| MIC-54642 | 0.28483625 | 0.511577429 | 0.143051623 | 79.6 | Severe Stress |
| MIC-24837 | 0.3529345 | 0.472880357 | 0.15260377 | 33.99 | Severe Stress |
| MIC-87198 | 0.414505393 | 0.709004571 | 0.130700051 | 71.05 | Moderate Stress |
| MIC-87198 | 0.28483625 | 0.427147821 | 0.14004676 | 49.96 | Severe Stress |
| MIC-61954 | 0.370836036 | 0.455275393 | 0.108643318 | 22.77 | Moderate Stress |
| MIC-29662 | 0.28483625 | 0.4648315 | 0.133143236 | 63.19 | Severe Stress |
| MIC-81265 | 0.431813321 | 0.335675571 | 0.145377129 | −22.26 | Moderate Stress |
| MIC-84492 | 0.431813321 | 0.566101536 | 0.135381278 | 31.1 | Moderate Stress |
| MIC-90405 | 0.28483625 | 0.552188321 | 0.126216186 | 93.86 | Severe Stress |
| MIC-14439 | 0.3529345 | 0.435111714 | 0.124928742 | 23.28 | Severe Stress |
| MIC-87894 | 0.160529929 | 0.471540036 | 0.123128327 | 193.74 | Severe Stress |
| MIC-87894 | 0.32697125 | 0.238861179 | 0.088605788 | −26.95 | Severe Stress |
| MIC-52924 | 0.160529929 | 0.310303179 | 0.122197025 | 93.3 | Severe Stress |
| MIC-52924 | 0.32697125 | 0.290218357 | 0.124847415 | −11.24 | Severe Stress |
| MIC-73547 | 0.414505393 | 0.542681357 | 0.129912886 | 30.92 | Moderate Stress |
| MIC-73547 | 0.3529345 | 0.350298036 | 0.128556685 | −0.75 | Severe Stress |

### Example 9. Soybean Cyst Nematode Preparation

The eggs of Heterodera glycines are extracted from soybean stock culture and are used as inoculum for in vitro, growth chamber, greenhouse, and microplot experiments.

In one embodiment, the following method is used. Eggs are extracted from a 60-day-old soybean stock culture maintained in, e.g., 500 ml polystyrene pots. The soil is gently washed from the soybean roots and cysts and females are dislodged from the roots. Water with the cyst and female suspension is poured through nested 850-μm-pore and 250-μm-pore sieves to separate trash from cysts and females. Cysts and females are ground with a mortar and pestle to release the eggs. Eggs are washed with water, collected on a 25-μm-pore sieve, transferred to two 50 ml centrifuge tubes, and spun for 5 minutes at 1,750 r.p.m. The supernatant liquid is then poured off and a sugar solution added (1 lb. cane sugar, 1 liter water), thoroughly mixing sugar solution and sediment. The suspension is centrifuged at 240 g for 1 minute. The supernatant containing the nematodes is poured on to the 25-μm-pore sieve. After rinsing the sugar away with water, the nematodes are ready for use. For in vitro tests, H. glycines eggs are placed in a modified Baermann funnel (Castillo J D., Lawrence K S., Kloepper J W. Biocontrol of the reniform nematode by Bacillus firmus GB126 and Paecilomyces lilacinus 251 on cotton. Plant Disease. 2013; 97:967-976) on a Slide Warmer (Model 77) (Marshall Scientific, Brentwood, NH) and incubated at 31° C. for 5 to 7 days to obtain the J2. The J2 are collected on a 25-μm-pore sieve, transferred to 1.5 ml microcentrifuge tubes, centrifuged at 5,000 g for 1 minute, rinsed with sterile distilled water, and centrifuged at 5,000 g for 1 minute. The J2 suspensions are adjusted to 30 to 40 J2 per 10 μl of water. Eggs are enumerated at 40× magnification with an inverted TS100 Nikon microscope and standardized to 2,000 eggs per 500 ml polystyrene pot.

### Example 10. Greenhouse Assessment of Improved Plant Health Under Biotic Stress (Soybean Cyst Nematode)

This example describes an exemplary method by which improved plant health of endophyte treated plants may be shown in a growth environment comprising the crop pest soybean cyst nematode (Heterodera glycines).

Greenhouse assays are conducted using soybean seeds (optionally, chemically treated soybean seeds) coated with one or more of the endophytes described herein and formulation control (lacking the one or more heterologously disposed endophytes) and untreated controls (lacking formulation and the one or more heterologously disposed endophyte) as described in Example 4. Microbe treated soybean seeds are planted, infected with nematodes, maintained, and phenotyped in grow rooms.

In one embodiment, the following method is used. 98 cones are placed in each cone-tainer to obtain the needed number of cone-tainers. Masks are placed over cones and cones are filled with soil. The cone-tainer is placed in a deep pan and water is added until the soil in the cones is saturated. Two soybean seeds are planted 2.5 cm deep in each cone-tainer. Each cone-tainer is placed in a growth tub and watered.

One ml containing 2,000 H. glycines eggs is pipetted into each cone-tainer at planting or the desired number of days after planting. Seedlings are thinned to one per cone-tainer after emergence and watered as appropriate.

Phenotyping is performed as follows. The height of each plant is measured, e.g., by placing the ruler on the lip of a cell and measuring the plant's height to the nearest millimeter. The mass of each plant is measured, e.g., by cutting the plant at the soil surface, placing the shoot in the weighing container, allowing the weight to stabilize, and autorecording the mass via the scale's software. The number of H. glycines cysts may be counted after extraction from soybean roots as described herein. The water suspension containing 150 cm\hat{ }3 of soil is poured through nested 75-μm and 25-μm-pore sieves to extract vermiform stages (juveniles and males). Vermiform stages are collected on the 75-μm-pore sieve and centrifuged using, e.g., the sucrose centrifugation-flotation method.

### Example 11. Greenhouse Assessment of Improved Plant Health Under Biotic Stress (Soybean Aphid)

This example describes an exemplary method by which improved plant health of endophyte treated plants may be shown in a growth environment comprising the crop pest soybean aphid (Aphis glycines).

Greenhouse assays are conducted using soybean seeds (optionally, chemically treated soybean seeds) coated with one or more of the endophytes described herein and formulation control (lacking the one or more heterologously disposed endophytes) and untreated controls (lacking formulation and the one or more heterologously disposed endophyte) as described in Example 4. Microbe treated soybean seeds are planted, infected with soybean aphids (Aphis glycines), maintained in grow rooms, and phenotyped.

In one embodiment, the following method is used. 98 cones are placed in each cone-tainer to obtain the needed number of cone-tainers. Masks are placed over cones and cones are filled with potting medium or soil. The cone-tainer is placed in a deep pan and water is added until the soil in the cones is saturated. One soybean seed is planted in each cone-tainer. Each cone-tainer is placed in a growth tub and watered.

A community of soybean aphids is maintained on a stock of soybean plants. To prepare for infestation of the experimental plants, leaves are removed from infested soybean plants from the stock community. One or more leaves are examined under a stereoscope to make sure the aphids are alive and vigorous. Infested leaf cutlets are placed in square plates to keep leaves alive until the treatment plants are infested with aphids. In some embodiments, 20 infested leaf cutlets are used per each 98 cone tray used in the experiment. The infested leaf cutlets are introduced to the growth environment of the experimental plants at planting or the desired number of days after planting, in some embodiments, 9 days after planting. The experimental cone-tainers are infested following an infestation pattern to allow for aphid choice feeding in planta. The infested experimental plants are maintained in their growth environment until phenotyping.

The plants may be phenotyped at one or more times after infestation, for example 1 day, 4 days, 7 days or more after infestation. Measurement of one or more traits of agronomic importance is performed as follows. The height of each plant is measured, e.g., by placing the ruler on the lip of a cell and measuring the plant's height to the nearest millimeter or using an automated tool such as a Phenospex PlantEye 3D laser scanner (Phenospex B.V., Heerlen, The Netherlands). Other traits of agronomic importance may be measured either manually or using a tool such as the Phenospex PlantEye 3D laser scanner, for example the greenness of the plants and the leaf and/or above ground plant area. The mass of each plant may be measured for example via destructive sampling, e.g., by cutting the plant at the soil surface, placing the shoot in the weighing container, allowing the weight to stabilize, and autorecording the mass via the scale's software. The experimental plants may be maintained through their reproductive stages, and traits of agronomic importance such as number of flowers, number of pods and number of seeds per pod may be measured.

### Example 12. Greenhouse Assessment of Improved Plant Health Under Biotic Stress

This example describes an exemplary method by which improved plant health of endophyte treated plants was shown in a growth environment comprising the crop pathogen Fusarium sp., one of the causal agents of seedling damping off disease. This assay may utilize dicots or monocots, including, for example, soybean and wheat as shown here.

Preparation of Fusarium sp. Inoculum. A stock of Fusarium sp. was grown on a standard potato dextrose agar plate. Plugs of fresh mycelium were then transferred into breathable bag containing a sterile mixture of water and grain such as sorghum or millet. After sufficient growth is achieved, the culture was removed from the bags and dried. After drying the biomass was coarsely ground.

Greenhouse assay setup The greenhouse assay was conducted in a media mixture consisting of a commercial potting mix and a minimum of 50% inert inorganic material. An appropriate volume of ground pathogen was added to the soil mixture to obtain moderate to severe symptoms.

This greenhouse assay was conducted using seeds coated with one or more endophytes described herein and formulation control (lacking the one or more heterologously disposed endophytes) and untreated controls (lacking the one or more heterologously disposed endophyte) as described in Example 4. Concentration of endophyte treatment applied to seeds ranged from approximately 10\hat{ }2-10\hat{ }8 CFU/seed (1E6 cells/mL to 3E8 cells/mL). A seed was added to the surface of the infested media. The seed was then covered with media lacking pathogen and again watered. High soil moisture levels were maintained throughout the course of the experiment. Replicates were included in a randomized design to obtain sufficient statistical power for analysis. Plants were grown in a controlled environment until 8-11 days after planting, approximately 4 days post emergence of control plants. At this point shoot fresh weight was measured on a per plant basis.

TABLE 8
Greenhouse testing of endophytes in Fusarium oxysporum treated soybeans, showing
% uplift (improvement) in shoot fresh weight in Fusarium conditions relative to untreated
formulation controls. Each row represents an experimental trial, where each trial contains
14 plants per treatment. Measurements were taken 10-12 days after planting.

| | | | Standard | | |
|---|---|---|---|---|---|
| | Average | Average | Deviation | % Improvement | |
| | Formulation | Endophyte | Endophyte | Treatment vs. | Experiment Stress |
| MIC-ID | Control (g) | Treatment (g) | Treatment | Control | Level |
| MIC-70076 | 0.784 | 0.713 | 0.079 | −9.110 | Mild Stress |
| MIC-70076 | 0.535 | 0.661 | 0.114 | 23.560 | Moderate Stress |
| MIC-70076 | 0.496 | 0.600 | 0.135 | 20.930 | Moderate Stress |
| MIC-70076 | 0.389 | 0.267 | 0.103 | −31.420 | Moderate Stress |
| MIC-70076 | 0.334 | 0.476 | 0.052 | 42.300 | Severe Stress |
| MIC-70076 | 0.378 | 0.420 | 0.048 | 11.150 | Severe Stress |
| MIC-82330 | 1.034 | 0.855 | 0.120 | −17.260 | Mild Stress |
| MIC-82330 | 0.171 | 0.260 | 0.053 | 52.120 | Severe Stress |
| MIC-82330 | 0.214 | 0.263 | 0.044 | 23.170 | Severe Stress |
| MIC-68773 | 0.942 | 0.751 | 0.087 | −20.310 | Mild Stress |
| MIC-87588 | 0.834 | 0.969 | 0.073 | 16.140 | Moderate Stress |
| MIC-29662 | 1.047 | 0.934 | 0.116 | −10.750 | Mild Stress |
| MIC-84492 | 0.651 | 0.822 | 0.148 | 26.290 | Moderate Stress |
| MIC-87894 | 0.751 | 0.952 | 0.040 | 26.700 | Moderate Stress |
| MIC-52924 | 0.490 | 0.683 | 0.112 | 39.310 | Severe Stress |

TABLE 9
Greenhouse testing of endophytes in Fusarium treated winter wheat, showing %
uplift (improvement) in shoot fresh weight in Fusarium oxysporum conditions
relative to untreated formulation controls. Each row represents an experimental
trial, where each trial contains 14 plants per treatment. Measurements were
taken at 7-9 days after planting.

| | | | Standard | | |
|---|---|---|---|---|---|
| | Average | Average | Deviation | % Improvement | |
| | Formulation | Endophyte | Endophyte | Treatment vs. | Experiment Stress |
| MIC-ID | Control (g) | Treatment (g) | Treatment | Control | Level |
| MIC-70076 | 0.059 | 0.081 | 0.010 | 38.630 | Moderate Stress |
| MIC-70076 | 0.047 | 0.058 | 0.005 | 24.530 | Moderate Stress |
| MIC-70076 | 0.029 | 0.036 | 0.004 | 23.110 | Moderate Stress |
| MIC-70076 | 0.037 | 0.039 | 0.004 | 6.520 | Moderate Stress |
| MIC-70076 | 0.056 | 0.059 | 0.006 | 5.790 | Moderate Stress |
| MIC-70076 | 0.021 | 0.031 | 0.003 | 44.530 | Severe Stress |
| MIC-70076 | 0.007 | 0.007 | 0.003 | 0.370 | Severe Stress |
| MIC-82330 | 0.036 | 0.044 | 0.004 | 23.710 | Moderate Stress |
| MIC-82330 | 0.049 | 0.042 | 0.005 | −13.440 | Moderate Stress |
| MIC-68773 | 0.037 | 0.046 | 0.005 | 25.460 | Moderate Stress |
| MIC-87588 | 0.034 | 0.038 | 0.006 | 9.690 | Moderate Stress |
| MIC-29662 | 0.054 | 0.056 | 0.005 | 3.930 | Moderate Stress |
| MIC-84492 | 0.070 | 0.075 | 0.008 | 7.740 | Moderate Stress |
| MIC-87894 | 0.029 | 0.017 | 0.004 | −42.610 | Severe Stress |
| MIC-52924 | 0.028 | 0.033 | 0.007 | 16.260 | Severe Stress |

### Example 13. Field Assessment of Improved Plant Health of Soy Under Biotic Stress

This example describes an exemplary method by which improved plant health of endophyte treated plants may be shown in a growth environment comprising the fungal pathogen Fusarium virguliforme.

Field trials were conducted using chemically treated soy seeds coated with MIC-70076 and untreated controls (lacking formulation and the one or more heterologously disposed endophyte) as described in Example 4. Plots are infected with Fusarium virguliforme, the causal agent of Fusarium Sudden Death Syndrome (SDS). Replicate plots, preferably at least 4 replicate plots, are planted per endophyte or control treatment in a randomized complete block design. Each plot consists of a 7.62 m (25 ft.) by 0.76 m (2.5 ft.) row. The following early growth metrics are measured: early emergence, full emergence, plant height, fresh shoot weight, and fresh root weight.

The percent uplift relative to untreated plants was calculated using R (R Core Team, 2016. R: A language and environment for statistical computing. R Foundation for Statistical Computing, Vienna, Austria. R-project.org/).

TABLE 10
Field testing of endophytes in Fusarium treated soybeans, showing
% uplift (improvement) relative to untreated controls. Confirmed stress
data points represented confirmed Fusarium stress plots.

| | | Early | Full | Plant | Root | Shoot |
|---|---|---|---|---|---|---|
| Trial | Treatment | Emergence | Emergence | Height | Weight | Weight |
| All (42 data points) | Chemical treatment | 11.2 | 5.5 | 2.5 | −1.5 | 3.5 |
| All (42 data points) | MIC-70076 | 3.3 | 1.5 | 2 | 4.4 | 6.8 |
| Confirmed Stress | Chemical treatment | 27 | 9.2 | −2.5 | −4.1 | 4.6 |
| (18 data points) | | | | | | |
| Confirmed Stress | MIC-70076 | 15 | 6.7 | −2.2 | 5.2 | 8.8 |
| (18 data points) | | | | | | |

### Example 14. Field Assessment of Improved Plant Health of Soy Under Biotic Stress

This example describes an exemplary method by which improved plant health of endophyte treated plants may be shown in a growth environment comprising the fungal pathogen Rhizoctonia solani.

Field trials were conducted using chemically treated soy seeds coated with MIC-70076 and untreated controls (lacking formulation and the one or more heterologously disposed endophyte) as described in Example 4. Plots are infected with Rhizoctonia solani. Replicate plots, preferably at least 4 replicate plots, are planted per endophyte or control treatment in a randomized complete block design. Each plot consists of a 7.62 m (25 ft.) by 0.76 m (2.5 ft.) row. The following early growth metrics are measured: early emergence, full emergence, plant height, fresh shoot weight, and fresh root weight.

The percent uplift relative to untreated plants was calculated using R (R Core Team, 2016. R: A language and environment for statistical computing. R Foundation for Statistical Computing, Vienna, Austria. R-project.org/).

TABLE 11
Field testing of endophytes in Rhizoctonia treated soybeans,
showing % uplift/improvement relative to untreated controls.

| | | | Early | Full | Plant | Root | Shoot |
|---|---|---|---|---|---|---|---|
| Year | Trial | Treatment | Emergence | Emergence | Height | Weight | Weight |
| 1 | All (12 data points) | Chemical | 12.6 | 58.3 | 2.6 | −6.4 | 13.4 |
| | | treatment | | | | | |
| | All (12 data points) | MIC-70076 | 19.6 | 19.7 | 1 | 3.7 | 6.6 |
| 2 | All (42 data points) | Chemical | 22.2 | 15.3 | 2.3 | −3.6 | 3.7 |
| | | treatment | | | | | |
| | All (42 data points) | MIC-70076 | 14 | 1.2 | −0.2 | 1.6 | −5.4 |

### Example 15. Field Assessment of Improved Plant Health of Soy Under Biotic Stress

This example describes an exemplary method by which improved plant health of endophyte treated plants may be shown in a growth environment comprising the oomycetes pathogen Pythium.

Field trials were conducted using chemically treated soy seeds coated with MIC-70076 and untreated controls (lacking formulation and the one or more heterologously disposed endophyte) as described in Example 4. Plots are infected with Pythium. Replicate plots, preferably at least 4 replicate plots, are planted per endophyte or control treatment in a randomized complete block design. Each plot consists of a 7.62 m (25 ft.) by 0.76 m (2.5 ft.) row. The following early growth metrics are measured: early emergence, full emergence, plant height, fresh shoot weight, and fresh root weight.

The percent uplift relative to untreated plants was calculated using R (R Core Team, 2016. R: A language and environment for statistical computing. R Foundation for Statistical Computing, Vienna, Austria. R-project.org/).

TABLE 12
Field testing of endophytes in Pythium treated soybeans, showing
% uplift/improvement relative to untreated controls. Confirmed stress
data points represented confirmed Pythium stress plots

| | | | Early | Full | Plant | Root | Shoot |
|---|---|---|---|---|---|---|---|
| Year | Trial | Treatment | Emergence | Emergence | Height | Weight | Weight |
| 1 | All | Chemical | −9.4 | 14.1 | 1.6 | 17 | 7.4 |
| | (12 data points) | treatment | | | | | |
| | All | MIC-70076 | 3.8 | 11.6 | −0.3 | 23.7 | 12.3 |
| | (12 data points) | | | | | | |
| 2 | All | Chemical | 4.8 | 2.9 | 0.8 | 2.3 | 7.9 |
| | (36 data points) | treatment | | | | | |
| | All | MIC-70076 | 6.4 | 3.8 | 1.3 | 5.3 | 0.3 |
| | (36 data points) | | | | | | |
| 2 | Confirmed Stress | Chemical | 10.9 | 6.6 | −1.6 | 10.6 | 6.8 |
| | (12 data points) | treatment | | | | | |
| | Confirmed Stress | MIC-70076 | 10.3 | 4.5 | −0.7 | 14.4 | −1.4 |
| | (12 data points) | | | | | | |

### Example 16. Field Assessment of Improved Plant Health of Cotton Under Biotic Stress

This example describes an exemplary method by which improved plant health of endophyte treated plants may be shown in a growth environment comprising the crop pests root knot nematode (Meloidogyne incognita), Reniform nematode (Rotylenchulus reniformis), and, opportunistically, the fungal pathogen Fusarium virguliforme.

Field trials are conducted using chemically treated cotton seeds coated with one or more of the endophytes described herein and formulation control (lacking the one or more heterologously disposed endophytes) and untreated controls (lacking formulation and the one or more heterologously disposed endophyte) as described in Example 4. Plots for in-field assessment harbor populations of root knot nematode and Reniform nematode, respectively, at an approximately 1.0+E04 eggs per gram of fresh root weight. Opportunistically, these plots are infected with natural inoculum of Fusarium virguliforme, the causal agent of Fusarium SDS. Replicate plots, preferably at least 4 replicate plots, are planted per endophyte or control treatment in a randomized complete block design. Each plot consists of a 7.62 m (25 ft.) by 0.76 m (2.5 ft.) row. The following early growth metrics are measured: percent emergence at 14 days post planting, standing count at 28 and 45 days post planting, plant vigor at 14, 28, and 45 days post planting, plant height at 45 days post planting, fresh shoot weight, fresh root weight, disease rating at a 0-3 scale (3 denotes strong disease symptoms) using the split-root scoring system at 45 days post planting, nematode count at 45 days post planting, and yield parameters.

At the end of the field trial employing endophyte treatment and control treatment plants, plants (preferably at least 4 plants) are randomly dug out from each row, kept in a plastic bag, and brought back to lab for metric measurements. For each seedling, shoot and root are separated by cutting the seedling 3 cm from the first branch of the root. The heights of the separated shoot of each plant are measured, followed by fresh shoot weight, and fresh root weight. The main root is vertically split into two halves and discoloration of xylem is scored as described above. To extract and count nematode eggs on root, roots are placed in a container prefilled with 100 ml 10% sucrose and incubated on a shaker at room temperature overnight. The supernatant is then collected and nematode eggs are counted under a stereomicroscope.

The percentage of survival plants, fresh root weight, and nematode egg count are plotted as bar graph of mean±95% confidence interval from the mean using the ggplot2 package of R (R Core Team, 2016. R: A language and environment for statistical computing. R Foundation for Statistical Computing, Vienna, Austria. R-project.org/). Plant heights, fresh shoot weight, and disease scores are plotted as jittered dot of mean±nonparametric bootstrap (1000) of 95% confidence interval from the mean using the ggplot2 package of R.

### Example 17. Field Assessment of Improved Plant Health of Winter Wheat Under Biotic Stress

This example describes a method for detection of improved plant health of endophyte treated winter wheat in a growth environment comprising the crop pathogens Rhizoctonia spp., Pythium spp., and Fusarium spp (causal agents of damping-off disease).

Field trials are conducted using winter wheat seeds coated with an endophyte of the present invention and untreated controls (lacking formulation and the heterologously disposed endophyte). Rhizoctonia, Fusarium, and Pythium inoculant are applied per standard practice to each seed packet before planting. Five replicate plots are planted per endophyte treatment and control treatment in a randomized complete block design. Each plot consists of a 6 ft. by 20 ft. block. Irrigation is applied pre-planting and in early season to maximize disease pressure. Plots are harvested by machine, and yield is calculated by the on-board computer.

### Example 18. Field Assessment of Improved Plant Health of Corn Under Biotic Stress

This example describes a method for detection of improved plant health of endophyte treated corn in a growth environment comprising the crop pathogen Fusarium spp.

Field trials were conducted using corn seeds coated with MIC-70076, a control treated with chemical fungicide (lacking formulation and the heterologously disposed endophyte), and untreated controls (lacking formulation and the heterologously disposed endophyte). Fusarium inoculant was applied per standard practice to each seed packet before planting, targeting moderate level of disease infestation; enough to affect plant stand, but not to a level resulting in total loss. Five replicate plots were planted per endophyte treatment and control treatment in a randomized complete block design. Each plot consisted of a 25 ft. long, 2-4 row block.

Plots were harvested by machine, and yield was calculated by the on-board computer.

TABLE 13
Field testing of endophytes in Fusarium treated corn, showing
% uplift/improvement relative to untreated controls. Confirmed stress
data points represented confirmed Fusarium stress plots.

| | | | Early | Full | Plant | Root | Shoot |
|---|---|---|---|---|---|---|---|
| Year | Trial | Treatment | Emergence | Emergence | Height | Weight | Weight |
| 1 | All | Chemical | 4.6 | −0.5 | 2.5 | 4.4 | 9.9 |
| | (12 data points) | treatment | | | | | |
| | All | MIC-70076 | −1 | −1.4 | 2.1 | 1.4 | 5.3 |
| | (12 data points) | | | | | | |
| 2 | All | Chemical | 8.7 | 6.3 | 2.3 | −0.3 | −1.9 |
| | (54 data points) | treatment | | | | | |
| | All | MIC-70076 | 3.2 | 1.7 | 1 | −6.1 | −4.9 |
| | (54 data points) | | | | | | |
| | Confirmed Stress | Chemical | 12.9 | 8.8 | 6.5 | 10.7 | 18.1 |
| | (36 data points) | treatment | | | | | |
| | Confirmed Stress | MIC-70076 | 5.2 | 3.3 | 6.6 | 3.6 | 3 |
| | (36 data points) | | | | | | |

### Example 19. Field Assessment of Improved Plant Health of Corn Under Biotic Stress

This example describes a method for detection of improved plant health of endophyte treated cotton in a growth environment comprising the crop pathogen Pythium spp.

Field trials were conducted using cotton seeds coated with MIC-70076, a control treated with chemical fungicide (lacking formulation and the heterologously disposed endophyte), and untreated controls (lacking formulation and the heterologously disposed endophyte). Pythium inoculant was applied per standard practice to each seed packet before planting, targeting moderate level of disease infestation; enough to affect plant stand, but not to a level resulting in total loss. Five replicate plots were planted per endophyte treatment and control treatment in a randomized complete block design. Each plot consisted of a 25 ft. long, 2-4 row block.

Plots were harvested by machine, and yield was calculated by the on-board computer.

TABLE 14
Field testing of endophytes in Pythium treated corn, showing %
uplift (improvement) relative to untreated controls. Confirmed stress
data points represented confirmed Pythium stress plots.

| | | | Early | Full | Plant | Root | Shoot |
|---|---|---|---|---|---|---|---|
| Year | Trial | Treatment | Emergence | Emergence | Height | Weight | Weight |
| 1 | All | Chemical | 19.1 | 11.5 | 2.7 | 13.1 | 13.5 |
| | (12 data points) | treatment | | | | | |
| | All | MIC-70076 | 0.9 | 1.9 | 2.2 | 9.6 | 6.8 |
| | (12 data points) | | | | | | |
| | Confirmed Stress | Chemical | 69.9 | 24.3 | 15.7 | 23 | 32.9 |
| | (6 data points) | treatment | | | | | |
| | Confirmed Stress | MIC-70076 | 4.8 | 6.1 | 9.3 | 18.2 | 18.3 |
| | (6 data points) | | | | | | |
| 2 | All | Chemical | 14.6 | 10.3 | 6.1 | 12.7 | 15.7 |
| | (48 data points) | treatment | | | | | |
| | All | MIC-70076 | 4.6 | 2.9 | −0.5 | 6.1 | 2 |
| | (48 data points) | | | | | | |

### Example 20. Field Assessment of Improved Plant Health of Soybean Under Biotic Stress

This example describes an exemplary method for detection of improved plant health of endophyte treated soybean in a growth environment comprising the crop pathogen Rhizoctonia.

Field trials were conducted using cotton seeds coated with MIC-70076, a control treated with chemical fungicide (lacking formulation and the heterologously disposed endophyte), and untreated controls (lacking formulation and the heterologously disposed endophyte). Rhizoctonia inoculant was applied per standard practice to each seed packet before planting, targeting moderate level of disease infestation; enough to affect plant stand, but not to a level resulting in total loss. At least four replicate plots were planted per endophyte treatment and control treatment in a randomized complete block design. Each plot consisted of approximately a 25 ft. long, 2-4 row block.

Plots were harvested by machine, and yield is calculated by the on-board computer.

TABLE 15
Field testing of endophytes in Rhizoctonia treated corn, showing
% uplift (improvement) relative to untreated controls. Confirmed stress
data points represented confirmed Rhizoctonia stress plots.

| | | | Early | Full | Plant | Root | Shoot |
|---|---|---|---|---|---|---|---|
| Year | Trial | Treatment | Emergence | Emergence | Height | Weight | Weight |
| 1 | All | Chemical | −2.7 | −1 | 2.7 | 8.6 | 8.5 |
| | (12 data points) | treatment | | | | | |
| | All | MIC-70076 | 0.2 | −0.9 | 1.7 | 12.6 | 12.5 |
| | (12 data points) | | | | | | |
| 2 | All | Chemical | 11.5 | 7.7 | 7.1 | 10.2 | 10.3 |
| | (42 data points) | treatment | | | | | |
| | All | MIC-70076 | 4.5 | 1.5 | 2.3 | 1.5 | 5.7 |
| | (42 data points) | | | | | | |
| | Confirmed Stress | Chemical | 13.4 | 15.1 | 8.9 | 14.9 | 17.8 |
| | (24 data points) | treatment | | | | | |
| | Confirmed Stress | MIC-70076 | 0 | 2.6 | 0.3 | 4.3 | 5.7 |
| | (24 data points) | | | | | | |

### Example 21. Method of Preparation of Endophytes and Heterologous Disposition of Endophytes on Seeds for Field Trials

### Preparation of Endophytes

Bacteria: An agar plug of each bacterial strain is transferred using a transfer tube to 4 ml of potato dextrose broth (PDB) in a 24 well plate and incubated at room temperature at 675 rpm on a shaker for 3 days. After growth of bacteria in broth, 200 μl is transferred into a spectrophotometer reading plate and bacteria OD is read at 600 nm absorbance. All bacteria strains are then normalized to 0.05 OD utilizing PBS 1x buffer.

Fungi: Preparation of molasses broth and potato dextrose agar: Molasses broth is prepared by dissolving 30 g molasses and 5 g yeast extract per liter deionized water in an autoclavable container and autoclaving (15 psi, 121° C.) for 45 min. Potato dextrose agar (PDA) plates are prepared by dissolving 39.0 g PDA powder per liter deionized water in an autoclavable container and autoclaving (15 psi, 121° C.) for 45 min. The agar is allowed to cool to 50-60° C., before pouring into sterile petri plates (30 ml per 90 mm plate). Fungal endophyte treatments may be applied as either a dry or liquid formulation.

Liquid biomass: All equipment and consumables are thoroughly sterilized and procedures performed in a biosafety cabinet. The inoculant is prepared by placing 1 plug from a cryopreserved stock on a fresh PDA plate, sealing the plate with Parafilm® and incubating at room temperature in the dark for 5-10 days. Then ˜5×5 mm plugs are cut from the PDA plates and 10-12 plugs are transferred into flasks containing the sterile molasses broth, covered, secured in a shaker and incubated for at least 10 days with shaking at ˜130 rpm. Then the culture is placed in a blender for 5 seconds and 1 ml of the blended culture is centrifuged and the supernatant is discarded. The pellet is resuspended in 0.5 ml 1x Phosphate Buffered Saline (PBS) to generate inoculum.

Dry biomass: All equipment and consumables are thoroughly sterilized and procedures performed in a biosafety cabinet. The inoculant is prepared by placing 1 plug from a cryopreserved stock on a fresh PDA plate, sealing the plate with Parafilm® and incubating at room temperature in the dark for 5-10 days. Then ˜5×5 mm plugs are cut from the PDA plates and 10-12 plugs are transferred into flasks containing the sterile molasses broth, covered, secured in a shaker and incubated for at least 10 days with shaking at ˜130 rpm. In sterile conditions, the liquid culture is carefully decanted using 150 mm sterile filter paper on a sterilized Buchner funnel over a sterile flask. Once all liquid passes through the funnel, the pellet is rinsed with sterile water until the filtrate runs clear. When dry, the pellet is transferred to a drying cabinet and dried until brittle. The pellet is then ground into a fine powder, and sample is used to generate CFU counts.

### Preparation of Formulation for Seed Treatments

A 2% weight/volume solution of sodium alginate for the seed coatings is prepared by the following method. An Erlenmeyer flask is filled with the appropriate volume of deionized water and warmed to 50 degrees Celsius on a heat plate with agitation using a stir bar. The appropriate mass of sodium alginate powder for the desired final concentration solution is slowly added until dissolved. The solution is autoclaved at 121 degrees Celsius at 15 PSI for 30 minutes to sterilize.

Talc for the powdered seed coatings is prepared by the following method. Talc is aliquoted into bags or 50 ml Falcon tubes and autoclaved in dry cycle (121 degrees Celsius at 15 PSI for 30 minutes) to sterilize.

### Heterologous Disposition of Endophytes on Seeds

Seeds treated were heterologously disposed to each endophyte according to the following seed treatment protocol.

Liquid formulation: Liquid culture is added to the seeds at a rate of 23 (for fungal endophyte treatments) or 8.4 (for bacterial endophyte treatments) ml per kg of seeds, with equivalent volumes of the prepared sodium alginate. Control treatments are prepared using equivalent volumes of sterile broth. The seeds are then agitated to disperse the solution evenly on the seeds. For fungal endophytes, 15 g per kg of seed of talc powder as prepared above is added and the seeds are agitated to disperse the powder evenly on the seeds. Then 16.6 ml (for fungal endophyte treatments) or 2.4 ml (for bacterial endophyte treatments) per kg of seed of Flo-Rite® 1706 (BASF, Ludwigshafen, Germany) is added and the seeds are agitated to disperse the powder evenly on the seeds. Slightly less Flo-Rite® is used for small grains and canola seeds, slightly more Flo-rite® is used for seeds such as corn, soy, cotton and peanut seeds. The target dose is generally between 10\hat{ }0-10\hat{ }6 CFU per seed, in some cases at least 10\hat{ }3 CFU per seed, or at least 10\hat{ }4 CFU per seed. Treated seeds are allowed to dry overnight in a well-ventilated space before planting.

Dry formulation: The 2% sodium alginate solution prepared above is added to the seeds at a rate of 23 ml per kg of seeds. Equal parts of dry biomass and talc prepared as above are mixed. The solution is applied so that an equivalent of 10 g of powdered dry biomass is applied per kg of seeds. Control treatments are prepared using equivalent volumes of talc. The seeds are then agitated to disperse the solution evenly on the seeds. Then 16.6 ml per kg of seed of Flo-Rite® 1706 (BASF, Ludwigshafen, Germany) is added and the seeds are agitated to disperse the powder evenly on the seeds. Slightly less Flo-Rite® is used for small grains and canola seeds, slightly more Flo-rite® is used for seeds such as corn soy, cotton and peanut seeds. The target dose is generally between 10\hat{ }0-10\hat{ }6 CFU per seed, in some cases at least 10\hat{ }3 CFU per seed, or at least 10\hat{ }4 CFU per seed. Treated seeds are allowed to dry overnight in a well-ventilated space before planting.

### Example 22. Field Assessment of Improved Plant Characteristics

### Rice

Field trials are conducted, preferably, at multiple locations. In some embodiments, rice seeds are treated with commercial fungicidal and insecticidal treatment. Seeds are heterologously disposed with the endophyte treatments and formulation control (lacking the one or more heterologously disposed endophytes) as described in Example 21, untreated seeds (lacking formulation and the one or more heterologously disposed endophyte) are also planted. Seeds are sown in regularly spaced rows in soil at 1.2 million seeds/acre seeding density. At each location at least 3 replicate plots are planted for each endophyte or control treatments in a randomized complete block design. For example, each plot may consist of seven, 15.24 m (40 ft.) rows.

At the end of the field trial employing endophyte treatment and control treatment plants, plots are harvested, for example, by machine with a 5-ft research combine and yield is calculated by the on-board computer.

### Wheat

Field trials are conducted at multiple locations with multiple plots per location. Wheat seeds (optionally treated with commercial fungicidal and insecticidal treatments) are heterologously disposed with the endophyte treatments as described in Example 21; untreated seeds (lacking formulation and the one or more heterologously disposed endophyte) are also planted. Seeds are sown in regularly spaced rows in soil at 1.2 million seeds/acre seeding density. At each location at least 3 replicate plots are planted for each endophyte or control treatments in a randomized complete block design. Each plot consists of seven, 15.24 m (40 ft.) rows.

Plots are harvested by machine, for example with a 5-ft research combine and yield was calculated by the on-board computer.

### Corn

Field trials are conducted at multiple locations, preferably with multiple plots per location. Plots may be irrigated, non-irrigated (dryland), or maintained with suboptimal irrigation at a rate to target approximately 25% reduction in yield. In some embodiments, corn seeds are treated with commercial fungicidal and insecticidal treatment. Seeds are heterologously disposed with the endophyte treatments as described in Example 21; untreated seeds (lacking formulation and the one or more heterologously disposed endophyte) are also planted. Seeds are sown in regularly spaced rows in soil at planting densities typical for each region. At each location at least 3 replicate plots are planted per endophyte or control treatment in a randomized complete block design. For example, each plot may consist of four 15.24 m (40 ft.) rows, each separated by 76.2 cm (30 in).

At the end of the field trial employing endophyte treatment and control treatment plants, plots are harvested, for example, by machine with a 5-ft research combine and yield is calculated by the on-board computer. Only the middle two rows of the 4 row plots are harvested to prevent border effects.

### Soy

Field trials are conducted, preferably, at multiple locations. Seeds are heterologously disposed with the endophyte treatments as described in Example 21, untreated seeds (lacking formulation and the one or more heterologously disposed endophyte) are also planted. In some embodiments, soybean seeds are treated with commercial fungicidal and insecticidal treatment. Seeds are sown in regularly spaced rows in soil at planting densities typical for each region, for example, at 180,000 seeds/acre seeding density. At each location at least 3 replicate plots are planted per endophyte or control treatment in a randomized complete block design). For example, each plot may consist of four 15.24 m (40 ft.) rows, each separated by 76.2 cm (30 in).

At the end of the field trial employing endophyte treatment and control treatment plants, plots are harvested, for example, by machine with a 5-ft research combine and yield is calculated by the on-board computer. Only the middle two rows of the 4 row plots are harvested to prevent border effects.

### Canola

Field trials are conducted at multiple locations, preferably in diverse geographic regions. Plots may be irrigated, non-irrigated (dryland) or maintained with suboptimal irrigation at a rate to target approximately 25% reduction in yield. In some embodiments, canola seeds are treated with commercial fungicidal and insecticidal treatment. Seeds are heterologously disposed with the endophyte treatments as described in Example 21, untreated seeds (lacking formulation and the one or more heterologously disposed endophyte) are also planted. At each location, at least 3 replicate plots are planted for each endophyte or control treatment in a randomized complete block design.

At the end of the field trial employing endophyte treatment and control treatment plants, plots are harvested, for example, by machine with a 5-ft research combine and yield is calculated by the on-board computer.

### Peanut

Field trials are conducted at multiple locations, preferably in diverse geographic regions. Optionally, plots are non-irrigated (dryland) or maintained with suboptimal irrigation at a rate to target approximately 25% reduction in yield. In some embodiments, peanut seeds are treated with commercial fungicidal and insecticidal treatment. Seeds are heterologously disposed with the endophyte treatments as described in Example 21, untreated seeds (lacking formulation and the one or more heterologously disposed endophyte) are also planted.

At the end of the field trial employing endophyte treatment and control treatment plants, plots are harvested, for example, by machine with a 5-ft research combine and yield is calculated by the on-board computer.

### Example 23. Method of Determining Seed Nutritional Quality Trait Component: Fat

Seed samples from harvested plants are obtained as described in Example 22. Analysis of fat is conducted on replicate samples according to the Association of Official Agricultural Chemists Reference Method AOAC 920.39, of the Official Methods of Analysis of AOAC International, 20th Edition (2016), herein incorporated by reference in its entirety. Samples are weighed onto filter paper, dried, and extracted in hot hexane for 4 hrs. using a Soxlhet system. Oil is recovered in pre-weighed glassware, and % fat is measured gravimetrically. Mean percent change between the treatment (endophyte-treated seed) and control (seed treated) with the formulation calculated.

### Example 24. Method of Determining Seed Nutritional Quality Trait Component: Ash

Seed samples from harvested plants are obtained as described in Example 22. Analysis of ash is conducted on replicate samples according to the Association of Official Agricultural Chemists Reference Method AOAC 920.39, of the Official Methods of Analysis of AOAC International, 20th Edition (2016). Samples are weighed into pre-weighed crucibles, and ashed in a furnace at 600° C. for 3 hr. Weight loss on ashing is calculated as % ash. Mean percent change between the treatment (one or more heterologously disposed endophytes) and control (lacking the one or more heterologously disposed endophytes) with the formulation calculated.

### Example 25. Method of Determining Seed Nutritional Quality Trait Component: Fiber

Seed samples from harvested plants are obtained as described in Example 22. Analysis of fiber is conducted on replicate samples according to the Association of Official Agricultural Chemists Reference Method AOAC 920.39, of the Official Methods of Analysis of AOAC International, 20th Edition (2016). Samples are weighed into filter paper, defatted and dried, and hydrolyzed first in acid, then in alkali solution. The recovered portion is dried, weighed, ashed at 600° C., and weighed again. The loss on ashing is calculated as % Fiber. Mean percent change between the treatment (one or more heterologously disposed endophytes) and control (lacking the one or more heterologously disposed endophytes) with the formulation calculated.

### Example 26. Method of Determining Seed Nutritional Quality Trait Component: Moisture

Seed samples from harvested plants are obtained as described in Example 22. Analysis of moisture is conducted on replicate samples according to the Association of Official Agricultural Chemists Reference Method AOAC 920.39, of the Official Methods of Analysis of AOAC International, 20th Edition (2016). Samples are weighed into pre-weighed aluminum dishes, and dried at 135° C. for 2 hrs. Weight loss on drying is calculated as % Moisture. Mean percent change between the treatment (one or more heterologously disposed endophytes) and control (lacking the one or more heterologously disposed endophytes) with the formulation calculated.

### Example 27. Method of Determining Seed Nutritional Quality Trait Component: Protein

Seed samples from harvested plants are obtained as described in Example 22. Analysis of protein is conducted on replicate samples according to the Association of Official Agricultural Chemists Reference Method AOAC 920.39, of the Official Methods of Analysis of AOAC International, 20th Edition (2016). Samples are combusted and nitrogen gas is measured using a combustion nitrogen analyzer (Dumas). Nitrogen is multiplied by 6.25 to calculate % protein. Mean percent change between the treatment (one or more heterologously disposed endophytes) and control (lacking the one or more heterologously disposed endophytes) with the formulation calculated.

### Example 28. Method of Determining Seed Nutritional Quality Trait Component: Carbohydrate

Seed samples from harvested plants are obtained as described in Example 22. Analysis of carbohydrate is determined for replicate samples as a calculation according to the following formula: Total Carbohydrate=100%-% (Protein+Ash+Fat+Moisture+Fiber), where % Protein is determined according to the method of Example 27, % Ash is determined according to the method of Example 24, % Fat is determined according to the method of Example 21, % Moisture is determined according to the method of Example 26, and % Fiber is determined according to the method of Example 25. Mean percent change between the treatment (one or more heterologously disposed endophytes) and control (lacking the one or more heterologously disposed endophytes) are calculated.

### Example 29. Method of Determining Seed Nutritional Quality Trait Component: Calories

Seed samples from harvested plants are obtained as described in Example 22. Analysis of Calories is determined for replicate samples as a calculation according to the following formula: Total Calories=(Calories from protein)+(Calories from carbohydrate)+Calories from fat), where Calories from protein are calculated as 4 Calories per gram of protein (as determined according to the method of Example 27), Calories from carbohydrate are calculated as 4 Calories per gram of carbohydrate (as determined according to the method of Example 28), and Calories from fat are calculated as 9 Calories per gram of fat (as determined according to the method of Example 23). Mean percent change between the treatment (one or more heterologously disposed endophytes) and control (lacking the one or more heterologously disposed endophytes) are calculated.

### Example 30. Additional Methods for Creating Synthetic Compositions

### Osmopriming and Hydropriming

One or more endophytes are inoculated onto seeds during the osmopriming (soaking in polyethylene glycol solution to create a range of osmotic potentials) and/or hydropriming (soaking in de-chlorinated water) process. Osmoprimed seeds are soaked in a polyethylene glycol solution containing one or more endophytes for one to eight days and then air dried for one to two days. Hydroprimed seeds are soaked in water for one to eight days containing one or more endophytes and maintained under constant aeration to maintain a suitable dissolved oxygen content of the suspension until removal and air drying for one to two days. Talc and or flowability polymer are added during the drying process.

### Foliar Application

One or more endophytes are inoculated onto aboveground plant tissue (leaves and stems) as a liquid suspension in dechlorinated water containing adjuvants, sticker-spreaders and UV protectants. The suspension is sprayed onto crops with a boom or other appropriate sprayer.

### Soil Inoculation

One or more endophytes are inoculated onto soils in the form of a liquid suspension, either; pre-planting as a soil drench, during planting as an in-furrow application, or during crop growth as a side-dress. One or more endophytes are mixed directly into a fertigation system via drip tape, center pivot or other appropriate irrigation system.

### Hydroponic and Aeroponic Inoculation

One or more endophytes are inoculated into a hydroponic or aeroponic system either as a powder or liquid suspension applied directly to the rockwool substrate or applied to the circulating or sprayed nutrient solution.

### Vector-Mediated Inoculation

One or more endophytes are introduced in power form in a mixture containing talc or other bulking agent to the entrance of a beehive (in the case of bee-mediation) or near the nest of another pollinator (in the case of other insects or birds. The pollinators pick up the powder when exiting the hive and deposit the inoculum directly to the crop's flowers during the pollination process.

### Root Wash

The method includes contacting the exterior surface of a plant's roots with a liquid inoculant formulation containing one or more endophytes. The plant's roots are briefly passed through standing liquid microbial formulation or liquid formulation is liberally sprayed over the roots, resulting in both physical removal of soil and microbial debris from the plant roots, as well as inoculation with microbes in the formulation.

### Seedling Soak

The method includes contacting the exterior surfaces of a seedling with a liquid inoculant formulation containing one or more endophytes. The entire seedling is immersed in standing liquid microbial formulation for at least 30 seconds, resulting in both physical removal of soil and microbial debris from the plant roots, as well as inoculation of all plant surfaces with microbes in the formulation. Alternatively, the seedling can be germinated from seed in or transplanted into media soaked with the microbe(s) of interest and then allowed to grow in the media, resulting in soaking of the plantlet in microbial formulation for much greater time, for example: hours, days or weeks. Endophytic microbes likely need time to colonize and enter the plant, as they explore the plant surface for cracks or wounds to enter, so the longer the soak, the more likely the microbes will successfully be installed in the plant.

### Wound Inoculation

The method includes contacting the wounded surface of a plant with a liquid or solid inoculant formulation containing one or more endophytes. Plant surfaces are designed to block entry of microbes into the endosphere, since pathogens attempt to infect plants in this way. One way to introduce beneficial endophytic microbes into plant endospheres is to provide a passage to the plant interior by wounding. This wound can take a number of forms, including pruned roots, pruned branches, puncture wounds in the stem breaching the bark and cortex, puncture wounds in the tap root, puncture wounds in leaves, puncture wounds seed allowing entry past the seed coat. Wounds can be made using tools for physical penetration of plant tissue such as needles. Microwounds may also be introduced by sonication. Into the wound can then be contacted the microbial inoculant as liquid, as powder, inside gelatin capsules, in a pressurized capsule injection system, or in a pressurized reservoir and tubing injection system, allowing entry and colonization by microbes into the endosphere. Alternatively, the entire wounded plant can be soaked or washed in the microbial inoculant for at least 30 seconds, giving more microbes a chance to enter the wound, as well as inoculating other plant surfaces with microbes in the formulation—for example pruning seedling roots and soaking them in inoculant before transplanting is a very effective way to introduce endophytes into the plant.

### Injection

The method includes injecting microbes into a plant in order to successfully install them in the endosphere. Plant surfaces are designed to block entry of microbes into the endosphere, since pathogens attempt to infect plants in this way. In order to introduce beneficial endophytic microbes to endospheres, we need a way to access the interior of the plant which we can do by puncturing the plant surface with a needle and injecting microbes into the inside of the plant. Different parts of the plant can be inoculated this way including the main stem or trunk, branches, tap roots, seminal roots, buttress roots, and even leaves. The injection can be made with a hypodermic needle, a drilled hole injector, or a specialized injection system, and through the puncture wound can then be contacted the microbial inoculant as liquid, as powder, inside gelatin capsules, in a pressurized capsule injection system, or in a pressurized reservoir and tubing injection system, allowing entry and colonization by microbes into the endosphere.

### Example 31. Identification of Sequence Variants Across Core Genes

Phylogenomic analysis of whole genome sequences of endophytes can be used to identify distinguishing sequence variants. Sets of genes suitable for phylogenomic analysis as well as methods for identifying the same are well known in the art, for example Floutas et al. (2012) The Paleozoic origin of enzymatic lignin decomposition reconstructed from 31 fungal genomes. Science, 336 (6089): 1715-9. doi: 10.1126/science.1221748 and James T Y, Pelin A, Bonen L, Ahrendt S, Sain D, Corradi N, Stajich J E. Shared signatures of parasitism and phylogenomics unite Cryptomycota and microsporidia. Curr Biol. 2013; 23 (16): 1548-53. doi: 10.1016/j.cub.2013.06.057. Orthologous genes to the reference set are identified in protein data bases derived from the genome of each species. Orthologous genes can be identified in the genomes using methods well known including reciprocal best hits (Ward N, Moreno-Hagelsieb G. Quickly Finding Orthologs as Reciprocal Best Hits with BLAT, LAST, and UBLAST: How Much Do We Miss? de Crécy-Lagard V, ed. PLOS ONE. 2014; 9 (7): e101850. doi: 10.1371/journal.pone.0101850) and Hidden Markov Models (HMMs). The best hits are extracted and a multiple sequence alignment generated for each set of orthologous genes. The alignments are used to build phylogenetic trees using methods well known in the art including Bayesian inference and maximum likelihood methods, for example using software tools MrBayes (Huelsenbeck, J. P. & Ronquist (2001) MRBAYES: Bayesian inference of phylogenetic trees. Bioinformatics, 17 (8): 754-755) and RAxML (Stamatakis, A. (2014) RAXML version 8: a tool for phylogenetic analysis and post-analysis of large phylogenies. Bioinformatics, 30 (9): 1312-1313. doi: 10.1093/bioinformatics/btu033). Sequence variants which distinguish between closely related species are identified.

### Example 32. Identification of Unique Genes in an Endophyte of Interest

Whole genome analysis of endophytes can be used to identify genes whose presence, absence or over or under representation (“differential abundance”) are associated with desirable phenotypes. To identify genes with differential abundance in the genome of an endophyte of interest, protein sequences predicted from the genomes of the endophyte and closely related species are compared in an all-vs-all pairwise comparison (for example, using BLAST) followed by clustering of the protein sequences based on alignment scores (for example, using MCL: Enright A. J., Van Dongen S., Ouzounis C. A. An efficient algorithm for large-scale detection of protein families. Nucleic Acids Research 30 (7): 1575-1584 (2002)). Additional software tools useful for this analysis are well known in the art and include OMA, OrthoMCL and TribeMCL (Roth A C, Gonnet G H, Dessimoz C. Algorithm of OMA for large-scale orthology inference. BMC Bioinformatics. 2008; 9:518. doi: 10.1186/1471-2105-9-518, Enright A J, Kunin V, Ouzounis C A. Protein families and TRIBES in genome sequence space. Nucleic Acids Res. 2003; 31 (15): 4632-8; Chen F, Mackey A J, Vermunt J K, Roos D S. Assessing performance of orthology detection strategies applied to eukaryotic genomes. PLOS One. 2007; 2 (4): e383). The protein clusters are queried to identify clusters with differential abundance of proteins derived from endophytes having desirable phenotypes. Proteins of these clusters define the unique properties of these endophytes, and the abundance of genes encoding these proteins may be used to identify endophytes of the present invention.

Having illustrated and described the principles of the present invention, it should be apparent to persons skilled in the art that the invention can be modified in arrangement and detail without departing from such principles. It is to be understood that while the invention has been described in conjunction with the detailed description thereof, the foregoing description is intended to illustrate and not limit the scope of the invention, which is defined by the scope of the appended claims. Other embodiments, advantages, and modifications are within the scope of the present invention.

1. A synthetic composition, comprising one or more endophytes heterologously disposed to a treatment formulation, wherein the endophytes comprise at least one tailocin gene cluster, and wherein the endophyte in the synthetic composition is capable of improving a trait of agronomic importance in a plant or plant element heterologously disposed to the synthetic composition.

2. The synthetic composition of claim 1, wherein:
(a) the at least one gene of the tailocin gene cluster comprises at a polynucleotide sequence that is at least 97% identical to SEQ ID NO. 287-933; and/or
(b) the one or more endophytes comprise a plasmid containing one or more genes of the tailocin gene cluster;
(c) are capable of producing one or more proteins having amino acid sequences are at least 97% identical to SEQ ID NOs. 1318-1922;
(d) are capable of producing one or more proteins having amino acid sequences are at least 99% identical to SEQ ID NOs. 1318-1922;
(e) are capable of producing one or more proteins whose amino acid sequence is an amino acid sequence selected from SEQ ID NOs. 1318-1922;
(f) comprise a plasmid comprising an open reading frame encoding a protein whose amino acid sequence is at least 97% identical to an amino acid sequence selected from SEQ IDs. 1318-1922;
(g) comprise a plasmid comprising an open reading frame encoding a protein whose amino acid sequence is at least 99% identical to an amino acid sequence selected from SEQ IDs. 1318-1922; or
(h) comprise a plasmid comprising an open reading frame encoding a protein whose amino acid sequence is an amino acid sequence selected from SEQ IDs. 1318-1922.

3. (canceled)

4. The synthetic composition of claim 2, wherein the one or more genes of the tailocin gene cluster comprise one more polynucleotide sequences that are at least 97% identical to SEQ ID NO. 287-933.

5.-10. (canceled)

11. A synthetic composition, comprising one or more endophytes heterologously disposed to a treatment formulation, wherein the endophytes comprise at least one polynucleotide sequence of having at least 97% identity to one or more of (i) SEQ IDs. 1-239, (ii) SEQ IDs. 240-253, (iii) SEQ IDs. 254-286, (iv) SEQ IDs. 934-1014, or (v) SEQ IDs. 1015-1089, and wherein the endophyte in the synthetic composition is capable of improving a trait of agronomic importance in a plant or plant element heterologously disposed to the synthetic composition.

12. The synthetic composition of claim 11, wherein the one or more endophytes:
(a) comprise a plasmid containing one or genes having at least 97% identity to one or more of SEQ IDs. 1-239;
(b) are capable of producing one or more proteins having amino acid sequences are at least 97% identical to SEQ ID NOs. 1090-1272;
(c) comprise a plasmid comprising an open reading frame encoding a protein whose amino acid sequence is at least 97% identical to an amino acid sequence selected from SEQ IDs. 1090-1272;
(d) comprise a plasmid containing one or genes having at least 97% identity to one or more of SEQ IDs. 240-253;
(e) are capable of producing one or more proteins having amino acid sequences are at least 97% identical to SEQ ID NOs. 1273-1285;
(f) comprise a plasmid comprising an open reading frame encoding a protein whose amino acid sequence is at least 97% identical to an amino acid sequence selected from SEQ IDs. 1273-1285;
(g) comprise a plasmid containing one or genes having at least 97% identity to one or more of SEQ IDs. 254-286;
(h) are capable of producing one or more proteins having amino acid sequences are at least 97% identical to SEQ ID NOs. 1286-1317;
(i) comprise a plasmid comprising an open reading frame encoding a protein whose amino acid sequence is at least 97% identical to an amino acid sequence selected from SEQ IDs. 1286-1317;
(i) comprise a plasmid containing one or genes having at least 97% identity to one or more of SEQ IDs. 934-1014;
(k) are capable of producing one or more proteins having amino acid sequences are at least 97% identical to SEQ ID NOs. 1923-1981;
(l) comprise a plasmid comprising an open reading frame encoding a protein whose amino acid sequence is at least 97% identical to an amino acid sequence selected from SEQ IDs. 1923-1981;
(m) comprise a plasmid containing one or genes having at least 97% identity to one or more of SEQ IDs. 1015-1089;
(n) are capable of producing one or more proteins having amino acid sequences are at least 97% identical to SEQ ID NOs. 1982-2051; or
(o) comprise a plasmid comprising an open reading frame encoding a protein whose amino acid sequence is at least 97% identical to an amino acid sequence selected from SEQ IDs. 1982-2051.

13.-50. (canceled)

51. A synthetic composition, comprising one or more endophytes heterologously disposed to a treatment formulation, wherein the endophytes comprise one or more of:
(a) a flagellin gene having at least 97% identity to one or more of (i) SEQ IDs. 1-8 or 14-16, (ii) SEQ IDs. 9, 55, 56, 130-133, (iii) SEQ IDs. 9, 11-13, 17-18, 20-23, or (iv) SEQ IDs. 10 or 19,
(b) an O-antigen biosynthesis gene having at least 97% identity to one or more of SEQ IDs. 240-243,
(c) a pseudaminic acid biosynthesis gene having at least 97% identity to one or more of (i) SEQ IDs. 256-262 or (ii) SEQ IDs. 254-255,
(d) a gene of a tailocin gene cluster having at least 97% identity to one or more of (i) SEQ IDs. 288-342, (ii) SEQ IDs. 288-289 or 566-605, (iii) SEQ IDs. 287, 343, 382-390, or (iv) SEQ IDs. 375-381,
(e) a gene of a Type IV secretion system having at least 97% identity to one or more of (i) SEQ IDs. 938-941, (ii) SEQ IDs. 974-977, (iii) SEQ IDs. 934-937, or (iv) SEQ ID. 944, and
(f) a gene of a Type IV secretion system putative effector having at least 97% identity to one or more of (i) SEQ IDs. 1018-1022, (ii) SEQ IDs. 1054-1057, or (iii) SEQ IDs. 1015-1017 or 1024, and
wherein the endophyte in the synthetic composition is capable of improving a trait of agronomic importance in a plant or plant element heterologously disposed to the synthetic composition.

52.-54. (canceled)

55. A synthetic composition, comprising one or more endophytes heterologously disposed to a treatment formulation, wherein:
(a) the endophytes comprise one or more polynucleotide sequences at least 97% identical to one or more of SEQ ID NOs. 1-8, and the one or more endophytes are capable of producing one or more proteins whose amino acid sequences are selected from SEQ ID NOs. 1090-1096, wherein the endophyte in the synthetic composition is capable of improving biotic stress tolerance in an environment containing pathogenic Fusarium in a plant or plant element heterologously disposed to the synthetic composition;
(b) the endophytes comprise one or more polynucleotide sequences at least 97% identical to one or more of SEQ ID NOs. 9-13, 254, 255, 287, 934-937, 1015-1017, 1-8, 24-28, 75-85, 365-374, 442-452, 959-962, 1023, 1038-1041, and the one or more endophytes are capable of producing one or more proteins whose amino acid sequences are selected from SEQ ID NOs. 1097-1103, 1286, 1287, 1318, 1319, 1923-1926, 1982-1984, 1090-1096, 1113, 1114, 1149-1157, 1402-1409, 1471-1483, 1946-1947, 1990, 2005, 2006, wherein the endophyte in the synthetic composition is capable of improving biotic stress tolerance in an environment containing pathogenic Rhizocotina in a plant or plant element heterologously disposed to the synthetic composition; or
(c) the endophytes comprise one or more polynucleotide sequences at least 97% identical to one or more of SEQ ID NOs. 1-18, 240-243, 254-262, 287-364, 934-943, 1015-1022, 19-74, 365-374, 391-441, 945-958, 1023, 1025-1037, and the one or more endophytes are capable of producing one or more proteins whose amino acid sequences are selected from SEQ ID NOs. 1090-1106, 1112, 1273-1276, 1286-1294, 1318-1401, 1923-1934, 1982-1989, 1107-1111, 1113-1148, 1402-1409, 1426-1470, 1936-1945, 1990, 1992-2004, wherein the endophyte in the synthetic composition is capable of improving biotic stress tolerance in an environment containing pathogenic Pythium in a plant or plant element heterologously disposed to the synthetic composition.

56.-57. (canceled)

58. The synthetic composition of claim 1, wherein the synthetic composition additionally comprises one or more plant elements.

59. The synthetic composition of claim 58, wherein the one or more plant elements are:
(a) seeds;
(b) monocots; or
(c) dicots.

60. (canceled)

61. The synthetic composition of claim 59, wherein:
(a) the dicots are selected from the group consisting of cotton, tomato, lettuce, peppers, cucumber, endive, melon, potato, cannabis, and squash;
(b) the dicots are legumes; or
(c) the monocot is a cereal.

62. (canceled)

63. The synthetic composition of claim 61, wherein:
(a) the legume is soybean; or
(b) the cereal is a wheat and wherein the wheat is winter wheat.

64.-65. (canceled)

66. The synthetic composition of claim 1, wherein the synthetic composition is formulated such that the endophyte can be applied to seeds a rate of 0.65 mL/kg seed.

67. The synthetic composition of claim 59, wherein the synthetic composition comprises at least 1E+03 endophytes per seed, at least 1E+04 CFU per seed, at least 1E+05 CFU per seed, at least 1E+06 CFU per seed, at least 1E+07 CFU per seed, or at least 1E+08 CFU per seed.

68. The synthetic composition of claim 1, wherein the one or more endophytes are capable of improving one or more traits of agronomic importance in the plant element or plant derived from the plant element relative to a reference plant or plant derived from a reference plant element.

69. The synthetic composition of claim 68, wherein the one or more traits of agronomic importance comprise one or more of biotic stress tolerance, shoot fresh weight, yield, plant height, shoot weight, and or root weight.

70. The synthetic composition of claim 69, wherein the biotic stress is a growth environment comprising one or more pests or pathogens.

71. The synthetic composition of claim 70, wherein the one or more pests or pathogens is a Dreschlera, Bipolaris, Pythium, Rhizoctonia, or Fusarium species.

72. The synthetic composition of claim 1, wherein:
(a) the one or more endophytes are of the genus Kosakonia; or
(b) the one or more endophytes are of the genus and species Kosakonia cowanni.

73. (canceled)

74. The synthetic composition of claim 1, wherein the treatment formulation comprises one or more of:
(a) liquid state fermentation broth;
(b) one or more solid carrier;
(c) one or more adherent;
(d) talc and mineral oil;
(e) kaolin clay, a dispersant, and a surfactant; or
(f) a sugar.

75.-79. (canceled)

80. A method comprising applying the synthetic composition of claim 1 to a plant element.
